(19) United States
(12) Patent Application Publication
(10) Pub. No.: US 2021/0388441 A1
(43) Pub. Date: Dec. 16, 2021

(54) PREDICTING CHRONIC ALLOGRAFT INJURY THROUGH ISCHEMIA-INDUCED DNA METHYLATION

(71) Applicants: VIB VZW, Gent (BE); KATHOLIEKE UNIVERSITEIT LEUVEN, K.U.LEUVEN R&D, Leuven (BE)

(72) Inventors: Diether LAMBRECHTS, Kessel-Lo (BE); Line HEYLEN, Leuven (BE)

(21) Appl. No.: 16/956,204

(22) PCT Filed: Dec. 21, 2018
(86) PCT No.: PCT/EP2018/086509

§ 371 Date: Jun. 19, 2020

(30) Foreign Application Priority Data
Dec. 22, 2017 (EP) 17210414.3

(51) Int. Cl. C12Q 1/6883; G16B 20/20

(52) U.S. Cl. CPC C12Q 1/6883; C12Q 2600/154; G16B 20/20

(57) ABSTRACT

The present invention relates to the identification of a specific set of CpG biomarkers for predicting the risk of developing chronic allograft injury in a patient, and means and methods for preservation of allografts and transplantation organs. In particular, a method to predict the risk of developing chronic allograft injury in a patient is presented based on cold-ischemia induced hypermethylation of CpGs as an important driver for downregulation of (promoters of) genes essential for organ preservation. Specifically, a CpG biomarker signature for hypermethylation of renal allograft organs caused by hypoxia and ischemia pre-implantation revealed treatment options of ischemia-associated chronic allograft injury and preservation of donor kidneys.

### CROSS-REFERENCE TO RELATED APPLICATIONS

This application is a national phase entry under 35 U.S.C. § 371 of International Patent Application PCT/EP2018/086509, filed Dec. 21, 2018, designating the United States of America and published in English as International Patent Publication WO 2019/122303 A1 on Jun. 27, 2019, which claims the benefit under Article 8 of the Patent Cooperation Treaty to European Patent Application Serial No. 17210414.3, filed Dec. 22, 2017, the entireties of which are hereby incorporated by reference.

### FIELD OF THE INVENTION

The present invention relates to the identification of a specific set of CpG biomarkers for predicting the risk of developing chronic allograft injury in a patient, and means and methods for preservation of allografts and transplantation organs. In particular, a method to predict the risk of developing chronic allograft injury in a patient is presented based on cold-ischemia induced hypermethylation of CpGs as an important driver for downregulation of (promoters of) genes essential for organ preservation. Specifically, a CpG biomarker signature for hypermethylation of renal allograft organs caused by hypoxia and ischemia pre-implantation revealed treatment options of ischemia-associated chronic allograft injury and preservation of donor kidneys.

### BACKGROUND

DNA methylation is the attachment of a methyl group to cytosines located in a CpG dinucleotide context, creating a 5-methylcytosine (5mC). CpG dinucleotides (CpGs) tend to cluster in so-called CpG islands, mostly within enhancers, the promoter or first exon of genes, and when they are methylated this correlates with transcriptional silencing of the affected gene. DNA methylation represents a relatively stable but reversible epigenetic mark⁶. Its removal can be initiated by ten-eleven translocation (TET) enzymes, which convert 5mC to 5-hydroxymethylcytosine (5hmC) in an oxygen-dependent manner⁷. Recently, it was demonstrated that hypoxia reduces TET activity, leading to the accumulation of 5mC and loss of 5hmC. In cancer cells, this caused hypermethylation at promoters of tumour suppressor genes⁸. Specifically, because cancer cells are highly proliferative and subject to strong genetic selection, these hypermethylation events are strongly selected for and progressively accumulate in cancer cells. Other medical conditions are, however, also characterized by long-lasting oxygen shortage, but in these affected tissues are far less proliferative, raising the question whether also here DNA de-methylation activity is impaired and whether this similarly results in hypermethylation driving disease progressions. For instance, DNA methylation changes affecting the Ras oncoprotein inhibitor RASAL1 have been proposed to underlie kidney fibrosis, which is a key pathological feature contributing to chronic allograft injury (CAI) following kidney transplantations. However, besides this one report focusing on methylation events in RASAL1, DNA methylation has been very poorly characterized in the context of kidney transplantation.

Kidney transplantation is the treatment of choice for patients with end-stage renal failure. Despite the development of potent immune suppressive therapies, which improve outcome early after transplantation, annually 3-5% of grafts show late graft failure, with devastating consequences for patient quality of life and survival. Chronic allograft injury represents a leading cause for this late graft loss, and has been linked to ischemia-reperfusion injury (IRI) occurring during transplantation. In kidney transplantation, cold ischemia time is directly proportional to delayed functioning of grafted kidneys¹, overall reduced allograft function², and chronic allograft injury³. Despite intensive research, the pathophysiological mechanisms underlying ischemia-induced CAI are still insufficiently characterized. Experimental studies have highlighted that cold ischemia can trigger a complex set of events that delay graft function and sustain renal injury. For instance, acute ischemia can lead to chronic activation of the host immune response to the allograft⁴. Immunological as well as non-immunological insults leading to interstitial fibrosis and tubular atrophy culminate in injury and kidney failure, which was shown to be correlated to DNA methylation changes²⁵. Epigenome-wide studies assessing methylation levels to determine response to a specific cancer treatment has pinpointed a panel of specific methylation markers (Spinella et al. WO2014/025582A1). Similarly, an epigenome-wide methylation analysis on the effects of ischemia on kidneys could potentially link renal ischemia-induced epigenetic changes to kidney allograft injury, but has never been addressed. Chronic allograft injury or nephropathy predictive biomarkers based on differential gene expression levels identified so far all involve complex methods including mRNA analysis and therefore highly depend on timing of sampling and accuracy (for instance see Scherer, US2010/0022627A1 and Murphy et al. US2017/0114407A1). Though, since ischemia during kidney transplantation is a major cause of CAI, and since kidneys have the unique advantage that they are amenable for repeated biopsying allowing pre- versus post-ischemic DNA methylation changes to be accurately assessed within a single kidney, it would be interesting to explore whether DNA hypermethylation underlies ischemia-induced chronic kidney allograft injury. In fact, there are currently no biomarkers to predict or effective treatment options to avoid ischemia-associated CAI. So there is a need to determine how ischemia-reperfusion induces late allograft survival failure, and how this adverse outcome or increased risk of developing CAI can be predicted to obtain insights to avoid implantation of damaged organs, and to develop novel treatments.

### SUMMARY OF THE INVENTION

The present invention is based on a genome-wide study of the DNA methylation profile measured in renal allograft biopsies in 3 different cohorts at different time points during the transplantation process, demonstrating that DNA hypermethylation changes underlie chronic allograft failure after kidney transplantation. As DNA methylation is generally considered to be reversible and DNA methylation inhibitors are already approved for the treatment of hematological tumours, the current results have important therapeutic applications for the prevention of chronic allograft injury (CAI), a disease for which currently no therapy exists. The present invention is based on the development of a validated CpG biomarker methylation risk score (MRS) that can be measured at implantation and that predicts the risk of developing CAI up to one year later, thereby revealing a novel epigenetic basis for ischemia-induced CAI with biomarker potential. Moreover, the predictive effect of said CpG biomarker MRS outperforms that of clinical variables currently routinely measured in the clinic. The present method has several advantages over the current measures such as the fact that DNA methylation is an attractive biomarker, as it is less sensitive to tissue handling compared to RNA and can even be performed on DNA isolated from small amounts of fixed tissue. So by detection of methylation levels, those methylation biomarkers improve the reliability, robustness, consistency and ease of handling as compared to other conventional biomarker methods, such as differential gene expression. Moreover, the methylation levels of CpGs measured at baseline, i.e. at the point of implantation, a strong correlation was found to future injury at 12 months, but not to injury already present at baseline. So, the use of these methylation markers not only has a predictive power superior to standard clinical variables currently used, but also has the advantage of monitoring a stable but reversible event, for which therapeutic agents are already established. In fact, the allograft or donor organ may be treated to reverse DNA methylation of those methylated markers disclosed herein prior to implantation, which so allows to preserve the donor organ, thereby also preventing systemic side effects. Alternatively, the lasting effect of ischemia on graft fibrosis observed in this disclosure suggests that inhibitors of DNA methylation form interesting therapeutic agents for improving outcome after transplantation or to prevent fibrosis and/or CAI. In addition to renal transplantation, other ischemic diseases, such as stroke and myocardial infarction allow to collect biopsies to correlate DNA methylation changes to the ischemia-induced damage in the tissue.

In a first aspect, the invention relates to a method for predicting the risk of developing chronic allograft injury in a patient that is eligible for receiving an allograft, comprising the steps of: a) determining the DNA methylation level of a CpG panel, comprising at least 4 CpGs from the list of CpGs shown in Table 4, in a sample of said allograft, donor organ or tissue; b) calculating a methylation risk score (MRS) via the sum of methylation values of each CpG in said CpG panel used in step a); c) comparing the MRS of the allograft sample with the MRS of a reference population, or with a population of reference organs; and d) attributing a higher risk of developing CAI when the MRS of the allograft sample is at least two-fold higher as compared to the MRS of the allograft samples of the lower tertile of the reference population. In said reference population, the MRS value is used to rank the allograft samples from low to high MRS, implying a ranking from low to high risk of developing CAI, and divide said population into 3 equal parts or tertiles for further comparison with newly developed MRS values of new samples of allografts.

Another embodiment relates to the CpG panel of at least 4 CpGs as determined in step a) in the method of the present invention, wherein said CpG panel comprises the 29 CpGs listed in Table 4. Another embodiment relates to the CpG panel of at least 4 CpGs as determined in step a) in the method of the present invention, wherein said CpG panel comprises the 413 CpGs listed in Table 3. In fact, those CpGs listed in Table 3 also contain said 29 CpGs of Table 4 (see upper part of Table 3). Another embodiment relates to the CpG panel of at least 4 CpGs as determined in step a) in the method of the present invention, wherein said CpG panel comprises the 1238 CpGs as listed in Table 6. Another embodiment relates to the CpG panel of at least 4 CpGs as determined in step a) in the method of the present invention, wherein said CpG panel comprises the 1634 CpGs listed in Table 2. In fact, those CpGs listed in Table 2 also contain said 29 CpGs of Table 4 (see Example 7).

In one embodiment, the allograft of said method for predicting the risk of developing CAI is a kidney. A particular embodiment discloses said method for predicting the risk of developing CAI, wherein the sample of the allograft is taken at the time of implantation. Alternative embodiments relate to a method wherein the sample of the allograft is taken before transplantation or after transplantation.

A particular embodiment relates to said method wherein the allograft sample is a biopsy sample from an allograft. Another embodiment relates to said method wherein the allograft sample is a liquid biopsy sample from said allograft.

Another aspect of the invention relates to an inhibitor of hypermethylation for use in preservation of the allograft prior to implantation or transplantation, wherein a higher risk of developing chronic allograft injury in a patient was predicted for said allograft according to the method of the present invention, relying on DNA methylation levels for a number of CpGs. Alternatively, for allografts wherein a higher risk of developing chronic allograft injury upon transplantation in a patient was predicted for said allograft using the method of the invention, a stimulator or enhancer of ten-eleven translocation (TET) enzyme activity is disclosed, for use in preservation of the allograft prior to implantation. Specifically, one embodiment relates to a stimulator of TET enzyme activity, for use in preservation of the allograft prior to implantation, wherein said stimulator is an inhibitor of the Branched-chain aminotransferase 1 (BCAT1) enzyme. In a preferred embodiment, said inhibitor of hypermethylation or stimulator of TET enzyme activity, is used for preservation of the allograft prior to implantation, when an allograft was predicted to have a higher risk of developing CAI in a patient, according to the method as described herein, involving the methylation of a specific CpG panel, comprising at least 4 CpGs from the list shown in Table 4. In the most preferred embodiment, said higher risk of developing CAI is hence determined or predicted using the method of the present invention, wherein the CpG panel used comprises at least 4 CpGs from Table 4, or comprises 29 CpGs from Table 4, or comprises 413 CpGs from Table 3, or comprises 1238 CpGs as listed in Table 6, or comprises 1634 CpGs from Table 2. Preferably said sample for said method is taken at the time of implantation, or prior to implantation. Alternatively, said sample is taken post-implantation, after which treatment of the patient for which a higher risk of developing CAI has been determined according to the method of the invention in said sample, is applied using an inhibitor of hypermethylation or a stimulator of TET activity, such as BCAT1, as a medicament.

Another aspect of the invention relates to the use of a panel of CpGs in a method for prediction of the risk of developing CAI, wherein said CpG panel comprises at least 4 CpGs of the CpGs listed in Table 4. In an alternative embodiment, said use of the biomarker CpG panel of at least 4 CpGs of the CpGs in Table 4 for prediction of the risk of developing CAI, comprises all 29 CpGs as listed in Table 4, or comprises the 413 CpGs as listed in Table 3, or comprises 1238 CpGs as listed in Table 6, or comprises the 1634 CpGs as listed in Table 2, wherein said CpGs listed in Table 2 and 3 contain the 29 CpGs also listed in Table 4 (see Examples). In a particular embodiment, said use of the biomarker CpG panel for prediction of the risk of developing CAI relates to an allograft being a kidney.

Another aspect of the invention relates to a kit for use in the method of the invention, or to the use of a kit for determining the DNA methylation level of a CpG panel, comprising detection means, such as oligonucleotides such as probes or primers, and optionally comprising further means, to measure the CpG methylation level of at least 4 CpGs from the list shown in Table 4. One embodiment relates to the use of said kit, for predicting the risk of developing CAI in a patient, more preferably, for predicting the risk of developing renal CAI in a patient. In one embodiment, the use of said kit is for determining the DNA methylation level of CpGs in the method for predicting the risk of developing CAI in a patient eligible for receiving an allograft.

### DESCRIPTION OF THE FIGURES

The drawings described are only schematic and are non-limiting. In the drawings, the size of some of the elements may be exaggerated and not drawn on scale for illustrative purposes.

FIG. 1. Schematic overview of the study cohorts to identify ischemia-induced DNA hypermethylation during kidney transplantation, and evaluate its functional implications.

FIG. 2. Genome-wide DNA methylation changes during kidney transplantation in paired pre-ischemic procurement and post-ischemic reperfusion biopsies.

Genome-wide DNA hypermethylation during kidney transplantation in post-ischemic reperfusion biopsies compared to the paired pre-ischemic procurement biopsies (n=2×13). (A) Median overall DNA methylation levels of kidney transplants before and after ischemia. The increase in methylation is significant for all transplants (P<0.0001, paired Mann-Whitney U test). (B) Logarithmic P values of changes in methylation at individual CpGs in paired kidney transplants comparing post versus pre-ischemia conditions. Peaks with a gain (right) or loss (left) in 5mC are highlighted at P<0.05. (C) Distribution of the T-statistics of paired tests on CpGs combined per island, for all islands, demonstrating the skewing towards hypermethylation of kidney transplants after ischemia. (D) Difference in DNA methylation after ischemia in and around the CpG island chr6:30852102-30852676 located in the promoter of DDR1, demonstrating diffuse hypermethylation of this region.

FIG. 3. Genome-wide loss of DNA hydroxymethylation upon ischemia.

Genome-wide loss of hydroxymethylation upon ischemia. (A) Overall DNA hydroxymethylation levels of transplants before (left bar) and after (right bar) ischemia. The decrease in hydroxymethylation is significant for all transplants (P<0.0001, paired t-test). Boxes are interquartile ranges, with mean as the white dot and median as the darker line. (B) 5hmC/C levels measured by LC-MS demonstrates a significant loss of 5hmC in kidney transplant biopsies from deceased donation (mean 17 h cold ischemia time; n=5) compared to living donation (<1 h; n=5). (C) Changes in 5mC levels against changes in 5hmC after ischemia. Colored points depict CpGs for which the change in 5hmC and 5mC are significant at P<0.05, with red used for the inverse relationship between 5mC and 5hmC and blue for the direct relationship.

FIG. 4. Genome-wide methylation changes during kidney transplantation in the cross-sectional cohort of post-ischemia pre-implantation biopsies.

Genome-wide methylation changes according to cold ischemia time during kidney transplantation in the cross-sectional cohort of post-ischemia pre-implantation biopsies (n=82). (A) Logarithmic P values obtained for individual CpGs that were correlated with the duration of cold ischemia time while adjusting for donor age and gender. Peaks with a gain (right) or loss (left) in 5mC are highlighted at P<0.05. (B) Distribution of the CpGs hypermethylated upon ischemia in both cohorts (right bars) versus all probes (left bars) according to their relationship with CpG islands. (C) Observed/expected fraction of ischemia-hypermethylated CpGs overlapping different kidney chromatin states. (D) Logarithmic P values obtained for CpG islands, which were correlated with the duration of cold ischemia time while adjusting for donor age and gender. Peaks gaining (right) and losing (left) are highlighted at FDR<0.05 and P<0.05 (light grey). (E) CpG islands hypermethylated in the pre-implantation cohort were also more likely to be hypermethylated in the longitudinal cohort.

FIG. 5. Functional annotation and expression changes of genes hypermethylated in transplanted kidneys.

(A) Pathway enrichment and (B) gene ontology enrichment of the genes associated with the 66 CpG islands that were hypermethylated after ischemia in both the longitudinal and pre-implantation cohorts. (C) Log fold change in the expression of hypermethylated genes after versus before ischemia in the longitudinal cohort (n=2×13). Each boxplot represents one transcript, in red when expression is reduced after ischemia (median log fold change below 1) and in blue when expression in increased after ischemia (median log fold change above 1). *P<0.05 by Wilcoxon test.

FIG. 6. Clinical relevance of ischemia-induced DNA hypermethylation in the 66 CpG islands that were consistently hypermethylated upon ischemia in both cohorts.

Clinical relevance of ischemia-induced DNA hypermethylation in the 66 CpG islands that were consistently hypermethylated upon ischemia in both cohorts. (A) Average DNA methylation changes of CpGs in the 66 CpG islands of kidney transplants post-ischemia post-reperfusion, at 3 months and 1 year after transplantation in the longitudinal cohort, compared to their pre-ischemia procurement samples, demonstrating the stability of the hypermethylation. (B) Relative risk of developing chronic allograft injury at 1 year after transplantation after stratifying patients into tertiles based on the methylation risk score. Odds ratios are shown for the pre-implantation cohort and replicated in the post-reperfusion cohort. (C and D) ROC curves for the methylation risk score (most left line) to predict chronic injury at 1 year after transplantation, compared to baseline clinical variables (donor age, donor last serum creatinine, expanded versus standard criteria donation, cold and warm ischemia time, and number of HLA mismatch (second line from the left). Curves are shown for the pre-implantation cohort (C) and replicated in the post-reperfusion cohort (D). (E and F) CADI score for each tertile based on the methylation risk score in the pre-implantation and post-reperfusion cohort. (G and H) Allograft function by tertile of methylation risk score in the pre-implantation and post-reperfusion cohort.

FIG. 7. Relative usage of each CpG in the 1000 minimal LASSO's.

### DETAILED DESCRIPTION TO THE INVENTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

The method and means provided by the invention allow to predict, prevent and provide treatment for chronic allograft injury (CAI) and/or fibrosis caused by cold ischennia-induced hypermethylation of allograft tissue, for instance donor organs such as kidneys. These findings are based on the first genome-wide profiling of the DNA methylation across >450.000 CpG sites using 3 different cohorts of human brain-dead donor kidney allograft biopsies: a longitudinal cohort with paired biopsies at procurement (n=13), after implantation and reperfusion (n=13), and at 3 or 12 months after transplantation (n=5 for both); a cross-sectional cohort with pre-implantation biopsies after cold ischemia (n=82); and a cross-sectional cohort with post-reperfusion biopsies (n=46). CAI was defined by an elevated Chronic Allograft Damage Index (CADI) score >2 at 3 and 12 months after transplantation. CADI is a pathology scoring system originally described by Isoniemi et al. 1992 (Kidney Intl 41:155-160). The composite CADI score is the sum of six individual scores represented by numbers (0 to 3) reflecting the extent or severity of the individual pathological features. Another scoring system is the Banff classification (Racusen et al. 1999, Kidney Int 55:713). How both systems relate to each other is discussed by Colvin 2007 (Transplantation 83:677-678).

In fact, the DNA methylation levels of kidney allografts that increased after ischemia in the longitudinal cohort were shown not to be transient, as DNA methylation was still increased up to 1 year after transplantation. The reversibility of DNA methylation however allowed to look for preservation of organs via a treatment that reverts these methylation events in the damaged tissues. Furthermore, the development and calculation of a Methylation Risk score (MRS) surprisingly outperforms baselines clinical variables in predicting outcome. More specifically, based on 66 CpG islands validated as the most consistently hypermethylated by ischemia in both cohorts (FDR<0.05), this MRS was capable to predict chronic allograft injury (CADI>2) at 1 year after transplantation (AUC 0.919) already in pre-implantation kidney biopsies. Of all 6 CADI score lesions, the score was highest for fibrosis and glomerulosclerosis. These findings provides a direct link between DNA hypermethylation events, arising due to ischemia during transplantation, and CAI, particularly fibrosis and glomerulosclerosis/fibrosis of glomeruli. Surprisingly, these hypermethylation events can be combined into an MRS that outperforms clinical variables in predicting CAI. Finally, those findings reveal novel treatment options to preserve allograft tissue and to prevent chronic injury, especially in kidney transplantation, via reverting hypermethylation or hypomethylation of those CpGs. Preclinical work has identified e.g. azacytidine and Jnk-inhibitors as having the potential to halt kidney fibrosis (Bechtel 2010, Nat Med 16:544; Yang 2010, Nat Med 16:535).

In a first aspect, the invention relates to a method for predicting the risk of developing CAI in a patient that is eligible for receiving the allograft, comprising the steps of: a) determining the DNA methylation level of a CpG panel, comprising at least 4 CpGs from the list of CpGs as shown in Table 4, in a sample of an allograft, b) calculating a MRS via the sum of methylation values of each CpG of said CpG panel, c) comparing the MRS of the sample of the allograft with a reference population of allografts, d) attributing a higher risk of developing chronic allograft injury when the MRS is at least two fold the MRS of the lowest tertile of the reference population.

As used herein the term “gene” refers to a genomic DNA sequence that comprises a coding sequence associated with the production of a polypeptide or polynucleotide product (e.g., rRNA, tRNA). The “methylation level” of a gene as used herein, encompasses the methylation level of sequences which are known or predicted to affect expression of the gene, including the promoter, enhancer, and transcription factor binding sites. As used herein, the term “enhancer” refers to a cis-acting region of DNA that is located up to 1 Mbp (upstream or downstream) of a gene. The term “CpG” as used herein is known in the art as dinucleotides of cytosine (C)-guanine (G) bases in the deoxyribonucleic acid chain. CpGs occur at certain locations or positions on the chromosomes at particular chromosomes, as indicated for each of the specific CpGs in Tables 2, 3, and 4, which were found to be hypermethylated in damaged allografts causal for graft fibrosis and CAI after transplantation in a patient or subject. CpGs are clustered on so-called CpG islands, for which the chromosomal start and end position defines their identity within the genome. The CpGs listed in Tables 2, 3 and 4 were also annotated to the gene regions wherein the CpGs or CpG islands are located in the genome, and their respective positions on the chromosomes refer to the ones in the Genome Reference Consortium Human Hg19 Build #37 assembly.

A “patient” or “subject”, for the purpose of this invention, relates to any organism such as a vertebrate, particularly any mammal, including both a human and another mammal, e.g., an animal such as a rodent, a rabbit, a cow, a sheep, a horse, a dog, a cat, a llama, a pig, or a non-human primate (e.g., a monkey). In one embodiment, the subject is a human, a rat or a non-human primate. Preferably, the subject is a human. In one embodiment, a subject is a subject with or suspected of having a disease or disorder, or an injury, also designated “patient” herein. In another embodiment, a subject is a subject ready to receive a transplant or allograft, also designated as a “patient eligible for receiving an allograft”.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, injury, or disease, but does not necessarily involve a total elimination of all disease-related signs, symptoms, conditions, or disorders. The term “preservation” in this invention relates to allograft or organ preservation, and means to maintain, keep, or ensure high quality, undamaged donor organs for delivery to a receiving subject, to allow the capability of rapid resumption of life-sustaining function in the recipient or patient. The process of organ transplantation is a medical procedure that involves the removal of an organ from a donor body, optionally storing or incubating this organ for transportation, and allowing it to be transplanted into another person's or recipient's body, to replace a damaged or missing organ, all while preserving the organ without significant damage. Several techniques are known by a skilled person for organ preservation such as static cold storage, normothermic machine perfusion, hypothermic machine perfusion, or combinations thereof. Organs that have been successfully transplanted include the heart, kidneys, liver, lungs, pancreas, intestine, and thymus. Some organs, like the brain, cannot be transplanted. Tissues for transplantation include bones, tendons (both referred to as musculoskeletal grafts), corneae, skin, heart valves, nerves and veins. Worldwide, the kidneys are the most commonly transplanted organs, followed by the liver and then the heart.

The term “allograft” is used herein to define a transplant of an organ or tissue from one individual to another of the same species with a different genotype. For example, a transplant from one person to another, but not an identical twin, is an allograft. Allografts account for many human transplants, including those from cadaveric, living related, and living unrelated donors. Also known as an allogeneic graft or a homograft. Allografts may consist of cells, tissue, or organs. “Allograft sample” or “sample of an allograft” may be obtained as a biopsy, more specifically a liquid biopsy, comprising blood or serum, or a solid biopsy, comprising cells or tissue.

As used herein, the term “sample methylation profile” or “DNA methylation” refers to the methylation levels at one or more target sequences in a sample's DNA, preferably an allograft sample's genomic DNA. The methylated DNA may be part of a sequence as an individual CpG locus or as a region of DNA comprising multiple CpG loci, for example, a gene promoter or CpG island. The methylation measured for the CpGs of the DNA of a sample tested according the methods disclosed herein is referred to as the DNA methylation level. As used herein, a “CpG island” refers to a G:C-rich region of genomic DNA containing an increased number of CpG dinucleotides relative to total genomic DNA. The observed CpG frequency over expected frequency can be calculated according to the method provided in Gardiner-Garden & Frommer 1987 (J Mol Biol 196:261-281). For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands. It will be appreciated though that other sequences in the human genome are prone to DNA methylation such as CpA and CpT (see Ramsahoye 2000, Proc Natl Acad Sci USA 97:5237-5242; Salmon and Kaye 1970, Biochim Biophys Acta 204:340-351; Grafstrom 1985, Nucleic Acids Res 13:2827-2842; Nyce 1986, Nucleic Acids Res 14:4353-4367; Woodcock 1987, Biochem Biophys Res Commun 145:888-894).

One embodiment relates to a method for predicting graft fibrosis in a patient eligible for receiving an allograft, or in a patient that received the allograft (i.e. to allow treatment in a later stage), comprising the steps of: determining the DNA methylation level of a CpG panel, said panel comprising at least 4 CpGs from the list shown in Table 4, in a sample of said allograft; calculating a MRS via the sum of methylation values of each CpG in said panel; comparing said MRS with the MRS of a population of reference allograft organs; and attributing a higher risk of developing graft fibrosis when the MRS is at least two-fold higher as compared to the MRS of the lower tertile of the reference population. Although not yet routinely implemented, longitudinal surveillance biopsies post-transplant are being used as monitoring tool in some clinics for detection of often unsuspected graft injury such as to adjust post-transplant treatment and to individualize therapy in order to limit allograft injury (Henderson et al. 2011, Am J Transplant 11:1570-1575). In the clinical unit of Henderson et al. (ibidem), surveillance biopsies led to change in management in 56% of their patients. In fact, one of the cohorts underlying the current invention is such a longitudinal cohort.

Another embodiment discloses a method for determining the DNA methylation level in an allograft, comprising the steps of measuring the DNA methylation of a CpG panel in a sample of the allograft, wherein said CpG panel comprises at least 4 CpGs are from the list of CpGs shown in Table 4, wherein Table 4 contains 29 CpGs with the highest reoccurrence in the Lasso models used for ranking of the importance of the CpGs identified on a genome-wide basis to predict the risk of developing renal chronic allograft injury (see Example 7). As used herein, the terms “determining”, “detecting”, “measuring,” “assessing,” and “assaying” are used interchangeably and include both quantitative and qualitative determinations. Said method for DNA methylation level determination can be a method performed in a genome-wide approach, as exemplified in the working examples, and can be any method known by a skilled person to measure the methylation level of DNA on a certain number of CpGs in a sample. The term “methylation assay” refers to any assay for determining the methylation state of one or more CpX (wherein X can be G, A, or T) dinucleotide sequences within a sequence of a nucleic acid. Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62: 503-514). As used herein, a methylation-specific reagent, refers to a compound or composition or other agent that can change or modify the nucleotide sequence of a nucleic acid molecule, a nucleotide of or a nucleic acid molecule, in a manner that reflects the methylation state of the nucleic acid molecule.

Methods of treating a nucleic acid molecule with such a reagent can include contacting the nucleic acid molecule with the reagent, coupled with additional steps, if desired, to accomplish the desired change of nucleotide sequence. In one embodiment, such a reagent modifies an unmethylated selected nucleotide to produce a different nucleotide. In another exemplary embodiment, such a reagent can deaminate unmethylated cytosine nucleotides. An exemplary reagent is bisulfite. Bisulfite genomic sequencing was recognized as a revolution in DNA methylation analysis based on conversion of genomic DNA by using sodium bisulfite. Besides various merits of the bisulfite genomic sequencing method such as being highly qualitative and quantitative, it serves as a fundamental principle to many derived methods to better interpret the mystery of DNA methylation (Li and Tollefsbol, 2011. Methods Mol Biol. 791:11-21). The most frequently used method for analyzing a nucleic acid for the presence of 5-methylcytosine is based upon the bisulfite method for the detection of 5-methylcytosines in DNA (Frommer et al. 1992, Proc Natl Acad Sci USA 89:1827-1831) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uricil is desulfonated under alkaline conditions to form uracil. Detection is possible because uracil forms base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg & Clark 1994, Bioessays 16:431-36; Grigg 1996, DNA Seq 6: 189-198) or methylation-specific PCR (MSP) as is disclosed, e.g., in U.S. Pat. No. 5,786,146.

In one embodiment, the method for determining the DNA methylation level in an allograft sample comprises treating DNA from the sample with a methylation-specific reagent, refers to treatment of DNA from the sample with said reagent for a time and under conditions sufficient to convert unmethylated DNA residues, thereby facilitating the identification of methylated and unmethylated CpG dinucleotide sequences. As used herein, the term “bisulfite reagent” refers to a reagent comprising in some embodiments bisulfite (or bisulphite), disulfite (or disulphite), hydrogen sulfite (or hydrogen sulphite), or combinations thereof to distinguish between methylated and unmethylated cytidines, e.g., in CpG dinucleotide sequences. Methods of bisulfite conversion/treatment/reaction are known in the art (e.g. WO2005038051). The bisulfite treatment can e.g. be conducted in the presence of denaturing solvents (e.g. in concentrations between 1% and 35% (v/v)) such as but not limited to n-alkylenglycol or diethylene glycol dimethyl ether (DME), or in the presence of dioxane or dioxane derivatives. The bisulfite reaction may be carried out in the presence of scavengers such as but not limited to chromane derivatives. The bisulfite conversion can be carried out at a reaction temperature between 30° C. and 70° C., whereby the temperature may be increased to over 85° C. for short times. The bisulfite treated DNA may be purified prior to the quantification. This may be conducted by any means known in the art, such as but not limited to ultrafiltration, e.g., by means of Microcon columns (Millipore). Bisulfite modifications to DNA may be detected according to methods known in the art, for example, using sequencing or detection probes which are capable of discerning the presence of a cytosine or uracil residue at the CpG site. The choice of specific DNA methylation analysis methods depends on the purpose and nature of the analysis, and is for example outlined in Kurdyukov and Bullock (2016. Biology, 5: 3).

An alternative embodiment discloses a method for predicting development of chronic allograft injury in a patient eligible for receiving an allograft, comprising the steps of:
determining the DNA methylation level of at least 4 CpGs from the list shown in Table 4, in a sample of said allograft, and in a population of reference organs;
determining the patient to be at risk of developing chronic allograft injury when DNA methylation level of the at least 4 CpGs is increased in the allograft.

The increase in the DNA methylation level can for instance refer to a value that is at least 20% higher, or at least 30% higher, or at least 50% higher, or at least 70% higher, or at least 80% higher, or at least 90% higher, or more than 100% higher, or at least 2-fold, or at least 3-fold, or more than 4-fold higher than the methylation level of the reference allograft organs, or more specifically than the methylation level of the lower tertile of the reference allograft organ population.

Another method for predicting development of chronic allograft injury in a patient eligible for receiving an allograft, comprises the steps of:
determining the DNA methylation level of at least 4 CpGs from the list shown in Table 4, in a sample of said allograft,
comparing the DNA methylation level of the at least 4 CpGs with the DNA methylation level of the same at least 4 CpGs in a population of reference organs,
determining the patient to be at risk of developing chronic allograft injury when the DNA methylation level of the at least 4 CpGs is at least two-fold higher as compared to the lower tertile of the reference population.

In a number of embodiments, the DNA methylation level is used to calculate the methylation risk score, which is compared to one or more control MRS values. A “methylation risk score”, “DNA methylation score”, “risk score”, or “methylation score”, as used interchangeably herein, may be developed and/or calculated via several formulas, and is based in the methylation level or value of a number of CpGs. One example of a method for MRS calculation is provided by Ahmad et al. (2016. Oncotarget, 7(44):71833) being developed from the multivariate Cox model. Another MRS calculation method as used herein is explained in the section “Statistical Analysis” of the Methods as applied in the Examples. A person skilled in the art will be aware of applicable formulas and models for implementation and development of the MRS of the present method of the invention. Once the MRS is obtained for an allograft sample, the prediction of the outcome or higher risk of developing CAI is dependent on a comparison of said MRS to a reference population, or the MRS of a reference population, or the average or mean MRS of a reference population. Said reference population comprises allograft samples from a population of subjects with a mixtures of high and low MRS values, representing healthy high-quality and damaged low-quality allografts or donor organs, which can be ranked and classified according to the MRS value. The part of the population with the highest MRS were demonstrated to have a CADI>2, indicating CAI outcome at 1 year. Finally, the method of the present invention attributes or predicts a higher risk of developing CAI when the MRS of the allograft sample is at least two-fold higher as compared to the lowest tertile of the reference population.

The prediction or attribution of a ‘higher risk’ for CAI or ‘higher risk’ of developing CAI is defined herein as an increase of at least 9-fold higher risk (see Example 6). In another embodiment the prediction of outcome for a higher risk for CAI involved an increase or higher risk of at least 5-fold, 6-fold, 7-fold or 8-fold as compared to the lowest tertile of the reference population.

In one embodiment, the method of the present invention attributes or predicts a higher or increased risk of developing CAI when the MRS is “higher” as compared to the lower tertile of the reference population, wherein “a higher MRS” is defined as at least 2-fold higher as compared to the MRS of the lower or lowest tertile of the reference population, or the average or mean of the MRS of the reference population. In some embodiments, the “higher MRS” is defined as at least 3-fold, 4-fold or 5-fold higher as compared to the MRS of the lower or lowest tertile of the reference population. Alternatively, “higher MRS” for an allograft sample or for a patient eligible in receiving the allograft may also be defined as a “higher MRS as compared to the MRS of the lowest tertile of a reference population, wherein the MRS of the reference, or the average or mean of the MRS of the reference is at least 70%, 60%, 50%, 40%, 30%, 20%, or 10% of the allograft sample MRS.

The control or reference MRS may be a reference value and/or may be derived from one or more samples, also an average or mean MRS may be used, optionally from historical methylation data for a patient/allograft or pool of patients or pool of allografts. In such cases, the historical methylation data can be a value that is continually updated as further samples are collected and MRSes are defined for different allograft samples or for different patients. It will be understood that the control may also represent an average of the methylation levels or an average of the MRS for a group of samples or patients, in particular for a group of samples from organs which are the same as the allografted organ. In particular, said MRS of said sample or of said controls may be based on a calculation using selected CpG loci as described herein (i.e. derived from Table 2—66 CpG islands containing 1634 CpGs shown to be biomarkers for hypermethylation in renal CAI; or derived from Table 3 containing 413 CpGs—used in the 1000 iterative lasso's as predictive biomarkers for hypermethylation in renal CAI; or derived from Table 4, containing 29 CpGs as most frequently reoccurring CpGs in the 1000 iterative lasso's shown to be biomarkers for hypermethylation in renal CAI). Average methylation or MRS values may, for example, also include mean values or median values.

The method of the present invention in one embodiment relates to an MRS calculation based on the methylation values of the CpGs of a CpG panel, wherein said panel comprises at least 4 CpGs from the list of CpGs shown in Table 4. Any combination of at least 4 or more CpGs from said list of 29 CpGs presented in Table 4 allows calculation of the MRS to predict the risk of developing CAI wherein said prediction is outperforming or better than the current clinical parameters. As non-limiting examples, a combination of at least 4 CpGs from said list in Table 4 for calculation of the MRS may comprise cg01811187, cg17078427, cg16547027, and cg19596468; alternatively another combination may comprise cg01811187, cg14309111, cg17603502, and cg08133931; alternatively another combination may comprise cg17078427, cg14309111, cg17603502, and cg08133931; alternatively another combination may comprise cg16547027, cg14309111, cg17603502, and cg08133931; among other combinations. Further non-limiting examples of combinations of 4 CpGs of Table 4 wherein at least one of the CpGs is cg01811187, is cg17078427, is cg16547027, is cg19596468, is cg14309111, is cg17603502, is cg08133931, is cg18599069, is cg24840099, is cg09529433, is cg10096645, is cg06108383, is cg03884082, is cg01065003, is cg22647713, is cg20449692, is cg07136023, is cg20811659, is cg20048434, is cg06546607, is cg00403498, is cg20891301, is cg17416730, is cg01724566, is cg16501308, is cg06230736, is cg03199651, is cg06329022, or is cg13879776. Certain combinations of at least 4CpGs selected from Table 4 may also relate to a combination that includes all CpGs of Table 4 relating to the same reference gene, such as the combination of cg19596468, cg24840099, cg20891301, and cg03199651 all referring to MSX1, or the combination of cg01811187, cg09529433, cg20811659, all referring to CACNA1G, in combination with all CpGs referring to another gene, for instance KCTD1, for cg16547027, cg10096645, and cg01065003. Another combination such as cg17078427, cg20449692, cg13879776, all referring to the gene CLDN11, in further combination with another CpG(s) listed in the Table 4 is also possible. In fact, also a combination of at least all CpGs present in table 4 relating to at least 4 gene names may also be in the scope of the CpG panel for the method of the invention, non-limiting examples being provided for in a combination of all CpGs for CACNA1G, CLDN11, KCTD1 and ODZ4, resulting in cg01811187, cg09529433, cg20811659, cg17078427, cg20449692, cg13879776, cg16547027, cg10096645, cg1065003, cg14309111. Alternatively, all CpGs from Table 4 referring to ODZ4 (cg14309111), HS3ST3B1 (cg17603502), NBL1 (cg03884082), and AFAP1L2 (cg20048434) may be sufficient as well to determine the MRS score for the method of the invention.

In another embodiment, at least 5 CpGs from said list of Table 4 is sufficient for calculation of the MRS of the method of the invention. In alternative embodiments, the CpG panel of the present method relates to at least 6, 7, 8, 9, 10, 11, 12, 13, 14, 15, 16, 17, 18, 19, 20, 21, 22, 23, 24, 25, 26, 27, or 28 CpGs to determine the methylation level from, and use for the development of the MRS score for prediction of the risk of developing CAI in a patient eligible for receiving an allograft. An alternative embodiment relates to the CpG panel of the present method consisting of a maximum of 4 CpGs selected from said list of 29 CpGs presented in Table 4, to determine the methylation level from, and to use for the development of the MRS score for prediction of the risk of developing CAI in a patient eligible for receiving an allograft. Further alternative embodiments relate to the CpG panel of the present method consisting of a maximum of 5, 6, 7, 8, 9, 10, 11, 12, 13, 14, 15, 16, 17, 18, 19, 20, 21, 22, 23, 24, 25, 26, 27, or 28 CpGs from said list of 29 CpGs presented in Table 4, to determine the methylation level from, and to use for the development of the MRS score for prediction of the risk of developing CAI, in particular for graft fibrosis, in a patient eligible for receiving an allograft. In alternative embodiments, all provided that at least 4 CpGs of Table 4 are included, the panel of CpGs is consisting of a maximum of (up to) 413 CpGs of Table 3, is consisting of a maximum of (up to) 1634 CpGs of Table 2, is consisting of a maximum of between 29 and 413 CpGs (of Table 3), is consisting of a maximum of between 29 and 1634 CpGs (of Table 2), is consisting of a maximum of between 413 CpGs (of Table 3) and 1634 CpGs (of Table 2), or is consisting of a maximum of 5, 6, 7, 8, 9, 10, 11, 12, 13, 14, 15, 16, 17, 18, 19, 20, 25, 30, 35, 40, 45, 50, 60, 70, 80, 90, or 100 CpGs (wherein the CpGs not taken from Table 4 are taken from Tables 2 or 3).

Moreover, an embodiment relates to the method of the present invention in which the CpG panel comprises the 29 CpGs listed in Table 4. Another embodiment relates to the method of the present invention in which the CpG panel comprises a number of CpGs listed in Table 4, wherein the CpG annotated on a particular gene within said Table 4 is not included in said CpG panel. As a non-limiting example, in one embodiment the method of the present invention comprises a CpG panel consisting of 26 CpGs of Table 4, wherein the CpGs annotated to the GATA3 gene are for instance excluded. In another embodiment the method of the present invention comprises the CpG panel of the 413 CpGs listed in Table 3. Another embodiment relates to the method of the present invention in which the CpG panel comprises the 1634 CpGs listed in Table 2, namely the identified CpGs being methylated in the validated 66 CpG islands, as presented in Table 2.

Moreover, an embodiment relates to the method of the present invention in which the CpG panel consists of the 29 CpGs listed in Table 4. Another embodiment relates to the method of the present invention in which the CpG panel consists of a number of CpGs listed in Table 4, wherein the CpG annotated on a particular gene within said Table 4 is not included in said CpG panel. As a non-limiting example, in one embodiment the method of the present invention consists of a CpG panel of 26 CpGs of Table 4, wherein the CpGs annotated to the GATA3 gene are for instance excluded. In another embodiment the method of the present invention consists the CpG panel of the 413 CpGs listed in Table 3. Another embodiment relates to the method of the present invention in which the CpG panel consists of the 1634 CpGs listed in Table 2, namely the identified CpGs being methylated in the validated 66 CpG islands, as presented in Table 2.

Alternatively, the methylation β values (as an estimate of methylation level using the ratio of intensities between methylated and unmethylated alleles. β values range between 0 and 1, with β=0 being unmethylated and β=1 being fully methylated), are calculated or determined by a skilled person, in the method of the invention, for at least 4 CpGs of the CpGs listed herein (in Table 4), to predict the risk for developing CAI. In one embodiment, a method for predicting development of chronic allograft injury in a patient eligible for receiving an allograft, comprises the steps of:
determining the DNA methylation β values of at least 4 CpGs from the list shown in Table 4, in a sample of said allograft, and in a population of reference organs;
determining the patient to be at risk of developing chronic allograft injury when DNA methylation β values of each of the at least 4 CpGs is increased in the allograft.

In another embodiment, the method for predicting development of chronic allograft injury in a patient eligible for receiving an allograft, comprises the steps of:
determining the DNA methylation β values of at least 4 CpGs from the list shown in Table 4, in a sample of said allograft;
determining the patient to be at risk of developing chronic allograft injury when DNA methylation β values of each of the at least 4 CpGs is increased in the allograft compared to reference organs or compared to the lower tertile of the reference organs.

The method relating to said determination of DNA methylation β values of each of the at least 4 CpGs in fact indicates an increased risk of developing chronic allograft injury when those β values are at least 0.025 higher in the allograft as compared to the control or reference.

Alternatively, said 3 values of each of the at least 4 CpGs in fact indicates an increased risk of developing chronic allograft injury are at least 0.05, at least 0.075, at least 0.1, at least 0.125, at least 0.15, at least 0.175, at least 0.2, at least 0.2125, at least 0.225, at least 0.25, at least 0.275, at least 0.3, at least 0.325, at least 0.35, or at least 0.375 higher in the allograft as compared to the control or reference.

Another embodiment relates to a method for predicting or determining (development of) (renal) allograft fibrosis and/or chronic allograft injury in a sample obtained from a subject, the method comprising:
assaying a methylation state of at least four CpG markers in a sample obtained from a subject; and
identifying the subject as having a higher risk of developing allograft fibrosis and/or chronic allograft injury when the methylation state of the at least four CpG markers is different than a methylation state of the at least 4 CpG markers assayed in a subject that does not have a high risk of developing allograft fibrosis or injury, or has no transplant kidney (i.e. a renal biopsy from a healthy person), wherein the at least four CpG markers comprise a base in a differentially methylated region (DMR) selected from a group consisting of CpGs in Table 4, or in Table 3, or in Table 6, or in Table 2.

Another alternative method for characterizing a biological sample from an allograft relates to a method comprising the steps of:
measuring a methylation level of a CpG site for one or more genes selected from the list of genes in Table 4 in a biological sample of a human individual through treating genomic DNA in the biological sample with bisulfite; and amplifying the bisulfite-treated genomic DNA using gene-specific primers for the selected one or more genes and determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
comparing the methylation level to a methylation level of a corresponding set of genes in control samples without predicted allograft injury (or wild-type normal samples that did not undergo transplantation); and
determining that the individual has higher risk of developing allograft fibrosis and/or chronic allograft injury when the methylation level measured in the one or more genes is higher than the methylation level measured in the respective control samples. With biological sample is meant a biopsy sample from an allograft or transplant organ, which may be a liquid biopsy. The CpG sites for one or more genes comprise at least 4 CpGs in a particular embodiment.

Another embodiment discloses a method for measuring the methylation level of at least 4 or more CpG sites listed in Table 4 comprising:
extracting genomic DNA from a biological sample of a human individual suspected of having or having allograft fibrosis or chronic allograft injury,
treating the extracted genomic DNA with bisulfite,
amplifying the bisulfite-treated genomic DNA with primers consisting of a pair of primers specific for any of the genes listed in Table 4, and
measuring the methylation level of one or more CpG sites listed in Table 4 by methylation-specific PCR, quantitative methylation-specific PCR, methylation sensitive DNA restriction enzyme analysis or bisulfite genomic sequencing PCR.

In any of these methods, any of the CpG panels described in detail hereinabove can be applied.

Assays for DNA methylation analysis have been reviewed by e.g. Laird 2010 (Nat Rev Genet 11:191-203). The main principles of possible sample pretreatment involve enzyme digestion (relying on restriction enzymes sensitive or insensitive to methylated nucleotides), affinity enrichment (involving e.g. chromatin immunoprecipitation, antibodies specific for 5MeC, methyl-binding proteins), sodium bisulfite treatment (converting an epigenetic difference into a genetic difference) followed by analytical steps (locus-specific analysis, gel-based analysis, array-based analysis, next-generation sequencing-based analysis) optionally combined in a comprehensible matrix of assays. Laird 2010 is providing a plethora of bioinformatic resources useful in DNA methylation analysis which can be applied by the skilled person as guiding principles, when wishing to analyze the methylation status of up to about 100 CpGs in a sample, with assays such as MethyLight, EpiTYPER, MSP, COBRA, Pyrosequencing, Southern blot and Sanger BS appearing to be the most suitable assays. This guidance does, however, not take into account that assays with higher coverage can be adapted towards lower coverage. For example, design of custom DNA methylation profiling assays covering up to 96 or up to 384 individual regions is possible e.g. by using the VeraCode® technology provided by Illumina® (compared to the 450K DNA methylation array covering approximately 480000 individual CpGs). Another such adaptation for instance is enrichment of genome fractions comprising methylation regions of interest which is possible by e.g. hybridization with bait sequences. Such enrichment may occur before bisulfite conversion (e.g. customized version of the SureSelect Human Methyl-Seq from Agilent) or after bisulfite conversion (e.g. customized version of the SeqCap Epi CpGiant Enrichment Kit from Roche). Such targeted enrichment can be considered as a further modification/simplification of RRBS (Reduced Representation Bisulfite Sequencing).

The MethyLight assay is a high-throughput quantitative or semi-quantitative methylation assay that utilizes fluorescence-based real-time PCR (e.g., TagMan®) that requires no further manipulations after the PCR step (Eads et al. 2000, Nucleic Acids Res 28:e32). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs at the level of the amplification process, at the level of the probe detection process, or at both levels. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites or with oligonucleotides covering potential methylation sites.

The EpiTYPER assay involves many steps including gene-specific amplification of bisulfite-converted genomic DNA, in vitro transcription of the amplified DNA, uranil-specific cleavage of transcribed RNA, and MALDI-TOF analysis of the RNA fragments. The EpiTYPER software finally distinguishes between methylated and non-methylated cytosine in the genomic DNA.

Methylation-specific PCR (MSP) refers to the methylation assay as described by Herman et al. 1996 (Proc Natl Acad Sci USA 93:9821-9826), and by U.S. Pat. No. 5,786,146. MSP (methylation-specific PCR) allows for assessing the methylation status of virtually any group of CpG sites within a CpG island, independent of the use of methylation-sensitive restriction enzymes. Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG. Variations of MSP include Methylation-sensitive Single Nucleotide Primer Extension (Ms-SNuPE; Gonzalgo & Jones 1997, Nucleic Acids Res 25:2529-2531). Another variation, however including restriction enzyme digestion instead of bisulfite modification as sample pretreatment, is Methylation-Sensitive Arbitrarily-Primed Polymerase Chain Reaction (MS AP-PCR; Gonzalgo et al. 1997, Cancer Research 57:594-599).

Combined Bisulfite Restriction Analysis (COBRA) refers to the methylation assay described by Xiong & Laird 1997 (Nucleic Acids Res 25:2532-2534). COBRA analysis is a quantitative methylation assay useful for determining DNA methylation levels at specific loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by bisulfite treatment. PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Sanger BS is the original way of analysis of bisulfite-treated DNA: gel electrophoresis-based Sanger sequencing of cloned PCR products from single loci (Frommer et al. 1992, Proc Natl Acad Sci USA 89:1827-1831). A technique such as pyrosequencing is similar to Sanger BS and obviates the need of gel electrophoresis; it, however, requires other specialized equipment (e.g. Pyromark instrument). Sequencing approaches are still applied, especially with the emergence of next-generation sequencing (NGS) platforms. Southern blot analysis of DNA methylation depends on methyl-sensitive restriction enzymes (e.g. Moore 2001, Methods Mol Biol 181:193-201).

Other assays to determine CpG methylation include the HeavyMethyl (HM) assay (Cottrell et al. 2004, Nucleic Acids Res 32, e10; WO2004113567), Methylated CpG Island Amplification (MCA; Toyota et al. 1999, Cancer Res 59:2307-12; WO 00/26401), Reduced Representation Bisulfite Sequencing (RRBS; e.g. Meissner et al. 2005, Nucleic Acids Res 33: 5868-5877), Quantitative Allele-specific Real-time Target and Signal amplification (QuARTS; e.g. WO2012067830), and assays described in Laird et al. 2010 (Nat Rev Genet 11:191-203) and in Kurdyukov & Bullock 2016 (Biology 5(1), pii: E3).

“Ischemia” is a vascular phenomenon caused by obstruction of blood flow to a tissue, for instance as a result from vasoconstriction, thrombosis or embolism, resulting in limited supply of oxygen and nutrients, and if prolonged, in impairment of energy metabolism and cell death. Restoration of the blood flow, called “Reperfusion”, results in oxygen reintroduction and a burst of ROS, leading to cell death associated with inflammation (Jouan-Lanhouet et al., 2014; Vanlangenakker et al., 2008; Halestrap, 2006). Ischemia can occur acutely, as during surgery, or from trauma to tissue incurred in accidents, injuries and war setting, or following harvest of organs intended for subsequent transplantation, for example. It can also occur sub-acutely, as found in atherosclerotic peripheral vascular disease, where progressive narrowing of blood vessels leads to inadequate blood flow to tissues and organs. If ischemia is ended by the restoration of blood flow, a second series of injuries events ensue, producing additional injury. Thus, whenever there is a transient decrease or interruption of blood flow in a subject, the resultant injury involves two-components, the direct injury occurring during the ischemic interval, and the indirect or reperfusion injury that follows, therefore named “Ischemia-Reperfusion Injury (IRI)”. Current understanding is that much of this injury is caused by chemical products, free radicals, and active biological agents released by the ischemic tissues.

In some embodiments of the method of the present invention, the allograft is a kidney or the allograft sample is a renal biopsy, or renal tissue. Basically two ways to perform a renal biopsy exist: percutaneous biopsy (renal needle biopsy) and open biopsy (surgical biopsy). The percutaneous biopsy is most common and employs a thin biopsy needle to remove kidney tissue wherein the needle may be guided using ultrasound or CT scan. For small renal tissue samples, a fine needle aspiration biopsy is possible, whereas for larger renal tissue samples, a needle core biopsy is obtained by e.g. using a spring-loaded needle. Kidney or renal IR or IRI was found to be a major cause of acute kidney injury (AKI) in many clinical settings including cardiovascular surgery, sepsis, and kidney transplantation. Ischemic AKI is associated with increased morbidity, mortality, and prolonged hospitalization (Bagshaw 2006; Korkeila et al., 2000). Acute ischemia leads to depletion of adenosine triphosphate (ATP), inducing tubular epithelial cell (TEC) injury, and hypoxic cell death. Reperfusion further amplifies injury by promoting the formation of reactive oxygen species (ROS), and inducing leukocyte activation, infiltration and inflammation (Devrajan 2005; Dagher et al., 2003; Li and Jackson, 2002). Chronic allograft injury (CAI) is also very common after kidney transplantation in which immunological (e.g., acute and chronic cellular and antibody-mediated rejection) and nonimmunological factors (e.g., donor-related factors, ischemia-reperfusion injury, polyoma virus, hypertension, and calcineurin inhibitor nephrotoxicity) have a role. Despite the new Banff pathological classification, histopathological diagnosis is still far from being the ‘gold standard’ to understand the exact mechanisms in the development of CAI, which may lead to appropriate treatment (Akalin and O'Connell, 2010. Kidney International 78 (Suppl 119), S33-S37). Fibrosis and cell death may also be determined using DNA methylation detection on specific CpGs according to the current invention, since many of the induced hypermethylation was observed predominantly near genes involved in ‘negative regulation’ of fibrosis and cell death.

The method of the present invention for predicting the risk of developing allograft fibrosis and/or CAI in a patient eligible for receiving an allograft, comprising a sample of an allograft is in one embodiment represented by an allograft sample taken from a donor organ or from a patient before transplantation or implantation. In another embodiment said allograft sample is taken right after transplantation of the allograft in the receiving patient, or after a period of implantation. In one embodiment, said sample of the allograft is taken and analyzed at the time of transplantation or just prior to implantation, meaning just before the surgery, but after the preservation. Said time for sampling allows the more accurate determination of attributing a risk of developing CAI in said patient receiving said allograft, and for anticipation of post-treatment to avoid or overcome CAI due to ischemia-induced hypermethylation events that took place prior to implantation in the allograft.

Another aspect of the invention relates to an inhibitor of DNA methylation or hypermethylation, for use in preservation of the allograft prior to implantation or transplantation, wherein a higher risk of developing chronic allograft injury in a patient was predicted for said allograft, according to the method for determining CpG methylation levels described herein. In fact, a sample of the allograft should be taken at the time of implantation, for determining the CpG methylation level. In fact, when using a kit of the invention (see further), or of, e.g., a further developed chip based on those CpG markers, the analysis time should be as short as possible to provide for a clear insight in prediction of future allograft injury, and to preserve the allograft via the use of said inhibitor. This use in preservation or treatment of the organ, in order to hypomethylate or revert hypermethylation involves to incubate said inhibitor in suitable conditions with the allograft, or treat the allograft, which may be an organ, tissue or cells that may have suffered from ischemia-induced hypermethylation during the period between removal of the allograft from the donor and receival or implantation of the allograft in the patient. Hypermethylation is reversible, and several compounds are used as methylation inhibitors, mainly in the field of cancer and in hypoxic tumors. Non-limiting examples comprise 5-azacytidine (AZA), a cytidine analog which is used for demethylation and also approved (as Vidaza) for treatment of myelodysplastic syndrome or other cancers, and decitabine (DEC) (Licht, 2015. Cell 162: 938). Furthermore, by modulating the TET enzyme activity, compounds such as α-ketoglutarate, a cofactor of the TET enzymes, may also act in inhibiting DNA methylation under hypoxic or anoxic conditions. So in one embodiment, a stimulator of TET enzyme activity is used for preservation or treatment of the allograft prior or post transplantation, when a higher risk of developing chronic allograft injury in a patient was predicted for said allograft, according to the method for determining CpG methylation levels described herein. The TET enzyme is converting methylated cytosine (5mC) into hydroxymethylated cytosine (5hmC), a reaction which is inhibited upon oxygen shortage. So stimulation of the TET enzyme activity may also be accomplished by oxygenation. In one embodiment, a method for preservation of the allograft comprises reverting hypermethylation of CpGs in the allograft by oxygenation. In another embodiment, stimulation of TET activity is established via acting on or modulating another enzyme that affects TET activity. For instance, in one embodiment, said stimulator of TET activity for use in preservation of allograft prior to transplantation is a modulator or inhibitor of BCAT1 activity. In fact, BCAT activity results reversible transamination of an α-amino group from branched-chain amino acids (BCAAs; i.e. valine, leucine and isoleucine) to α-ketoglutarate (aKG), which is a critical regulator of its own intracellular homeostasis and essential as cofactor for aKG-dependent dioxygenases such as the TET enzyme family (Raffel et al., 2017. Nature, 551: 384). By reducing the activity of BCAT1, intracellular aKG levels increase, thereby stimulating TET, resulting in inhibition of 5mC formation or DNA methylation. Recently, the role of BCAT1 in macrophages has been investigated, and the BCAT1-specific inhibitor, ERG240, a leucine analogue, showed reduced inflammation through a decrease of macrophage infiltration in for instance kidneys (Papathanassia et al., 2017. Nat. communic. 8: 16040). These findings all together allow to conclude that such BCAT1 inhibitors represent an alternative in the treatment needed to preserve allografts, via a mechanism acting on inhibition of hypermethylation.

In a specific embodiment, an inhibitor of hypermethylation or a stimulator of TET enzyme activity is used to preserve the allograft prior to implantation, especially for said allografts for which a higher risk of developing CAI in the receiving patient has been predicted. In fact, the method of the present invention for predicting the risk of developing CAI may be used to determine which are those allografts.

Alternative embodiments relate to an inhibitor of hypermethylation or a stimulator of TET enzyme activity for use in preservation of the allograft prior to implantation, to prevent chronic allograft injury in a patient, in particular in a patient eligible for receiving said allograft.

In a specific embodiment, said inhibitor of hypermethylation or a stimulator of TET enzyme activity for use in preservation of the allograft prior to implantation, in particular inhibits or reverts the methylation of those CpGs that are hallmarks in the present invention to predict for a higher risk of developing CAI, as referred to in Table 4.

In some embodiments, said inhibitor of hypermethylation or a stimulator of TET enzyme activity is for use in preservation of the allograft prior to implantation. In some embodiments, said inhibitor of hypermethylation or a stimulator of TET enzyme activity is for administering to or treatment of a patient that received said allograft, so after implantation, and wherein a higher risk of developing chronic allograft injury in a patient was predicted for said allograft, according to the method for determining CpG methylation levels described herein. In another embodiment, a composition or pharmaceutical composition of said inhibitor of hypermethylation or stimulator of TET activity for use in preservation of the allograft prior to implantation is used. Alternatively, a composition or pharmaceutical composition of said inhibitor of hypermethylation or stimulator of TET activity is used for administration to or treatment of a patient, or for use as a medicament, after determination of the CpG methylation levels according to the method described herein, and attributing a higher risk of developing graft fibrosis or CAI.

Other embodiments relate to the method of the invention, comprising the steps of: determining the DNA methylation level of a CpG panel in a sample of said allograft, calculating an MRS for said CpG panel, comparing the MRS of the sample of the allograft with a reference population of allografts, and attributing a higher risk of developing chronic allograft injury when the MRS is at least two-fold higher as compared to the lower tertile of the reference population, further comprising the step of preservation of the allograft to prevent or inhibit CAI. Alternatively, embodiments relate to said method of the invention, further comprising the step of preservation of the allograft to prevent or inhibit CAI, wherein said preservation is established by using an inhibitor or hypermethylation or a stimulator of TET activity. Alternatively, embodiments relate to said method of the invention, further comprising the step of treatment of the patient or recipient to prevent or inhibit CAI in said patient. In a preferred embodiment, said allograft being a kidney. Another embodiment relates to said method, further comprising a treatment comprising adaptive treatment in comparison to the standard post-implantation treatment of the recipient. Moreover, the method of the invention may be used on a biopsy sample taken after a certain period post-transplantation, and upon outcome of a higher risk of developing CAI, the appropriate treatment, being administration of inhibitors of methylation, stimulators of TET activity, specific methods for local oxygenation, among others, may be applied to revert and further prevent chronic injury or graft rejection or kidney failure.

The term “composition” or “pharmaceutical compositions” relates to one or more compounds of the invention, in particular, the inhibitor of hypermethylation or a stimulator of TET enzyme activity and a pharmaceutically acceptable carrier or diluent, for use in preservation of the allograft. These pharmaceutical compositions can be utilized to achieve the desired pharmacological effect by administration to an allograft or to the patient receiving the allograft. The present invention includes pharmaceutical compositions that are comprised of a pharmaceutically acceptable carrier and a pharmaceutically effective amount of a compound, or salt thereof, of the present invention, for use in preservation of the allograft prior to implantation. A pharmaceutically effective amount of compound is preferably that amount which produces a result or exerts an influence on the particular condition being treated. In general, “therapeutically effective amount”, “therapeutically effective dose” and “effective amount” means the amount needed to achieve the desired result or results. One of ordinary skill in the art will recognize that the potency and, therefore, an “effective amount” can vary depending on the identity and structure of the compound of the invention. One skilled in the art can readily assess the potency of the compound. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvants typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)—Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171). The term “excipient” is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

Another aspect of the invention relates to the use of a panel of CpGs for prediction of the risk of developing allograft fibrosis and/or CAI, wherein said CpG panel comprises at least 4 CpGs from the list of CpGs in Table 4, or wherein said CpG panel is any of the CpG panels as described in detail hereinabove. Alternatively, a panel of CpGs may be used in a method for prediction of the risk of developing allograft fibrosis and/or CAI, wherein said CpG panel comprises at least 4 CpGs from the list of CpGs in Table 4, or wherein said CpG panel is any of the CpG panels as described in detail hereinabove. The term ‘biomarker’, ‘biomarker panel’, ‘panel of CpGs’, or ‘CpG panel’ as referred to herein relates to means that specifically detect those specific CpGs referred to. Said biomarker panel of CpGs herein refers to predictive biomarkers which upon detection of alteration in their methylation status indicated the increased risk of developing allograft fibrosis and/or CAI. In an alternative embodiment, said CpG panel comprises the 29 CpGs as listed in Table 4, or said CpG panel comprises the 413 CpGs as listed in Table 3, or said CpG panel comprises the 1238 CpGs as listed in Table 6, or said CpG panel comprises the 1634 CpGs as listed in Table 2, which contains the 66 CpG islands validated to relate to hypermethylated CpGs hallmarking a higher risk of developing CAI. A specific embodiment relates to the use of said biomarker CpG panel for predicting the risk of developing CAI, wherein the allograft is kidney. In a specific embodiment, the invention relates to a method for methylation level analysis of at least 4 CpG biomarkers from the list consisting of Table 4. In particular, the prediction of the risk of developing allograft fibrosis and/or CAI is performed according to any of the methods described hereinabove.

In a final aspect of the invention, a kit for determining the DNA methylation level of a CpG panel is disclosed, wherein said kit comprises one or more reagents to measure the methylation level of DNA, specifically for at least 4 CpGs from the list in Table 4, or for any of the CpG panels as described in detail hereinabove. Envisaged kit reagents are for instance primers and/or probes (optionally provided on a solid support; one of the primers or probes provided may comprise a detectable label) targeting the CpGs of the intended CpG panel, and/or a bisulfite reagent. The kit may also comprise an insert or leaflet with instructions on how to operate the kit. In particular, the kit is used in or for use in a method of prediction of the risk of developing allograft fibrosis and/or CAI, wherein the method is any of the methods described hereinabove. One embodiment relates to the use of said kit for determining the methylation level of at least 4 CpGs from a list consisting of the CpGs in Table 4. A more specific embodiment relates to the use of said kit further comprising primers and/or probes for detecting the methylation levels from the at least 4 biomarker CpGs, and in an even more specific embodiment at least one of the primers and/or probes comprises a label. Specific embodiments relate to the use of said kit, further comprising an artificially generated methylation standard. In some embodiments, the kit further comprises bisulfite conversion reagents, methylation-dependent restriction enzymes, methylation-sensitive restriction enzymes, and/or PCR reagents.

In one embodiment, the use of said kit of the invention in a method of the present invention is aimed for. In particular, the use of said kit for predicting the risk of developing CAI in a patient. In a preferred embodiment, the use of said kit for predicting the risk of developing renal CAI in a patient eligible for receiving said allograft, in particular, said donor kidney is disclosed. In another embodiment, the use of said kit further comprises a post-ischemia sample.

In an embodiment, the kit further comprises a computer-readable medium that causes a computer to compare methylation levels from a sample at the selected CpG loci to one or more control or reference profiles and computes an MRS or correlation value between the sample and control profile. In an embodiment, the computer readable medium obtains the control or reference profile from historical methylation data for an allograft or patient or pool of allografts or patients known to have, or not have, undergone ischemia for transplantation. In some embodiments, the computer readable medium causes a computer to update the control or reference based on the testing results from the testing of a new allograft sample.

It is to be understood that although particular embodiments, specific configurations as well as materials and/or molecules, have been discussed herein for engineered cells and methods according to the present invention, various changes or modifications in form and detail may be made without departing from the scope of this invention. The following examples are provided to better illustrate particular embodiments, and they should not be considered limiting the application. The application is limited only by the claims.

### EXAMPLES

### Example 1. DNA Hypermethylation of Kidney Allografts Following Ischemia

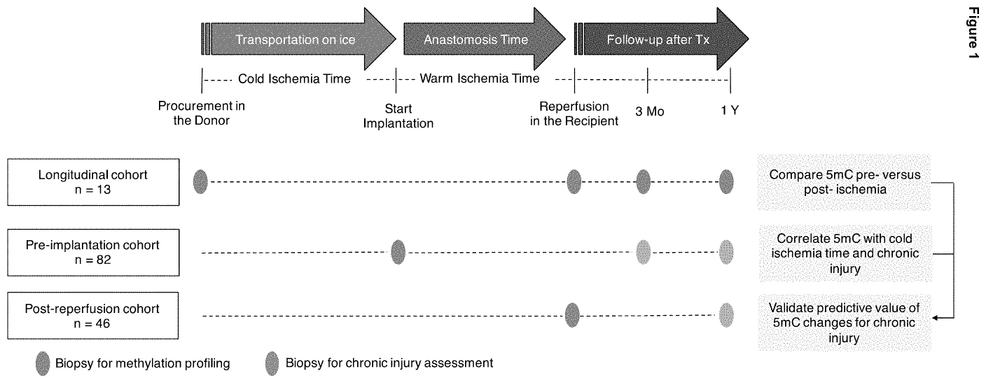

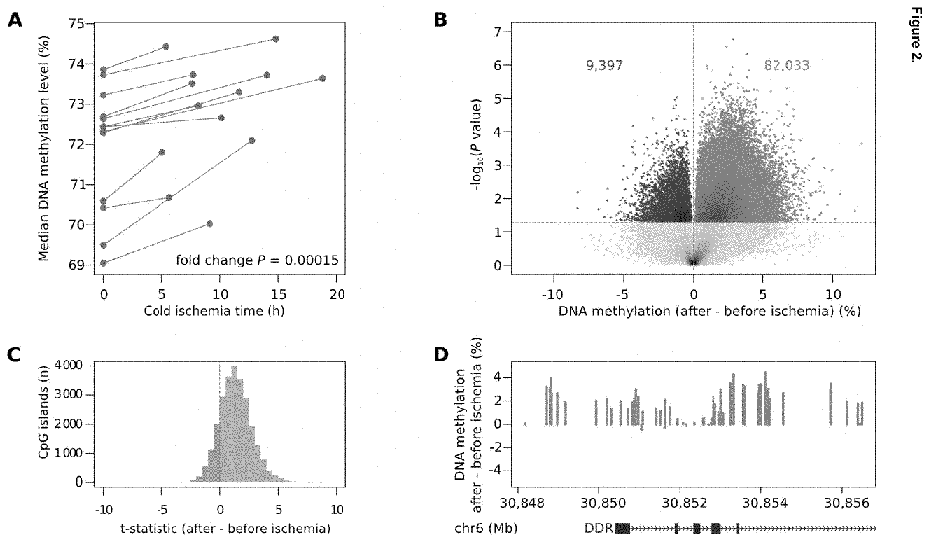

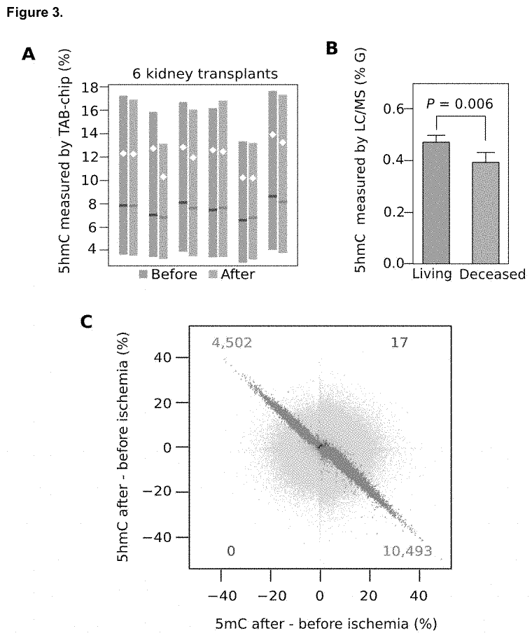

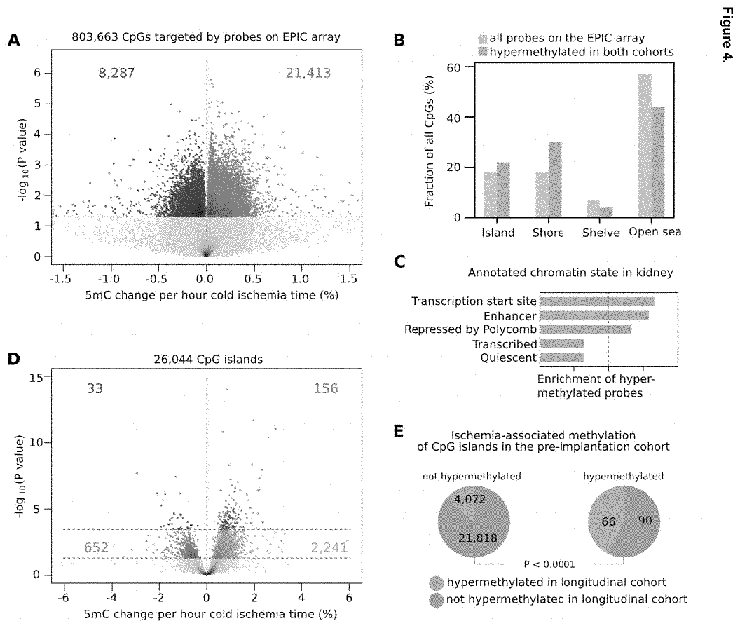

To evaluate DNA methylation changes arising during cold ischemia, we set up a prospective clinical study to collect paired pre-ischemic procurement and post-ischemic reperfusion biopsies of 13 brain-dead donor kidney transplants (FIG. 1). This paired design minimized inter-individual differences, such as genetic differences, age and gender, which are known to profoundly influence DNA methylation levels. The average cold ischemia time was 10.1±4.1 hours. Table 1 summarizes the other donor, transplant and recipient characteristics.

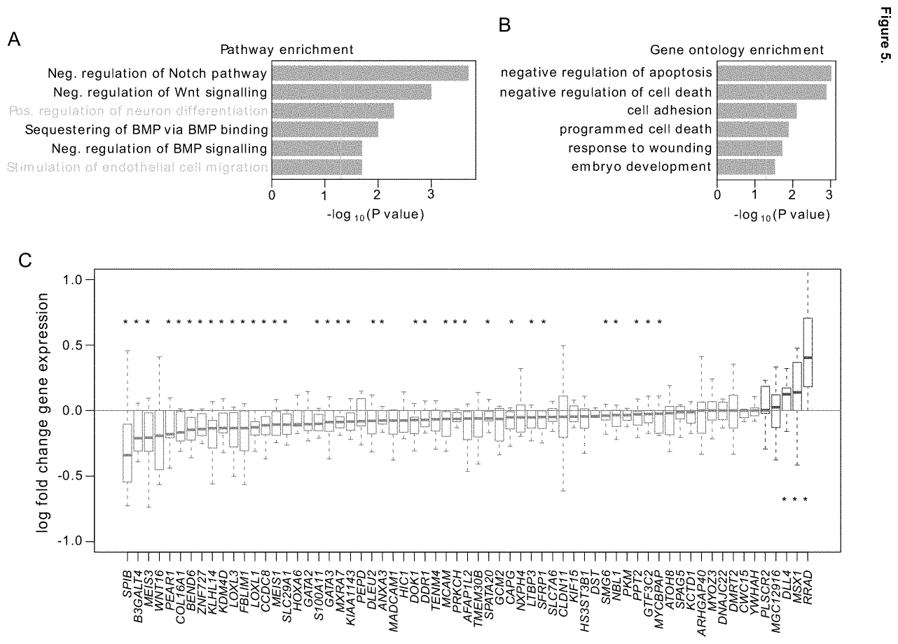

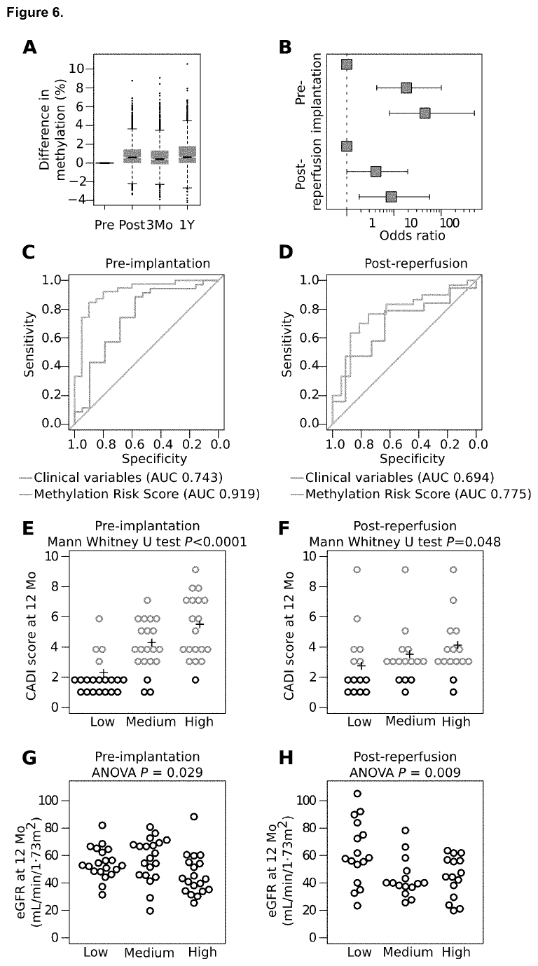

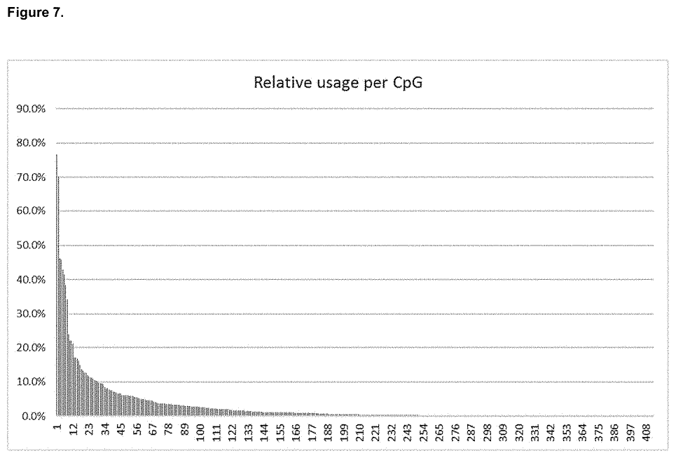

DNA methylation levels were analysed for >850,000 CpGs using Illumina EPIC beadchips micro-arrays¹⁰ and, following normalisation, pre- versus post-ischemia levels were compared in a pair-wise fashion. First, we evaluated global DNA methylation levels averaged across all probes. We observed an increase in each transplant pair following ischemia (median increase: 1.3±0.9%, P=0.0002, FIG. 2A). Next, we assessed which individual CpGs were affected by ischemia. We identified 91,430 differentially methylated sites (P<0.05), most of which showed hypermethylation in the post-reperfusion biopsy (82,033 CpG sites, 90%; P<0.00001, FIG. 2B). Methylation levels of these CpGs increased up to 12.1% after ischemia. Significantly hypermethylated CpGs were frequently found near CpG islands, particularly within CpG island shores (20.2% versus 17.8% by random chance, P<0.00001). We therefore grouped methylation of individual CpGs per CpG island: the vast majority of CpG islands (22,001 out of 26,046, 84.5%) were hypermethylated after ischemia (FIG. 2C), of which 8,018 at P<0.05. When correcting for multiple testing (FDR<0.05), 4,156 out of 26,046 islands analysed (16.0%) were differentially methylated, 4,138 (99.6%) of which showed hypermethylation after ischemia. These islands corresponded to 2,388 unique genes. Interestingly, the CpG island with the highest increase in methylation was located in the DDR1 promoter, a gene known to be involved in apoptosis and kidney fibrosis (FIG. 2D)¹¹.

TABLE 1
Donor, transplant and recipient characteristics of
the transplants included in the different cohorts.

| | Longitudinal | | Pre-implantation | | Post-reperfusion | |
|---|---|---|---|---|---|---|
| | cohort | | cohort | | cohort | |
| | (n = 13) | | (n = 82) | | (n = 46) | |
| Donor | | | | | | |
| Male/Female | 8/5 | | 43/39 | | 18/28 | |
| Age (y) | 43 ± 13 | | 47 ± 15 | | 50 ± 15 | |
| Last serum creatinine (mg/dL) | 0.81 ± 0.25 | | 0.74 ± 0.25 | | 0.71 ± 0.26 | (13 NA) |
| Expanded versus standard criteria | 1/12 | | 25/52 | (5 NA) | 17/26 | (3 NA) |
| donation | | | | | | |
| Transplant | | | | | | |
| Cold ischemia time (h) | 10.08 ± 4.15 | | 13.09 ± 3.96 | | 14.59 ± 4.68 | |
| Anastomosis time (min) | 32 ± 5 | | 36 ± 9 | | 33 ± 6 | |
| Recipient | | | | | | |
| Male/Female | 8/5 | | 54/28 | | 32/14 | |
| Age (y) | 55 ± 11 | | 55 ± 12 | | 57 ± 11 | |
| Number HLA mismatch | 3 ± 1 | | 3 ± 1 | (3 NA) | 2 ± 1 | (1 NA) |
| Post-Transplant | | | | | | |
| CADI score at 3 Mo | 3 ± 2 | (2 NA) | 3 ± 2 | (10 NA) | 2 ± 2 | (1 NA) |

| | | | | | |
|---|---|---|---|---|---|
| CADI score at 1 Year | 4 ± 2 | (5 NA) | 4 ± 2 | (23 NA) | 3 ± 2 |
| eGFR at 1 Year (ml/min/1.73 m²) | 45 ± 13 | (6 NA) | 52 ± 14 | (11 NA) | 50 ± 20 |

NA: no data available for this number of patients (n)

### Example 2. Loss of DNA Hydroxymethylation Upon Ischemia

Since it was recently demonstrated that low oxygen levels in tumors inhibit DNA demethylation by reducing TET activity⁸, and since in post-ischemic biopsies hypermethylation was enriched near CpG islands, which are preferential targets of TET enzymes⁷, we measured the product of TET activity, i.e. 5hmC. Specifically, we determined 5hmC levels genome-wide at >850,000 CpGs in six paired biopsies from our longitudinal cohort. Mean 5hmC levels were lower in post- versus pre-ischemia transplants (P<0.0001 for all transplants, FIG. 3A), indicating that ischemia reduces 5hmC levels in the kidney. We then evaluated locus-specifically whether changes in 5hmC are mirrored by inverse changes in 5mC. 5hmC was indeed decreased in 351,966 of the 427,724 (82.3%) CpGs whose 5mC levels increased following ischemia. When considering CpGs at P<0.05, both for the 5hmC and 5mC comparison, this relationship was even more striking: 1,353 of 1,354 (99.8%) of CpGs with a 5mC increase showed 5hmC loss (FIG. 3C). Reductions in 5hmC were not due to changes in TET expression as expression of TET1, TET2 and TET3 were unaltered in paired pre- versus post-ischemic biopsies (P>0.05). Likewise, expression of DNA methyltransferases, i.e., DNMT1, DNMT3A, DNMT3B and DNMT3L, was unchanged.

Finally, we confirmed the loss of 5hmC upon ischemia using liquid chromatography coupled to mass spectrometry (LC-MS) by comparing five post-reperfusion biopsies obtained from brain-dead donors characterized by long ischemia time (17.9±4.4 hours) versus five biopsies obtained from living donors undergoing minimal ischemia (32±6 minutes). Warm ischemia (anastomosis) times were comparable between both groups. 5hmC levels in kidney transplants from deceased donors were on average 16.4±4.4% lower compared to kidney transplants from living donors (P=0.006, FIG. 3B). Together, these findings suggest that upon ischemia kidney allografts become hypermethylated due to reduced TET activity.

### Example 3. Dose-Dependency of Ischemia-Induced DNA Methylation Changes

Each additional hour of cold ischemia time increases the risk of developing chronic allograft failure¹². Therefore, we assessed whether a similar correlation exists between cold ischemia time and the extent to which ischemia-induced methylation changes occur. We assembled a second independent cross-sectional cohort of 82 post-ischemic pre-implantation biopsies (Table 1, FIG. 1). In pre-implantation biopsies DNA methylation levels cannot be affected by warm ischemia nor reperfusion, and therefore cell composition changes cannot occur, excluding the possibility that changes in cell type composition underlie the methylation changes.

Cold ischemia time ranged from 4.7 to 26.7 hours. Genome-wide DNA methylation levels analysed using Illumina EPIC beadchips were correlated with cold ischemia time using a linear regression adjusted for donor gender and age. Methylation levels correlated with cold ischemia time for 29,700 CpG sites (P<0.05), the bulk of these (21,413 CpGs, 72.1%) showing ischemia-time dependent hypermethylation (P<0.00001, FIG. 4A). In some CpGs, methylation increased up to 2.6% with each hour increase in cold ischemia time. These CpGs were also more likely to be hypermethylated in the post-ischemic biopsies analysed in the longitudinal cohort (P<0.0001). Particularly, up to 2,932 CpGs were hypermethylated in both cohorts (P<0.05) and mainly affected CpG islands and shores, and less frequently shelves and open sea regions (FIG. 4B). When classifying these 2,932 CpGs based on kidney chromatin state, these CpGs were predominantly found at enhancers and gene promoters (FIG. 4C), which is in line with known TET-binding sites⁷.

At the CpG island level, cold ischemia time significantly correlated with methylation levels of 189 CpG islands (FDR<0.05, adjusted for age and gender). The vast majority of these were hypermethylated (156 islands, 82.5%, FIG. 4D). Of these 156 CpG islands, 66 (42.3%) were also hypermethylated at an FDR<0.05 threshold in the longitudinal cohort (versus 15.9% expected by random chance; P<0.00001, FIG. 4E; Table 2). We thus identified 66 CpG islands that were consistently hypermethylated at a stringent multiple correction threshold in both cohorts.

TABLE 2
Validated 66 CpG islands containing multiple hypermethylated CpGs.

| | | | longitudinal cohort | | | pre-implantation cohort | | | |
|---|---|---|---|---|---|---|---|---|---|
| | | | average % | | | % methylation | | | |
| | | | methylation | | | increase with | | | |
| | | | increase after | | | cold ischemia | | | |
| CpG island | n CpGs | location | ischemia | p value | FDR value | time (h) | p value | FDR value | CpG names |
| chr1: 152008838- | 21 | promoter | 0.91 | 0.00122584 | 0.014203 | 0.99 | 1.77E−05 | 0.009603725 | cg08210896, |
| 152009112 | | of | | | | | | | cg07853082, |
| | | S100A11 | | | | | | | cg01603146, |
| | | | | | | | | | cg07240554, |
| | | | | | | | | | cg12048339, |
| | | | | | | | | | cg03724763, |
| | | | | | | | | | cg06659614, |
| | | | | | | | | | cg03106313, |
| | | | | | | | | | cg11112162, |
| | | | | | | | | | cg12052258, |
| | | | | | | | | | cg25004833, |
| | | | | | | | | | cg12280317, |
| | | | | | | | | | cg11576590, |
| | | | | | | | | | cg27102304, |
| | | | | | | | | | cg06366009, |
| | | | | | | | | | cg06767701, |
| | | | | | | | | | cg12447069, |
| | | | | | | | | | cg26257241, |
| | | | | | | | | | cg10673431, |
| | | | | | | | | | cg19930352, |
| | | | | | | | | | cg10069121 |
| chr1: 156877769- | 11 | body of | 0.92 | 0.00534559 | 0.038574 | 0.87 | 0.00025 | 0.040838476 | cg00497172, |
| 156878649 | | PEAR1 | | | | | | | cg01796075, |
| | | | | | | | | | cg25761521, |
| | | | | | | | | | cg10252315, |
| | | | | | | | | | cg08685714, |
| | | | | | | | | | cg23731781, |
| | | | | | | | | | cg20792208, |
| | | | | | | | | | cg24995976, |
| | | | | | | | | | cg16090143, |
| | | | | | | | | | cg17967261, |
| | | | | | | | | | cg10871717 |
| chr1: 16085147- | 25 | promoter | 1.33 | 7.12E−05 | 0.00211 | 0.87 | 1.92E−05 | 0.009716415 | cg13484546, |
| 16085862 | | of | | | | | | | cg07107113, |
| | | FBLIM1 | | | | | | | cg25719573, |
| | | | | | | | | | cg15816719, |
| | | | | | | | | | cg03305795, |
| | | | | | | | | | cg04897742, |
| | | | | | | | | | cg16519300, |
| | | | | | | | | | cg16004427, |
| | | | | | | | | | cg17150168, |
| | | | | | | | | | cg17276021, |
| | | | | | | | | | cg23002761, |
| | | | | | | | | | cg08779336, |
| | | | | | | | | | cg11498041, |
| | | | | | | | | | cg11780735, |
| | | | | | | | | | cg01709096, |
| | | | | | | | | | cg14531560, |
| | | | | | | | | | cg07846167, |
| | | | | | | | | | cg02375669, |
| | | | | | | | | | cg01234724, |
| | | | | | | | | | cg26036626, |
| | | | | | | | | | cg22010340, |
| | | | | | | | | | cg14451275, |
| | | | | | | | | | cg25494767, |
| | | | | | | | | | cg15472071, |
| | | | | | | | | | cg04315300 |
| chr1: 19970255- | 34 | promoter | 1.29 | 6.02E−11 | 3.65E−08 | 0.59 | 8.66E−05 | 0.022781923 | cg00932104, |
| 19971923 | | of | | | | | | | cg03438613, |
| | | NBL1, | | | | | | | cg14304787, |
| | | promoter | | | | | | | cg03950225, |
| | | of | | | | | | | cg20890713, |
| | | MINOS1- | | | | | | | cg07147364, |
| | | NBL1 | | | | | | | cg16650717, |
| | | | | | | | | | cg21057046, |
| | | | | | | | | | cg18301528, |
| | | | | | | | | | cg22767145, |
| | | | | | | | | | cg21813747, |
| | | | | | | | | | cg09309058, |
| | | | | | | | | | cg12474394, |
| | | | | | | | | | cg07367302, |
| | | | | | | | | | cg14579430, |
| | | | | | | | | | cg15589641, |
| | | | | | | | | | cg17124647, |
| | | | | | | | | | cg25235465, |
| | | | | | | | | | cg10211745, |
| | | | | | | | | | cg10555159, |
| | | | | | | | | | cg10923719, |
| | | | | | | | | | cg03884082, |
| | | | | | | | | | cg19234140, |
| | | | | | | | | | cg18923740, |
| | | | | | | | | | cg20141851, |
| | | | | | | | | | cg08019633, |
| | | | | | | | | | cg14714346, |
| | | | | | | | | | cg15317859, |
| | | | | | | | | | cg09860653, |
| | | | | | | | | | cg04604347, |
| | | | | | | | | | cg05481086, |
| | | | | | | | | | cg18045201, |
| | | | | | | | | | cg14699309, |
| | | | | | | | | | cg19136075 |
| chr1: 32169537- | 19 | promoter | 1.42 | 6.03E−11 | 3.65E−08 | 0.75 | 9.86E−05 | 0.025175867 | cg05003322, |
| 32169869 | | of | | | | | | | cg23305408, |
| | | COL16A1 | | | | | | | cg00566320, |
| | | | | | | | | | cg13411999, |
| | | | | | | | | | cg02989257, |
| | | | | | | | | | cg22300839, |
| | | | | | | | | | cg27650656, |
| | | | | | | | | | cg00160583, |
| | | | | | | | | | cg09255732, |
| | | | | | | | | | cg21911647, |
| | | | | | | | | | cg16164167, |
| | | | | | | | | | cg09267996, |
| | | | | | | | | | cg27192620, |
| | | | | | | | | | cg19100596, |
| | | | | | | | | | cg24821709, |
| | | | | | | | | | cg04689698, |
| | | | | | | | | | cg16553500, |
| | | | | | | | | | cg13299148, |
| | | | | | | | | | cg04852949 |
| chr2: 27579296- | 18 | promoter | 0.30 | 0.00194542 | 0.019392 | 0.75 | 0.0002 | 0.036716868 | cg17509807, |
| 27580135 | | of | | | | | | | cg05106359, |
| | | GTF3C2 | | | | | | | cg19364957, |
| | | | | | | | | | cg23878109, |
| | | | | | | | | | cg01121072, |
| | | | | | | | | | cg07150314, |
| | | | | | | | | | cg23306799, |
| | | | | | | | | | cg16970492, |
| | | | | | | | | | cg25462815, |
| | | | | | | | | | cg10982590, |
| | | | | | | | | | cg16233353, |
| | | | | | | | | | cg12330118, |
| | | | | | | | | | cg11096970, |
| | | | | | | | | | cg25223497, |
| | | | | | | | | | cg17151420, |
| | | | | | | | | | cg07690667, |
| | | | | | | | | | cg22903655, |
| | | | | | | | | | cg25274833 |
| chr2: 66672431- | 21 | body of | 1.89 | 8.55E−15 | 2.47E−11 | 0.82 | 2.57E−06 | 0.002231103 | cg12082609, |
| 66673636 | | MEIS1 | | | | | | | cg02551743, |
| | | | | | | | | | cg21715346, |
| | | | | | | | | | cg08238215, |
| | | | | | | | | | cg06623961, |
| | | | | | | | | | cg01271812, |
| | | | | | | | | | cg14775296, |
| | | | | | | | | | cg06833110, |
| | | | | | | | | | cg04751149, |
| | | | | | | | | | cg09535924, |
| | | | | | | | | | cg13169081, |
| | | | | | | | | | cg03175652, |
| | | | | | | | | | cg06880482, |
| | | | | | | | | | cg11202254, |
| | | | | | | | | | cg10464312, |
| | | | | | | | | | cg06994420, |
| | | | | | | | | | cg02492115, |
| | | | | | | | | | cg11357542, |
| | | | | | | | | | cg15468045, |
| | | | | | | | | | cg09550083, |
| | | | | | | | | | cg12407996 |
| chr2: 74781494- | 26 | promoter | 0.54 | 0.00626869 | 0.042868 | 0.6 | 0.00016 | 0.032221072 | cg13004765, |
| 74782685 | | and | | | | | | | cg13690241, |
| | | body of | | | | | | | cg14012686, |
| | | DOK1, | | | | | | | cg23674882, |
| | | promoter | | | | | | | cg25142466, |
| | | of | | | | | | | cg22238923, |
| | | LOXL3, | | | | | | | cg02048416, |
| | | 3′UTR of | | | | | | | cg20706438, |
| | | C2orf65 | | | | | | | cg11800635, |
| | | | | | | | | | cg22989958, |
| | | | | | | | | | cg25792271, |
| | | | | | | | | | cg26117023, |
| | | | | | | | | | cg12962355, |
| | | | | | | | | | cg13706325, |
| | | | | | | | | | cg08940570, |
| | | | | | | | | | cg23604158, |
| | | | | | | | | | cg17891101, |
| | | | | | | | | | cg00831247, |
| | | | | | | | | | cg11473080, |
| | | | | | | | | | cg13694405, |
| | | | | | | | | | cg25063221, |
| | | | | | | | | | cg03101068, |
| | | | | | | | | | cg15989091, |
| | | | | | | | | | cg20288165, |
| | | | | | | | | | cg19148731, |
| | | | | | | | | | cg11067786 |
| chr2: 85640969- | 25 | promoter | 1.29 | 0.00012104 | 0.00311 | 1.14 | 1.89E−05 | 0.009716415 | cg09337254, |
| 85641259 | | of | | | | | | | cg01948944, |
| | | CAPG | | | | | | | cg27383365, |
| | | | | | | | | | cg03718845, |
| | | | | | | | | | cg21647532, |
| | | | | | | | | | cg14825368, |
| | | | | | | | | | cg16794227, |
| | | | | | | | | | cg27139457, |
| | | | | | | | | | cg17201966, |
| | | | | | | | | | cg19627006, |
| | | | | | | | | | cg26476820, |
| | | | | | | | | | cg14239629, |
| | | | | | | | | | cg13217878, |
| | | | | | | | | | cg10664272, |
| | | | | | | | | | cg20207544, |
| | | | | | | | | | cg02242344, |
| | | | | | | | | | cg18845187, |
| | | | | | | | | | cg25358315, |
| | | | | | | | | | cg16437908, |
| | | | | | | | | | cg16838838, |
| | | | | | | | | | cg12225712, |
| | | | | | | | | | cg07215695, |
| | | | | | | | | | cg25161092, |
| | | | | | | | | | Cg23189291, |
| | | | | | | | | | cg21654383 |
| chr2: 85980499- | 23 | promoter | 0.50 | 0.00165714 | 0.017397 | 0.86 | 2.46E−05 | 0.011240042 | cg07381326, |
| 85982198 | | and | | | | | | | cg19956166, |
| | | body of | | | | | | | cg05779007, |
| | | ATOH8 | | | | | | | cg02317742, |
| | | | | | | | | | cg15649452, |
| | | | | | | | | | cg03128635, |
| | | | | | | | | | cg17225651, |
| | | | | | | | | | cg13841286, |
| | | | | | | | | | cg14558812, |
| | | | | | | | | | cg07272719, |
| | | | | | | | | | cg00400334, |
| | | | | | | | | | cg01461067, |
| | | | | | | | | | cg09662694, |
| | | | | | | | | | cg02696047, |
| | | | | | | | | | cg12930553, |
| | | | | | | | | | cg13065834, |
| | | | | | | | | | cg06897686, |
| | | | | | | | | | cg01751470, |
| | | | | | | | | | cg21068480, |
| | | | | | | | | | cg06285619, |
| | | | | | | | | | cg15671782, |
| | | | | | | | | | cg18815025, |
| | | | | | | | | | cg24399924 |
| chr3: 128205495- | 44 | promoter | 0.66 | 2.92E−05 | 0.001127 | 0.54 | 3.50E−05 | 0.013468469 | cg22122410, |
| 128212274 | | and | | | | | | | cg25395660, |
| | | body of | | | | | | | cg16674492, |
| | | GATA2 | | | | | | | cg06490988, |
| | | | | | | | | | cg02436004, |
| | | | | | | | | | cg13442299, |
| | | | | | | | | | cg21675036, |
| | | | | | | | | | cg19759549, |
| | | | | | | | | | cg07841173, |
| | | | | | | | | | cg24334648, |
| | | | | | | | | | cg19638477, |
| | | | | | | | | | cg27106398, |
| | | | | | | | | | cg09852607, |
| | | | | | | | | | cg12356743, |
| | | | | | | | | | cg02856377, |
| | | | | | | | | | cg22931738, |
| | | | | | | | | | cg21435190, |
| | | | | | | | | | cg02980693, |
| | | | | | | | | | cg07132710, |
| | | | | | | | | | cg22801992, |
| | | | | | | | | | cg04347582, |
| | | | | | | | | | cg02836487, |
| | | | | | | | | | cg06796779, |
| | | | | | | | | | cg03839949, |
| | | | | | | | | | cg18065337, |
| | | | | | | | | | cg21294440, |
| | | | | | | | | | cg10935762, |
| | | | | | | | | | cg06115614, |
| | | | | | | | | | cg21712811, |
| | | | | | | | | | cg13483882, |
| | | | | | | | | | cg22915582, |
| | | | | | | | | | cg09024124, |
| | | | | | | | | | cg19301963, |
| | | | | | | | | | cg25686860, |
| | | | | | | | | | cg23335389, |
| | | | | | | | | | cg01102073, |
| | | | | | | | | | cg23520930, |
| | | | | | | | | | cg07195926, |
| | | | | | | | | | cg00847029, |
| | | | | | | | | | cg13808674, |
| | | | | | | | | | cg08755743, |
| | | | | | | | | | cg17642618, |
| | | | | | | | | | cg07263393, |
| | | | | | | | | | cg25229470 |
| chr3: 146187108- | 10 | promoter | 1.73 | 3.93E−05 | 0.001396 | 2.22 | 3.38E−07 | 0.00055018 | cg19917720, |
| 146187710 | | and | | | | | | | cg20069430, |
| | | body of | | | | | | | cg21569635, |
| | | PLSCR2 | | | | | | | cg26794949, |
| | | | | | | | | | cg24092307, |
| | | | | | | | | | cg24607783, |
| | | | | | | | | | cg24005645, |
| | | | | | | | | | cg02128651, |
| | | | | | | | | | cg14056864, |
| | | | | | | | | | cg04442406 |
| chr3: 170136242- | 21 | promoter | 1.03 | 4.65E−09 | 1.35E−06 | 0.83 | 0.00013 | 0.028633263 | cg20794824, |
| 170137886 | | and | | | | | | | cg07126617, |
| | | body of | | | | | | | cg12741994, |
| | | CLDN11 | | | | | | | cg09281405, |
| | | | | | | | | | cg01591313, |
| | | | | | | | | | cg02241055, |
| | | | | | | | | | cg03916832, |
| | | | | | | | | | cg17078427, |
| | | | | | | | | | cg00894757, |
| | | | | | | | | | cg20924286, |
| | | | | | | | | | cg06023994, |
| | | | | | | | | | cg20449692, |
| | | | | | | | | | cg07042832, |
| | | | | | | | | | cg11145160, |
| | | | | | | | | | cg09389280, |
| | | | | | | | | | cg23965165, |
| | | | | | | | | | cg07434518, |
| | | | | | | | | | cg11609327, |
| | | | | | | | | | cg13333304, |
| | | | | | | | | | cg13879776, |
| | | | | | | | | | cg07137845 |
| chr3: 44802852- | 18 | promoter | 0.80 | 2.64E−05 | 0.001056 | 1.39 | 7.74E−06 | 0.00559946 | cg17372269, |
| 44803618 | | and | | | | | | | cg22314314, |
| | | body of | | | | | | | cg15225532, |
| | | KIF15, | | | | | | | cg26151597, |
| | | promoter | | | | | | | cg09333631, |
| | | and | | | | | | | cg24858591, |
| | | body of | | | | | | | cg10348013, |
| | | KIAA1143 | | | | | | | cg09053247, |
| | | | | | | | | | cg19759251, |
| | | | | | | | | | cg00702638, |
| | | | | | | | | | cg10337772, |
| | | | | | | | | | cg14965968, |
| | | | | | | | | | cg24888989, |
| | | | | | | | | | cg19349877, |
| | | | | | | | | | cg22954484, |
| | | | | | | | | | cg17546649, |
| | | | | | | | | | cg07801283, |
| | | | | | | | | | cg18086594 |
| chr4: 4864456- | 18 | body of | 0.70 | 0.00120177 | 0.014012 | 0.66 | 0.0003 | 0.045570045 | cg06375949, |
| 4864834 | | MSX1 | | | | | | | cg15848031, |
| | | | | | | | | | cg01785568, |
| | | | | | | | | | cg21538208, |
| | | | | | | | | | cg09573795, |
| | | | | | | | | | cg14769943, |
| | | | | | | | | | cg20161179, |
| | | | | | | | | | cg03199651, |
| | | | | | | | | | cg25144207, |
| | | | | | | | | | cg03843978, |
| | | | | | | | | | cg27597123, |
| | | | | | | | | | cg22609784, |
| | | | | | | | | | cg24840099, |
| | | | | | | | | | cg09748975, |
| | | | | | | | | | cg20891301, |
| | | | | | | | | | cg14167596, |
| | | | | | | | | | cg19596468, |
| | | | | | | | | | cg27038439 |
| chr4: 79472806- | 14 | promoter | 1.26 | 0.00703026 | 0.04624 | 0.94 | 0.00019 | 0.035626493 | cg01807770, |
| 79473177 | | of | | | | | | | cg19965948, |
| | | ANXA3 | | | | | | | cg06964816, |
| | | | | | | | | | cg09581228, |
| | | | | | | | | | cg26656300, |
| | | | | | | | | | cg20456136, |
| | | | | | | | | | cg18787914, |
| | | | | | | | | | cg08908264, |
| | | | | | | | | | cg01473247, |
| | | | | | | | | | cg10616442, |
| | | | | | | | | | cg19069553, |
| | | | | | | | | | cg00319655, |
| | | | | | | | | | cg19631365, |
| | | | | | | | | | cg12225685 |
| chr5: 150051116- | 17 | body of | 0.90 | 0.00286004 | 0.025269 | 1.14 | 0.0003 | 0.045570045 | cg15699693, |
| 150052107 | | MYOZ3 | | | | | | | cg07283463, |
| | | | | | | | | | cg11590420, |
| | | | | | | | | | cg22538396, |
| | | | | | | | | | cg01900559, |
| | | | | | | | | | cg24157272, |
| | | | | | | | | | cg15674825, |
| | | | | | | | | | cg14449863, |
| | | | | | | | | | cg15675367, |
| | | | | | | | | | cg14771810, |
| | | | | | | | | | cg25665736, |
| | | | | | | | | | cg03901247, |
| | | | | | | | | | cg23787867, |
| | | | | | | | | | cg26784201, |
| | | | | | | | | | cg09187633, |
| | | | | | | | | | cg04430244, |
| | | | | | | | | | cg14111464 |
| chr6: 10882926- | 14 | promoter | 0.62 | 0.00221371 | 0.02112 | 0.93 | 1.73E−05 | 0.009586409 | cg24113409, |
| 10883149 | | of | | | | | | | cg13726504, |
| | | GCM2 | | | | | | | cg10074727, |
| | | | | | | | | | cg06085647, |
| | | | | | | | | | cg20180247, |
| | | | | | | | | | cg17991695, |
| | | | | | | | | | cg09829319, |
| | | | | | | | | | cg09775263, |
| | | | | | | | | | cg14176930, |
| | | | | | | | | | cg19951298, |
| | | | | | | | | | cg03017829, |
| | | | | | | | | | cg08510658, |
| | | | | | | | | | cg24329557, |
| | | | | | | | | | cg14250833 |
| chr6: 30852102- | 64 | promoter | 1.79 | 1.53E−28 | 3.99E−24 | 0.96 | 1.63E−11 | 1.06E−07 | cg09281154, |
| 30852676 | | of | | | | | | | cg06012011, |
| | | DDR1 | | | | | | | cg25075776, |
| | | | | | | | | | cg11977634, |
| | | | | | | | | | cg11676038, |
| | | | | | | | | | cg23953820, |
| | | | | | | | | | cg26556926, |
| | | | | | | | | | cg08684361, |
| | | | | | | | | | cg24303888, |
| | | | | | | | | | cg00204743, |
| | | | | | | | | | cg25251478, |
| | | | | | | | | | cg07939626, |
| | | | | | | | | | cg00536341, |
| | | | | | | | | | cg21249595, |
| | | | | | | | | | cg19894264, |
| | | | | | | | | | cg16079541, |
| | | | | | | | | | cg26858073, |
| | | | | | | | | | cg11975790, |
| | | | | | | | | | cg16215084, |
| | | | | | | | | | cg15516187, |
| | | | | | | | | | cg08469255, |
| | | | | | | | | | cg12847793, |
| | | | | | | | | | cg13660719, |
| | | | | | | | | | cg13695585, |
| | | | | | | | | | cg18093866, |
| | | | | | | | | | cg06642647, |
| | | | | | | | | | cg07265873, |
| | | | | | | | | | cg20955507, |
| | | | | | | | | | cg24646556, |
| | | | | | | | | | cg23001000, |
| | | | | | | | | | cg00466425, |
| | | | | | | | | | cg13329862, |
| | | | | | | | | | cg19215110, |
| | | | | | | | | | cg05703744, |
| | | | | | | | | | cg14279856, |
| | | | | | | | | | cg02695062, |
| | | | | | | | | | cg07803420, |
| | | | | | | | | | cg16537676, |
| | | | | | | | | | cg06893977, |
| | | | | | | | | | cg12308216, |
| | | | | | | | | | cg11530564, |
| | | | | | | | | | cg22485298, |
| | | | | | | | | | cg25607383, |
| | | | | | | | | | cg07908039, |
| | | | | | | | | | cg24727290, |
| | | | | | | | | | cg26321999, |
| | | | | | | | | | cg02696067, |
| | | | | | | | | | cg03270204, |
| | | | | | | | | | cg16797094, |
| | | | | | | | | | cg09822812, |
| | | | | | | | | | cg00934322, |
| | | | | | | | | | cg19018599, |
| | | | | | | | | | cg15656686, |
| | | | | | | | | | cg07187855, |
| | | | | | | | | | cg17091577, |
| | | | | | | | | | cg09965419, |
| | | | | | | | | | cg19591099, |
| | | | | | | | | | cg13396738, |
| | | | | | | | | | cg24566261, |
| | | | | | | | | | cg25655106, |
| | | | | | | | | | cg13351860, |
| | | | | | | | | | cg17604312, |
| | | | | | | | | | cg08951271, |
| | | | | | | | | | cg06501109 |
| chr6: 32121829- | 81 | promoter | 1.17 | 5.75E−15 | 2.14E−11 | 0.6 | 4.15E−07 | 0.000568856 | cg19048176, |
| 32122529 | | and | | | | | | | cg04749507, |
| | | body of | | | | | | | cg09599399, |
| | | PPT2, | | | | | | | cg17784596, |
| | | promoter | | | | | | | cg06814287, |
| | | of | | | | | | | cg17161421, |
| | | PRRT1 | | | | | | | cg18235088, |
| | | | | | | | | | cg08045906, |
| | | | | | | | | | cg05133205, |
| | | | | | | | | | cg08509237, |
| | | | | | | | | | cg21037008, |
| | | | | | | | | | cg26567592, |
| | | | | | | | | | cg04264374, |
| | | | | | | | | | cg10369585, |
| | | | | | | | | | cg17329164, |
| | | | | | | | | | cg11229390, |
| | | | | | | | | | cg17513693, |
| | | | | | | | | | cg12883279, |
| | | | | | | | | | cg06264679, |
| | | | | | | | | | cg00403498, |
| | | | | | | | | | cg05465342, |
| | | | | | | | | | cg08110052, |
| | | | | | | | | | cg02429905, |
| | | | | | | | | | cg02956248, |
| | | | | | | | | | cg12568595, |
| | | | | | | | | | cg16101080, |
| | | | | | | | | | cg17383811, |
| | | | | | | | | | cg03784567, |
| | | | | | | | | | cg00933538, |
| | | | | | | | | | cg23470939, |
| | | | | | | | | | cg09672152, |
| | | | | | | | | | cg20914572, |
| | | | | | | | | | cg13934406, |
| | | | | | | | | | cg27134827, |
| | | | | | | | | | cg11192767, |
| | | | | | | | | | cg27631107, |
| | | | | | | | | | cg17113856, |
| | | | | | | | | | cg03010186, |
| | | | | | | | | | cg18049167, |
| | | | | | | | | | cg20981412, |
| | | | | | | | | | cg20771808, |
| | | | | | | | | | cg02460426, |
| | | | | | | | | | cg12626589, |
| | | | | | | | | | cg25426302, |
| | | | | | | | | | cg06025456, |
| | | | | | | | | | cg17229678, |
| | | | | | | | | | cg08057899, |
| | | | | | | | | | cg04528217, |
| | | | | | | | | | cg11386011, |
| | | | | | | | | | cg04194294, |
| | | | | | | | | | cg14130039, |
| | | | | | | | | | cg24283914, |
| | | | | | | | | | cg04105091, |
| | | | | | | | | | cg12585943, |
| | | | | | | | | | cg00552704, |
| | | | | | | | | | cg04877280, |
| | | | | | | | | | cg09714607, |
| | | | | | | | | | cg11941520, |
| | | | | | | | | | cg26169408, |
| | | | | | | | | | cg11122280, |
| | | | | | | | | | cg23660356, |
| | | | | | | | | | cg03570994, |
| | | | | | | | | | cg12738718, |
| | | | | | | | | | cg13102294, |
| | | | | | | | | | cg06108383, |
| | | | | | | | | | cg27070869, |
| | | | | | | | | | cg08814206, |
| | | | | | | | | | cg04856022, |
| | | | | | | | | | cg06902929, |
| | | | | | | | | | cg24509300, |
| | | | | | | | | | cg03434432, |
| | | | | | | | | | cg03995156, |
| | | | | | | | | | cg00086577, |
| | | | | | | | | | cg00110832, |
| | | | | | | | | | cg23359665, |
| | | | | | | | | | cg20328456, |
| | | | | | | | | | cg10551329, |
| | | | | | | | | | cg21241317, |
| | | | | | | | | | cg04536704, |
| | | | | | | | | | cg16481280, |
| | | | | | | | | | cg01111041 |
| chr6: 33244677- | 71 | promoter | 1.26 | 1.13E−11 | 1.05E−08 | 0.97 | 1.05E−06 | 0.001093848 | cg11772919, |
| 33245554 | | of | | | | | | | cg17416730, |
| | | B3GALT4 | | | | | | | cg16428857, |
| | | | | | | | | | cg07348922, |
| | | | | | | | | | cg16396284, |
| | | | | | | | | | cg19241689, |
| | | | | | | | | | cg08306084, |
| | | | | | | | | | cg04262471, |
| | | | | | | | | | cg03127244, |
| | | | | | | | | | cg21699833, |
| | | | | | | | | | cg26270195, |
| | | | | | | | | | cg21333861, |
| | | | | | | | | | cg26381352, |
| | | | | | | | | | cg19268452, |
| | | | | | | | | | cg06753439, |
| | | | | | | | | | cg12395726, |
| | | | | | | | | | cg14069465, |
| | | | | | | | | | cg15543281, |
| | | | | | | | | | cg01807737, |
| | | | | | | | | | cg03189210, |
| | | | | | | | | | cg18932158, |
| | | | | | | | | | cg11400761, |
| | | | | | | | | | cg13365340, |
| | | | | | | | | | cg00052772, |
| | | | | | | | | | cg17453433, |
| | | | | | | | | | cg27098900, |
| | | | | | | | | | cg27373972, |
| | | | | | | | | | cg11129609, |
| | | | | | | | | | cg19271658, |
| | | | | | | | | | cg10633838, |
| | | | | | | | | | cg10980449, |
| | | | | | | | | | cg04263436, |
| | | | | | | | | | cg21618521, |
| | | | | | | | | | cg08483834, |
| | | | | | | | | | cg19882268, |
| | | | | | | | | | cg19156220, |
| | | | | | | | | | cg21387418, |
| | | | | | | | | | cg09730719, |
| | | | | | | | | | cg27147350, |
| | | | | | | | | | cg19664267, |
| | | | | | | | | | cg21334198, |
| | | | | | | | | | cg22878489, |
| | | | | | | | | | cg03721978, |
| | | | | | | | | | cg11626629, |
| | | | | | | | | | cg23950233, |
| | | | | | | | | | cg02299465, |
| | | | | | | | | | cg03702686, |
| | | | | | | | | | cg22322679, |
| | | | | | | | | | cg08085929, |
| | | | | | | | | | cg18729787, |
| | | | | | | | | | cg07306737, |
| | | | | | | | | | cg24605046, |
| | | | | | | | | | cg03833499, |
| | | | | | | | | | cg10111290, |
| | | | | | | | | | cg08090835, |
| | | | | | | | | | cg14023774, |
| | | | | | | | | | cg17103217, |
| | | | | | | | | | cg12583553, |
| | | | | | | | | | cg21986677, |
| | | | | | | | | | cg19873719, |
| | | | | | | | | | cg00163549, |
| | | | | | | | | | cg26912426, |
| | | | | | | | | | cg21859603, |
| | | | | | | | | | cg07556599, |
| | | | | | | | | | cg13882090, |
| | | | | | | | | | cg10426422, |
| | | | | | | | | | cg26055446, |
| | | | | | | | | | cg16580935, |
| | | | | | | | | | cg16090881, |
| | | | | | | | | | cg16226644, |
| | | | | | | | | | cg09080120 |
| chr6: 37503538- | 15 | ?, body | 1.57 | 3.54E−05 | 0.001295 | 2.59 | 4.20E−11 | 2.19E−07 | cg25019722, |
| 37504291 | | of | | | | | | | cg16150900, |
| | | LOC100505530 | | | | | | | cg01843034, |
| | | | | | | | | | cg21415424, |
| | | | | | | | | | cg21545147, |
| | | | | | | | | | cg24807547, |
| | | | | | | | | | cg26579986, |
| | | | | | | | | | cg00423647, |
| | | | | | | | | | cg08126542, |
| | | | | | | | | | cg00360474, |
| | | | | | | | | | cg00340231, |
| | | | | | | | | | cg18877699, |
| | | | | | | | | | cg16726195, |
| | | | | | | | | | cg18465199, |
| | | | | | | | | | cg11522683 |
| chr6: 44187186- | 18 | promoter | 0.93 | 5.93E−06 | 0.000326 | 0.8091776 | 0.00012 | 0.027156853 | cg07252933, |
| 44187400 | | of | | | | | | | cg00330501, |
| | | SLC29A1 | | | | | | | cg04175292, |
| | | | | | | | | | cg17217665, |
| | | | | | | | | | cg04742345, |
| | | | | | | | | | cg27078824, |
| | | | | | | | | | cg07561710, |
| | | | | | | | | | cg27593649, |
| | | | | | | | | | cg23737112, |
| | | | | | | | | | cg01993576, |
| | | | | | | | | | cg11452354, |
| | | | | | | | | | cg21636621, |
| | | | | | | | | | cg10519140, |
| | | | | | | | | | cg07053014, |
| | | | | | | | | | cg22461515, |
| | | | | | | | | | cg03634967, |
| | | | | | | | | | cg13793145, |
| | | | | | | | | | cg06638377 |
| chr6: 56818873- | 16 | promoter | 0.40 | 0.00666901 | 0.04463 | 1.0201249 | 8.48E−06 | 0.005849883 | cg15140191, |
| 56820308 | | of | | | | | | | cg09270675, |
| | | BEND6, | | | | | | | cg21442906, |
| | | promoter | | | | | | | cg20459712, |
| | | of DST | | | | | | | cg17346177, |
| | | | | | | | | | cg04787343, |
| | | | | | | | | | cg09970511, |
| | | | | | | | | | cg27378522, |
| | | | | | | | | | cg01626459, |
| | | | | | | | | | cg02339682, |
| | | | | | | | | | cg11014463, |
| | | | | | | | | | cg01696193, |
| | | | | | | | | | cg22880620, |
| | | | | | | | | | cg05871997, |
| | | | | | | | | | cg26366048, |
| | | | | | | | | | cg24311272 |
| chr7: 120969587- | 18 | promoter | 0.71 | 0.0011074 | 0.013341 | 0.7142528 | 0.00018 | 0.035284567 | cg18579879, |
| 120970743 | | and | | | | | | | cg14448169, |
| | | body of | | | | | | | cg01311674, |
| | | WNT16 | | | | | | | cg04760021, |
| | | | | | | | | | cg14722104, |
| | | | | | | | | | cg01725608, |
| | | | | | | | | | cg25608490, |
| | | | | | | | | | cg26673903, |
| | | | | | | | | | cg03721528, |
| | | | | | | | | | cg12073479, |
| | | | | | | | | | cg09857513, |
| | | | | | | | | | cg05470554, |
| | | | | | | | | | cg19617672, |
| | | | | | | | | | cg26690075, |
| | | | | | | | | | cg13161961, |
| | | | | | | | | | cg00915831, |
| | | | | | | | | | cg16868298, |
| | | | | | | | | | cg26292912 |
| chr7: 27190274- | 24 | promoter | 1.06 | 4.54E−05 | 0.001554 | 1.0070883 | 6.27E−08 | 0.00013608 | cg06206902, |
| 27191115 | | of | | | | | | | cg16771406, |
| | | HOXA6, | | | | | | | cg06685968, |
| | | body of | | | | | | | cg04639396, |
| | | HOXA- | | | | | | | cg03547218, |
| | | AS3 | | | | | | | cg19816811, |
| | | | | | | | | | cg10739556, |
| | | | | | | | | | cg16880946, |
| | | | | | | | | | cg01210554, |
| | | | | | | | | | cg17969084, |
| | | | | | | | | | cg26032198, |
| | | | | | | | | | cg24398479, |
| | | | | | | | | | cg18690769, |
| | | | | | | | | | cg14109662, |
| | | | | | | | | | cg19623360, |
| | | | | | | | | | cg01414882, |
| | | | | | | | | | cg04073257, |
| | | | | | | | | | cg02919960, |
| | | | | | | | | | cg19943010, |
| | | | | | | | | | cg10374314, |
| | | | | | | | | | cg07807562, |
| | | | | | | | | | cg10343278, |
| | | | | | | | | | cg18344212, |
| | | | | | | | | | cg23590202 |
| chr7: 63505977- | 8 | promoter | 2.18 | 3.01E−06 | 0.000195 | 2.3619374 | 0.00011 | 0.026372154 | cg24975986, |
| 63506298 | | of | | | | | | | cg19155391, |
| | | ZNF727 | | | | | | | cg01176516, |
| | | | | | | | | | cg15473066, |
| | | | | | | | | | cg15949805, |
| | | | | | | | | | cg21783223, |
| | | | | | | | | | cg01849085, |
| | | | | | | | | | cg01760756 |
| chr8: 41165852- | 29 | promoter | 0.72 | 0.00178539 | 0.018378 | 0.5925679 | 0.00022 | 0.039063184 | cg01495122, |
| 41167140 | | of | | | | | | | cg01074584, |
| | | SFRP1 | | | | | | | cg14904908, |
| | | | | | | | | | cg03133371, |
| | | | | | | | | | cg04255616, |
| | | | | | | | | | cg14824386, |
| | | | | | | | | | cg07935886, |
| | | | | | | | | | cg13398291, |
| | | | | | | | | | cg03575666, |
| | | | | | | | | | cg09410389, |
| | | | | | | | | | cg00930833, |
| | | | | | | | | | cg21517947, |
| | | | | | | | | | cg10406295, |
| | | | | | | | | | cg14556146, |
| | | | | | | | | | cg06777844, |
| | | | | | | | | | cg21415450, |
| | | | | | | | | | cg00000321, |
| | | | | | | | | | cg06166767, |
| | | | | | | | | | cg14548509, |
| | | | | | | | | | cg02388150, |
| | | | | | | | | | cg24067169, |
| | | | | | | | | | cg15839448, |
| | | | | | | | | | cg22418909, |
| | | | | | | | | | cg24319902, |
| | | | | | | | | | cg23359714, |
| | | | | | | | | | cg16196274, |
| | | | | | | | | | cg05882344, |
| | | | | | | | | | cg01433296, |
| | | | | | | | | | cg16662821 |
| chr9: 1050078- | 16 | promoter | 0.75 | 7.80E−05 | 0.00226 | 0.806199 | 0.00026 | 0.042356651 | cg12273142, |
| 1050510 | | and | | | | | | | cg11242992, |
| | | body of | | | | | | | cg27657187, |
| | | DMRT2, | | | | | | | cg10787698, |
| | | body of | | | | | | | cg13863701, |
| | | LINC01230 | | | | | | | cg11795022, |
| | | | | | | | | | cg21080263, |
| | | | | | | | | | cg09315839, |
| | | | | | | | | | cg02991759, |
| | | | | | | | | | cg00934355, |
| | | | | | | | | | cg01803297, |
| | | | | | | | | | cg19464563, |
| | | | | | | | | | cg06495009, |
| | | | | | | | | | cg26133523, |
| | | | | | | | | | cg14036347, |
| | | | | | | | | | cg09934216 |
| chr10: 116163391- | 19 | promoter | 1.04 | 0.00553637 | 0.039442 | 0.8192897 | 3.43E−05 | 0.013468469 | cg20663200, |
| 116164599 | | and | | | | | | | cg01316152, |
| | | body of | | | | | | | cg04070533, |
| | | AFAP1L2 | | | | | | | cg20048434, |
| | | | | | | | | | cg26017408, |
| | | | | | | | | | cg20283670, |
| | | | | | | | | | cg13829736, |
| | | | | | | | | | cg19264606, |
| | | | | | | | | | cg20196291, |
| | | | | | | | | | cg15657704, |
| | | | | | | | | | cg13825376, |
| | | | | | | | | | cg11453400, |
| | | | | | | | | | cg00632403, |
| | | | | | | | | | cg19615406, |
| | | | | | | | | | cg00739593, |
| | | | | | | | | | cg22128849, |
| | | | | | | | | | cg01720316, |
| | | | | | | | | | cg06346505, |
| | | | | | | | | | cg10753764 |
| chr10: 8091374- | 65 | promoter | 0.46 | 2.82E−07 | 3.53E−05 | 0.5430113 | 1.94E−05 | 0.009716415 | cg13814485, |
| 8098329 | | and | | | | | | | cg04982951, |
| | | body of | | | | | | | cg04729913, |
| | | GATA3, | | | | | | | cg06022942, |
| | | promoter | | | | | | | cg20314737, |
| | | and | | | | | | | cg15852223, |
| | | body of | | | | | | | cg13543854, |
| | | FLJ45983 | | | | | | | cg08347183, |
| | | | | | | | | | cg24039697, |
| | | | | | | | | | cg03672342, |
| | | | | | | | | | cg06230736, |
| | | | | | | | | | cg22783180, |
| | | | | | | | | | cg19679989, |
| | | | | | | | | | cg17891011, |
| | | | | | | | | | cg11444332, |
| | | | | | | | | | cg11018337, |
| | | | | | | | | | cg12730771, |
| | | | | | | | | | cg27542609, |
| | | | | | | | | | cg25954627, |
| | | | | | | | | | cg23074048, |
| | | | | | | | | | cg17611674, |
| | | | | | | | | | cg00296182, |
| | | | | | | | | | cg23058185, |
| | | | | | | | | | cg15803869, |
| | | | | | | | | | cg11731114, |
| | | | | | | | | | cg06870728, |
| | | | | | | | | | cg15267232, |
| | | | | | | | | | cg19315863, |
| | | | | | | | | | cg05671070, |
| | | | | | | | | | cg15187550, |
| | | | | | | | | | cg25536137, |
| | | | | | | | | | cg20281962, |
| | | | | | | | | | cg11100386, |
| | | | | | | | | | cg15330117, |
| | | | | | | | | | cg18187680, |
| | | | | | | | | | cg07578663, |
| | | | | | | | | | cg23768829, |
| | | | | | | | | | cg26292521, |
| | | | | | | | | | cg13431023, |
| | | | | | | | | | cg16710894, |
| | | | | | | | | | cg04850366, |
| | | | | | | | | | cg25735492, |
| | | | | | | | | | cg12181459, |
| | | | | | | | | | cg24797840, |
| | | | | | | | | | cg17124583, |
| | | | | | | | | | cg23943136, |
| | | | | | | | | | cg22647713, |
| | | | | | | | | | cg17566118, |
| | | | | | | | | | cg09728012, |
| | | | | | | | | | cg01364137, |
| | | | | | | | | | cg24647276, |
| | | | | | | | | | cg04641787, |
| | | | | | | | | | cg05721515, |
| | | | | | | | | | cg04050331, |
| | | | | | | | | | cg07508910, |
| | | | | | | | | | cg19657198, |
| | | | | | | | | | cg01224891, |
| | | | | | | | | | cg04765277, |
| | | | | | | | | | cg08707112, |
| | | | | | | | | | cg05356738, |
| | | | | | | | | | cg07516470, |
| | | | | | | | | | cg00779924, |
| | | | | | | | | | cg14327531, |
| | | | | | | | | | cg14098681, |
| | | | | | | | | | cg18599069 |
| chr11: 119186947- | 20 | promoter | 0.64 | 0.00237302 | 0.022224 | 0.656425 | 0.00019 | 0.035573633 | cg04470256, |
| 119187894 | | and | | | | | | | cg11287851, |
| | | body of | | | | | | | cg24632644, |
| | | MCAM, | | | | | | | cg06273010, |
| | | promoter | | | | | | | cg26864130, |
| | | of | | | | | | | cg06338928, |
| | | MIR6756 | | | | | | | cg25161838, |
| | | | | | | | | | cg23230629, |
| | | | | | | | | | cg21096399, |
| | | | | | | | | | cg11906947, |
| | | | | | | | | | cg09042577, |
| | | | | | | | | | cg19491895, |
| | | | | | | | | | cg18165196, |
| | | | | | | | | | cg04890495, |
| | | | | | | | | | cg03365354, |
| | | | | | | | | | cg25484790, |
| | | | | | | | | | cg03558921, |
| | | | | | | | | | cg03545206, |
| | | | | | | | | | cg17622922, |
| | | | | | | | | | cg15050201 |
| chr11: 65325081- | 16 | promoter | 0.60 | 0.00070176 | 0.009896 | 1.1010865 | 4.75E−05 | 0.01507488 | cg02589497, |
| 65326209 | | of | | | | | | | cg23420791, |
| | | LTBP3 | | | | | | | cg14749448, |
| | | | | | | | | | cg14914204, |
| | | | | | | | | | cg16477774, |
| | | | | | | | | | cg02809401, |
| | | | | | | | | | cg08965235, |
| | | | | | | | | | cg11171811, |
| | | | | | | | | | cg16632280, |
| | | | | | | | | | cg04641114, |
| | | | | | | | | | cg05340623, |
| | | | | | | | | | cg17880403, |
| | | | | | | | | | cg14240304, |
| | | | | | | | | | cg12874602, |
| | | | | | | | | | cg22214565, |
| | | | | | | | | | cg17451760 |
| chr11: 79148358- | 30 | promoter | 0.49 | 0.00012542 | 0.003196 | 0.9617349 | 4.62E−05 | 0.01504041 | cg11968091, |
| 79152200 | | of | | | | | | | cg05099909, |
| | | ODZ4, | | | | | | | cg25965355, |
| | | promoter | | | | | | | cg14309111, |
| | | of | | | | | | | cg00908927, |
| | | TENM4 | | | | | | | cg02114107, |
| | | | | | | | | | cg19884965, |
| | | | | | | | | | cg12246510, |
| | | | | | | | | | cg03648711, |
| | | | | | | | | | cg26977644, |
| | | | | | | | | | cg12841273, |
| | | | | | | | | | cg09673208, |
| | | | | | | | | | cg01567671, |
| | | | | | | | | | cg00366359, |
| | | | | | | | | | cg22782986, |
| | | | | | | | | | cg19842216, |
| | | | | | | | | | cg04983516, |
| | | | | | | | | | cg17579825, |
| | | | | | | | | | cg03970849, |
| | | | | | | | | | cg05218311, |
| | | | | | | | | | cg11862642, |
| | | | | | | | | | cg15355859, |
| | | | | | | | | | cg02409108, |
| | | | | | | | | | cg06892009, |
| | | | | | | | | | cg26430023, |
| | | | | | | | | | cg13080602, |
| | | | | | | | | | cg05481474, |
| | | | | | | | | | cg01149449, |
| | | | | | | | | | cg15310583, |
| | | | | | | | | | cg14294793 |
| chr11: 94706291- | 20 | promoter | 0.42 | 0.00390344 | 0.031222 | 1.1275783 | 0.00022 | 0.039063184 | cg20096208, |
| 94707060 | | of | | | | | | | cg16384862, |
| | | KDM4D, | | | | | | | cg13474527, |
| | | promoter | | | | | | | cg21809762, |
| | | and | | | | | | | cg05745632, |
| | | body of | | | | | | | cg01504836, |
| | | CWC15 | | | | | | | cg04388472, |
| | | | | | | | | | cg24462596, |
| | | | | | | | | | cg24506025, |
| | | | | | | | | | cg05713782, |
| | | | | | | | | | cg14963860, |
| | | | | | | | | | cg02648738, |
| | | | | | | | | | cg21568009, |
| | | | | | | | | | cg09074260, |
| | | | | | | | | | cg16993220, |
| | | | | | | | | | cg20288268, |
| | | | | | | | | | cg12580072, |
| | | | | | | | | | cg03942286, |
| | | | | | | | | | cg22672381, |
| | | | | | | | | | cg03607513 |
| chr12: 49738680- | 20 | promoter | 0.12 | 0.00390545 | 0.031222 | 1.0935042 | 0.0002 | 0.03655387 | cg22785468, |
| 49740841 | | of | | | | | | | cg21446725, |
| | | DNAJC22 | | | | | | | cg25179358, |
| | | | | | | | | | cg14950855, |
| | | | | | | | | | cg21518937, |
| | | | | | | | | | cg07028869, |
| | | | | | | | | | cg17420360, |
| | | | | | | | | | cg25147139, |
| | | | | | | | | | cg22913903, |
| | | | | | | | | | cg14753074, |
| | | | | | | | | | cg11303127, |
| | | | | | | | | | cg27112156, |
| | | | | | | | | | cg05511977, |
| | | | | | | | | | cg05639747, |
| | | | | | | | | | cg20954975, |
| | | | | | | | | | cg09865760, |
| | | | | | | | | | cg07346931, |
| | | | | | | | | | cg19816667, |
| | | | | | | | | | cg04358741, |
| | | | | | | | | | cg15170634 |
| chr12: 57609976- | 24 | promoter | 0.40 | 0.00340812 | 0.028497 | 0.7137577 | 0.00018 | 0.035038325 | cg23853145, |
| 57611168 | | and | | | | | | | cg11468462, |
| | | body of | | | | | | | cg01606023, |
| | | NXPH4 | | | | | | | cg07159490, |
| | | | | | | | | | cg14910368, |
| | | | | | | | | | cg10701104, |
| | | | | | | | | | cg13764877, |
| | | | | | | | | | cg04186868, |
| | | | | | | | | | cg27361964, |
| | | | | | | | | | cg00818480, |
| | | | | | | | | | cg19445726, |
| | | | | | | | | | cg11441553, |
| | | | | | | | | | cg22229960, |
| | | | | | | | | | cg03921149, |
| | | | | | | | | | cg04093168, |
| | | | | | | | | | cg22061907, |
| | | | | | | | | | cg13934606, |
| | | | | | | | | | cg08699270, |
| | | | | | | | | | cg02675634, |
| | | | | | | | | | cg10541674, |
| | | | | | | | | | cg22957228, |
| | | | | | | | | | cg00969047, |
| | | | | | | | | | cg08711175, |
| | | | | | | | | | cg23047693 |
| chr13: 50697984- | 19 | promoter | 0.43 | 0.00020289 | 0.004378 | 0.8704022 | 0.00023 | 0.039262947 | cg01803928, |
| 50702286 | | and | | | | | | | cg20293942, |
| | | body of | | | | | | | cg20733077, |
| | | DLEU2 | | | | | | | cg01752594, |
| | | | | | | | | | cg01404873, |
| | | | | | | | | | cg23104954, |
| | | | | | | | | | cg15214605, |
| | | | | | | | | | cg07429908, |
| | | | | | | | | | cg12378878, |
| | | | | | | | | | cg25287268, |
| | | | | | | | | | cg26128977, |
| | | | | | | | | | cg02920897, |
| | | | | | | | | | cg20863107, |
| | | | | | | | | | cg02992881, |
| | | | | | | | | | cg03778895, |
| | | | | | | | | | cg11446099, |
| | | | | | | | | | cg06133205, |
| | | | | | | | | | cg00190330, |
| | | | | | | | | | cg17774539 |
| chr14: 61746804- | 17 | promoter | 1.93 | 1.03E−07 | 1.67E−05 | 1.1402569 | 1.68E−05 | 0.009511722 | cg10081469, |
| 61748141 | | and | | | | | | | cg12343913, |
| | | first | | | | | | | cg01084740, |
| | | exon of | | | | | | | cg10241319, |
| | | TMEM30B | | | | | | | cg00862597, |
| | | | | | | | | | cg04373359, |
| | | | | | | | | | cg01546243, |
| | | | | | | | | | cg11001769, |
| | | | | | | | | | cg15891218, |
| | | | | | | | | | cg19705215, |
| | | | | | | | | | cg04141707, |
| | | | | | | | | | cg24785368, |
| | | | | | | | | | cg01835384, |
| | | | | | | | | | cg19918763, |
| | | | | | | | | | cg18001872, |
| | | | | | | | | | cg00104086, |
| | | | | | | | | | cg10749808 |
| chr14: 61787880- | 28 | promoter | 1.43 | 2.59E−08 | 5.72E−06 | 0.8035012 | 0.00012 | 0.027784065 | cg03574415, |
| 61789467 | | and | | | | | | | cg03576946, |
| | | body of | | | | | | | cg13425637, |
| | | PRKCH | | | | | | | cg00012992, |
| | | | | | | | | | cg25370702, |
| | | | | | | | | | cg04087789, |
| | | | | | | | | | cg07555797, |
| | | | | | | | | | cg20596273, |
| | | | | | | | | | cg26470268, |
| | | | | | | | | | cg09556654, |
| | | | | | | | | | cg05538745, |
| | | | | | | | | | cg18729886, |
| | | | | | | | | | cg02121330, |
| | | | | | | | | | cg20457147, |
| | | | | | | | | | cg22530767, |
| | | | | | | | | | cg03810300, |
| | | | | | | | | | cg26157600, |
| | | | | | | | | | cg02328317, |
| | | | | | | | | | cg26590588, |
| | | | | | | | | | cg04548699, |
| | | | | | | | | | cg12165758, |
| | | | | | | | | | cg17306848, |
| | | | | | | | | | cg25562834, |
| | | | | | | | | | cg16771402, |
| | | | | | | | | | cg09991946, |
| | | | | | | | | | cg02282237, |
| | | | | | | | | | cg00244040, |
| | | | | | | | | | cg23532679 |
| chr15: 101389732- | 16 | | 0.91 | 0.00026046 | 0.023389 | 2.3389174 | 1.17E−08 | 3.81E−05 | cg09463814, |
| 101390260 | | | | | | | | | cg17221377, |
| | | | | | | | | | cg16548362, |
| | | | | | | | | | cg09785344, |
| | | | | | | | | | cg25878441, |
| | | | | | | | | | cg04392029, |
| | | | | | | | | | cg10405604, |
| | | | | | | | | | cg09747633, |
| | | | | | | | | | cg13494481, |
| | | | | | | | | | cg18304498, |
| | | | | | | | | | cg05500125, |
| | | | | | | | | | cg07035436, |
| | | | | | | | | | cg15890882, |
| | | | | | | | | | cg05000474, |
| | | | | | | | | | cg23117796, |
| | | | | | | | | | cg07882398 |
| chr15: 41217789- | 31 | promoter | 0.64 | 0.00013483 | 0.003357 | 0.5012385 | 4.12E−05 | 0.014902956 | cg07873251, |
| 41223180 | | and | | | | | | | cg08395925, |
| | | body | | | | | | | cg04946603, |
| | | DLL4 | | | | | | | cg22276692, |
| | | | | | | | | | cg07932921, |
| | | | | | | | | | cg00881300, |
| | | | | | | | | | cg12064947, |
| | | | | | | | | | cg18913798, |
| | | | | | | | | | cg06018514, |
| | | | | | | | | | cg26212303, |
| | | | | | | | | | cg16069079, |
| | | | | | | | | | cg21215323, |
| | | | | | | | | | cg01323926, |
| | | | | | | | | | cg02962630, |
| | | | | | | | | | cg00040007, |
| | | | | | | | | | cg24697497, |
| | | | | | | | | | cg20654074, |
| | | | | | | | | | cg02573468, |
| | | | | | | | | | cg00940007, |
| | | | | | | | | | cg10988513, |
| | | | | | | | | | cg07598561, |
| | | | | | | | | | cg17316580, |
| | | | | | | | | | cg04579211, |
| | | | | | | | | | cg07431199, |
| | | | | | | | | | cg13579562, |
| | | | | | | | | | cg12163955, |
| | | | | | | | | | cg16895710, |
| | | | | | | | | | cg16836355, |
| | | | | | | | | | cg03421485, |
| | | | | | | | | | cg21893456, |
| | | | | | | | | | cg22835157 |
| chr15: 71407656- | 21 | promoter | 0.69 | 0.00013107 | 0.003298 | 0.8682475 | 8.76E−06 | 0.005849883 | cg03364758, |
| 71408498 | | of | | | | | | | cg18088653, |
| | | CT62 | | | | | | | cg18581173, |
| | | | | | | | | | cg14203580, |
| | | | | | | | | | cg12637920, |
| | | | | | | | | | cg12950645, |
| | | | | | | | | | cg00316759, |
| | | | | | | | | | cg02694099, |
| | | | | | | | | | cg04988206, |
| | | | | | | | | | cg09693728, |
| | | | | | | | | | cg13125884, |
| | | | | | | | | | cg22253838, |
| | | | | | | | | | cg26401166, |
| | | | | | | | | | cg12599673, |
| | | | | | | | | | cg10175320, |
| | | | | | | | | | cg03416917, |
| | | | | | | | | | cg07097876, |
| | | | | | | | | | cg22948791, |
| | | | | | | | | | cg05415308, |
| | | | | | | | | | cg13785883, |
| | | | | | | | | | cg04963480 |
| chr15: 72522131- | 29 | promoter | 1.13 | 0.00035451 | 0.006359 | 0.6266084 | 0.00018 | 0.035497007 | cg24327132, |
| 72524238 | | of | | | | | | | cg18951187, |
| | | PKM, | | | | | | | cg14770562, |
| | | promoter | | | | | | | cg10662946, |
| | | of | | | | | | | cg00018179, |
| | | PKM2 | | | | | | | cg03989244, |
| | | | | | | | | | cg16940801, |
| | | | | | | | | | cg22129757, |
| | | | | | | | | | cg11028091, |
| | | | | | | | | | cg12433486, |
| | | | | | | | | | cg00171565, |
| | | | | | | | | | cg11471939, |
| | | | | | | | | | cg11609045, |
| | | | | | | | | | cg22171725, |
| | | | | | | | | | cg12359077, |
| | | | | | | | | | cg23160336, |
| | | | | | | | | | cg25016070, |
| | | | | | | | | | cg20070323, |
| | | | | | | | | | cg23314488, |
| | | | | | | | | | cg20909752, |
| | | | | | | | | | cg03868122, |
| | | | | | | | | | cg22234930, |
| | | | | | | | | | cg08714754, |
| | | | | | | | | | cg08053149, |
| | | | | | | | | | cg16892255, |
| | | | | | | | | | cg18321729, |
| | | | | | | | | | cg02358251, |
| | | | | | | | | | cg05888487, |
| | | | | | | | | | cg20663939 |
| chr15: 74218696- | 33 | promoter | 1.34 | 3.02E−10 | 1.46E−07 | 0.6815409 | 0.00011 | 0.026750082 | cg00313401, |
| 74220373 | | and | | | | | | | cg20652404, |
| | | body of | | | | | | | cg23484268, |
| | | LOXL1, | | | | | | | cg14435807, |
| | | promoter | | | | | | | cg16706749, |
| | | and | | | | | | | cg24168641, |
| | | body of | | | | | | | cg27554189, |
| | | LOXL1- | | | | | | | cg16394215, |
| | | AS1 | | | | | | | cg12594244, |
| | | | | | | | | | cg02812767, |
| | | | | | | | | | cg10372921, |
| | | | | | | | | | cg00527825, |
| | | | | | | | | | cg22699035, |
| | | | | | | | | | cg05241575, |
| | | | | | | | | | cg22590761, |
| | | | | | | | | | cg04024170, |
| | | | | | | | | | cg19257102, |
| | | | | | | | | | cg04436755, |
| | | | | | | | | | cg04604773, |
| | | | | | | | | | cg00071887, |
| | | | | | | | | | cg07367300, |
| | | | | | | | | | cg03682712, |
| | | | | | | | | | cg00028013, |
| | | | | | | | | | cg14849716, |
| | | | | | | | | | cg17816518, |
| | | | | | | | | | cg08372668, |
| | | | | | | | | | cg06283368, |
| | | | | | | | | | cg19087463, |
| | | | | | | | | | cg22242148, |
| | | | | | | | | | cg05388110, |
| | | | | | | | | | cg01349856, |
| | | | | | | | | | cg19036075, |
| | | | | | | | | | cg25738958 |
| chr16: 66958733- | 17 | promoter | 1.15 | 0.00173637 | 0.01804 | 0.8879075 | 0.00014 | 0.029940678 | cg20389917, |
| 66959655 | | and | | | | | | | cg09376577, |
| | | body of | | | | | | | cg24359536, |
| | | RRAD | | | | | | | cg00913604, |
| | | | | | | | | | cg26709950, |
| | | | | | | | | | cg21391551, |
| | | | | | | | | | cg09942293, |
| | | | | | | | | | cg12133305, |
| | | | | | | | | | cg05544396, |
| | | | | | | | | | cg13645565, |
| | | | | | | | | | cg17801018, |
| | | | | | | | | | cg07442105, |
| | | | | | | | | | cg19428417, |
| | | | | | | | | | cg25969900, |
| | | | | | | | | | cg01266287, |
| | | | | | | | | | cg08890824, |
| | | | | | | | | | cg00037186 |
| chr16: 68298012- | 18 | promoter | 1.03 | 1.41E−06 | 0.000111 | 1.0020001 | 5.27E−05 | 0.015776078 | cg19847229, |
| 68298979 | | of | | | | | | | cg22328890, |
| | | SLC7A6, | | | | | | | cg07273125, |
| | | 3′UTR of | | | | | | | cg09181339, |
| | | PLA2G15 | | | | | | | cg17164045, |
| | | | | | | | | | cg06327842, |
| | | | | | | | | | cg13769523, |
| | | | | | | | | | cg01291010, |
| | | | | | | | | | cg12463379, |
| | | | | | | | | | cg07925823, |
| | | | | | | | | | cg05157501, |
| | | | | | | | | | cg16859906, |
| | | | | | | | | | cg20488619, |
| | | | | | | | | | cg14886930, |
| | | | | | | | | | cg14480782, |
| | | | | | | | | | cg09194755, |
| | | | | | | | | | cg06305340, |
| | | | | | | | | | cg10049535 |
| chr16: 86539118- | 10 | | 1.20 | 4.45E−05 | 0.011876 | 1.1875647 | 0.00029 | 0.045031324 | cg01684248, |
| 86539486 | | | | | | | | | cg07865923, |
| | | | | | | | | | cg01312445, |
| | | | | | | | | | cg07136023, |
| | | | | | | | | | cg08076158, |
| | | | | | | | | | cg26657382, |
| | | | | | | | | | cg02503117, |
| | | | | | | | | | cg09998861, |
| | | | | | | | | | cg17764989, |
| | | | | | | | | | cg07060913 |
| chr17: 14204168- | 31 | promoter | 0.69 | 8.10E−07 | 7.43E−05 | 0.5653712 | 6.16E−05 | 0.017658404 | cg19814946, |
| 14207702 | | and | | | | | | | cg26418770, |
| | | body of | | | | | | | cg00179906, |
| | | HS3ST3B1, | | | | | | | cg13443605, |
| | | promoter | | | | | | | cg14016875, |
| | | and | | | | | | | cg24895178, |
| | | body of | | | | | | | cg06841262, |
| | | MGC12916 | | | | | | | cg04164190, |
| | | | | | | | | | cg14914519, |
| | | | | | | | | | cg11583981, |
| | | | | | | | | | cg17863312, |
| | | | | | | | | | cg00183916, |
| | | | | | | | | | cg16619378, |
| | | | | | | | | | cg25580342, |
| | | | | | | | | | cg27369542, |
| | | | | | | | | | cg20731875, |
| | | | | | | | | | cg09570958, |
| | | | | | | | | | cg17603502, |
| | | | | | | | | | cg06005844, |
| | | | | | | | | | cg15119650, |
| | | | | | | | | | cg00266715, |
| | | | | | | | | | cg03832440, |
| | | | | | | | | | cg17639046, |
| | | | | | | | | | cg20152539, |
| | | | | | | | | | cg09172659, |
| | | | | | | | | | cg13855261, |
| | | | | | | | | | cg26572811, |
| | | | | | | | | | cg22000330, |
| | | | | | | | | | cg12103626, |
| | | | | | | | | | cg05324982, |
| | | | | | | | | | cg05160228 |
| chr17: 1952919- | 84 | promoter | 0.17 | 0.00155283 | 0.016624 | 0.8775565 | 1.03E−14 | 2.68E−10 | cg11190071, |
| 1962328 | | and | | | | | | | cg19405854, |
| | | body of | | | | | | | cg05209078, |
| | | HIC1, | | | | | | | cg18124917, |
| | | promoter | | | | | | | cg00911794, |
| | | of | | | | | | | cg25432975, |
| | | MIR212, | | | | | | | cg12255698, |
| | | promoter | | | | | | | cg17029019, |
| | | of | | | | | | | cg01160692, |
| | | MIR132, | | | | | | | cg20682981, |
| | | body | | | | | | | cg16048942, |
| | | and | | | | | | | cg20664636, |
| | | 3′UTR of | | | | | | | cg12549595, |
| | | SMG6 | | | | | | | cg01070078, |
| | | | | | | | | | cg13915354, |
| | | | | | | | | | cg10948797, |
| | | | | | | | | | cg25440818, |
| | | | | | | | | | cg25520679, |
| | | | | | | | | | cg01389917, |
| | | | | | | | | | cg09633973, |
| | | | | | | | | | cg14610962, |
| | | | | | | | | | cg13254898, |
| | | | | | | | | | cg23882658, |
| | | | | | | | | | cg21556389, |
| | | | | | | | | | cg01143579, |
| | | | | | | | | | cg19962565, |
| | | | | | | | | | cg16011800, |
| | | | | | | | | | cg23621097, |
| | | | | | | | | | cg14294250, |
| | | | | | | | | | cg26011438, |
| | | | | | | | | | cg21854952, |
| | | | | | | | | | cg03455986, |
| | | | | | | | | | cg15043785, |
| | | | | | | | | | cg17182507, |
| | | | | | | | | | cg10848624, |
| | | | | | | | | | cg00815093, |
| | | | | | | | | | cg04414274, |
| | | | | | | | | | cg00940313, |
| | | | | | | | | | cg02342533, |
| | | | | | | | | | cg05744073, |
| | | | | | | | | | cg17739038, |
| | | | | | | | | | cg02151609, |
| | | | | | | | | | cg25449542, |
| | | | | | | | | | cg24576620, |
| | | | | | | | | | cg21994267, |
| | | | | | | | | | cg06065141, |
| | | | | | | | | | cg22690984, |
| | | | | | | | | | cg13951527, |
| | | | | | | | | | cg03542428, |
| | | | | | | | | | cg02756676, |
| | | | | | | | | | cg01168201, |
| | | | | | | | | | cg00927777, |
| | | | | | | | | | cg00138101, |
| | | | | | | | | | cg14809226, |
| | | | | | | | | | cg11144056, |
| | | | | | | | | | cg22151941, |
| | | | | | | | | | cg00592510, |
| | | | | | | | | | cg05945782, |
| | | | | | | | | | cg19001794, |
| | | | | | | | | | cg25365746, |
| | | | | | | | | | cg22934970, |
| | | | | | | | | | cg05445638, |
| | | | | | | | | | cg02376827, |
| | | | | | | | | | cg13389502, |
| | | | | | | | | | cg21810173, |
| | | | | | | | | | cg25893992, |
| | | | | | | | | | cg22208012, |
| | | | | | | | | | cg19058189, |
| | | | | | | | | | cg04631281, |
| | | | | | | | | | cg05775675, |
| | | | | | | | | | cg18051461, |
| | | | | | | | | | cg17416280, |
| | | | | | | | | | cg17171962, |
| | | | | | | | | | cg24045832, |
| | | | | | | | | | cg21973370, |
| | | | | | | | | | cg01070985, |
| | | | | | | | | | cg24173182, |
| | | | | | | | | | cg17210604, |
| | | | | | | | | | cg00572843, |
| | | | | | | | | | cg03244036, |
| | | | | | | | | | cg03978498, |
| | | | | | | | | | cg18758230, |
| | | | | | | | | | cg10530104, |
| | | | | | | | | | cg02964474 |
| chr17: 26925742- | 16 | promoter | 0.98 | 0.00037669 | 0.006607 | 1.2783942 | 0.00011 | 0.026803816 | cg01724566, |
| 26926512 | | of | | | | | | | cg18182575, |
| | | SPAG5, | | | | | | | cg25075870, |
| | | promoter | | | | | | | cg06774283, |
| | | and | | | | | | | cg01626899, |
| | | body of | | | | | | | cg06329022, |
| | | SPAG5- | | | | | | | cg27382861, |
| | | AS1 | | | | | | | cg04767934, |
| | | | | | | | | | cg00449941, |
| | | | | | | | | | cg17774070, |
| | | | | | | | | | cg08062469, |
| | | | | | | | | | cg25755953, |
| | | | | | | | | | cg06803850, |
| | | | | | | | | | cg23395533, |
| | | | | | | | | | cg20155566, |
| | | | | | | | | | cg17960080 |
| chr17: 48585385- | 18 | promoter | 1.20 | 1.16E−05 | 0.000554 | 1.9663043 | 2.12E−12 | 2.76E−08 | cg20138264, |
| 48586167 | | and | | | | | | | cg09265274, |
| | | body of | | | | | | | cg00810055, |
| | | MYCBPAP | | | | | | | cg20111217, |
| | | | | | | | | | cg22571038, |
| | | | | | | | | | cg03611598, |
| | | | | | | | | | cg11440486, |
| | | | | | | | | | cg03661110, |
| | | | | | | | | | cg10251190, |
| | | | | | | | | | cg06086634, |
| | | | | | | | | | cg01697487, |
| | | | | | | | | | cg24977335, |
| | | | | | | | | | cg20120165, |
| | | | | | | | | | cg03168497, |
| | | | | | | | | | cg27403810, |
| | | | | | | | | | cg00901687, |
| | | | | | | | | | cg09404642, |
| | | | | | | | | | cg02788401 |
| chr17: 48636103- | 46 | promoter | 0.38 | 0.0030409 | 0.026392 | 0.4479831 | 0.00013 | 0.029074653 | cg23614229, |
| 48639279 | | and | | | | | | | cg10383028, |
| | | body of | | | | | | | cg25886457, |
| | | CACNA1G, | | | | | | | cg20467136, |
| | | promoter | | | | | | | cg26892115, |
| | | and | | | | | | | cg02344539, |
| | | body of | | | | | | | cg26619156, |
| | | CACNA1G- | | | | | | | cg08133931, |
| | | AS1, | | | | | | | cg18337803, |
| | | body | | | | | | | cg01620849, |
| | | and | | | | | | | cg08917429, |
| | | 3′UTR of | | | | | | | cg23599559, |
| | | SPATA20 | | | | | | | cg12573516, |
| | | | | | | | | | cg01507046, |
| | | | | | | | | | cg24280645, |
| | | | | | | | | | cg09376537, |
| | | | | | | | | | cg09529433, |
| | | | | | | | | | cg11071401, |
| | | | | | | | | | cg27426707, |
| | | | | | | | | | cg21785145, |
| | | | | | | | | | cg16068336, |
| | | | | | | | | | cg20811659, |
| | | | | | | | | | cg14261472, |
| | | | | | | | | | cg11262815, |
| | | | | | | | | | cg19450714, |
| | | | | | | | | | cg16829453, |
| | | | | | | | | | cg14315444, |
| | | | | | | | | | cg02146257, |
| | | | | | | | | | cg27390596, |
| | | | | | | | | | cg09135695, |
| | | | | | | | | | cg15033031, |
| | | | | | | | | | cg15017244, |
| | | | | | | | | | cg01811187, |
| | | | | | | | | | cg18454685, |
| | | | | | | | | | cg04778194, |
| | | | | | | | | | cg09824855, |
| | | | | | | | | | cg03141709, |
| | | | | | | | | | cg12653738, |
| | | | | | | | | | cg01157003, |
| | | | | | | | | | cg16766889, |
| | | | | | | | | | cg17301311, |
| | | | | | | | | | cg18318818, |
| | | | | | | | | | cg05942574, |
| | | | | | | | | | cg09744022, |
| | | | | | | | | | cg13438549, |
| | | | | | | | | | cg04216597 |
| chr17: 74706465- | 15 | promoter | 1.01 | 0.00027218 | 0.005306 | 1.1080732 | 1.37E−05 | 0.00829774 | cg22195176, |
| 74707067 | | and | | | | | | | cg07851243, |
| | | body of | | | | | | | cg16428008, |
| | | MXRA7, | | | | | | | cg12472603, |
| | | 3′UTR of | | | | | | | cg14042121, |
| | | JMJD6 | | | | | | | cg20832875, |
| | | | | | | | | | cg20546985, |
| | | | | | | | | | cg17185586, |
| | | | | | | | | | cg04880618, |
| | | | | | | | | | cg11122493, |
| | | | | | | | | | cg07484485, |
| | | | | | | | | | cg10267491, |
| | | | | | | | | | cg22216643, |
| | | | | | | | | | cg20730545, |
| | | | | | | | | | cg15769653 |
| chr18: 24126780- | 36 | promoter | 0.68 | 7.13E−06 | 0.00038 | 0.6478899 | 9.55E−05 | 0.024625762 | cg03740978, |
| 24131138 | | of | | | | | | | cg19738924, |
| | | KCTD1 | | | | | | | cg05176991, |
| | | | | | | | | | cg00044299, |
| | | | | | | | | | cg13181251, |
| | | | | | | | | | cg01065003, |
| | | | | | | | | | cg00868875, |
| | | | | | | | | | cg06844968, |
| | | | | | | | | | cg12776287, |
| | | | | | | | | | cg12075497, |
| | | | | | | | | | cg20728364, |
| | | | | | | | | | cg24522982, |
| | | | | | | | | | cg23777946, |
| | | | | | | | | | cg05840573, |
| | | | | | | | | | cg08057338, |
| | | | | | | | | | cg12918961, |
| | | | | | | | | | cg06206801, |
| | | | | | | | | | cg16547027, |
| | | | | | | | | | cg10301338, |
| | | | | | | | | | cg26961386, |
| | | | | | | | | | cg02173326, |
| | | | | | | | | | cg03818793, |
| | | | | | | | | | cg02901177, |
| | | | | | | | | | cg18757695, |
| | | | | | | | | | cg18377217, |
| | | | | | | | | | cg10096645, |
| | | | | | | | | | cg00702546, |
| | | | | | | | | | cg26704078, |
| | | | | | | | | | cg24045369, |
| | | | | | | | | | cg24965080, |
| | | | | | | | | | cg12881557, |
| | | | | | | | | | cg12851609, |
| | | | | | | | | | cg20002283, |
| | | | | | | | | | cg05800683, |
| | | | | | | | | | cg07965447, |
| | | | | | | | | | cg20382774 |
| chr18: 30349690- | 25 | promoter | 1.10 | 8.35E−08 | 1.40E−05 | 0.9334036 | 5.34E−09 | 1.99E−05 | cg22648949, |
| 30352302 | | and | | | | | | | cg05134926, |
| | | body of | | | | | | | cg09381134, |
| | | KLHL14 | | | | | | | cg17196268, |
| | | | | | | | | | cg03513246, |
| | | | | | | | | | cg16967099, |
| | | | | | | | | | cg14891195, |
| | | | | | | | | | cg16016270, |
| | | | | | | | | | cg03477049, |
| | | | | | | | | | cg16342115, |
| | | | | | | | | | cg06869709, |
| | | | | | | | | | cg08701601, |
| | | | | | | | | | cg18774642, |
| | | | | | | | | | cg16501308, |
| | | | | | | | | | cg04209727, |
| | | | | | | | | | cg13353999, |
| | | | | | | | | | cg01679516, |
| | | | | | | | | | cg21501358, |
| | | | | | | | | | cg05784157, |
| | | | | | | | | | cg13476901, |
| | | | | | | | | | cg06485671, |
| | | | | | | | | | cg20162206, |
| | | | | | | | | | cg01472737, |
| | | | | | | | | | cg06591973, |
| | | | | | | | | | cg27014538 |
| chr19: 1465206- | 21 | body of | 0.68 | 0.00033055 | 0.006055 | 1.3128307 | 9.50E−08 | 0.000190322 | cg10090761, |
| 1471241 | | APC2 | | | | | | | cg08958549, |
| | | | | | | | | | cg13368085, |
| | | | | | | | | | cg12154045, |
| | | | | | | | | | cg24883899, |
| | | | | | | | | | cg03306486, |
| | | | | | | | | | cg10565187, |
| | | | | | | | | | cg02574474, |
| | | | | | | | | | cg19305488, |
| | | | | | | | | | cg10169241, |
| | | | | | | | | | cg05457563, |
| | | | | | | | | | cg04624885, |
| | | | | | | | | | cg05620923, |
| | | | | | | | | | cg12400781, |
| | | | | | | | | | cg19333963, |
| | | | | | | | | | cg04203646, |
| | | | | | | | | | cg06346838, |
| | | | | | | | | | cg06508886, |
| | | | | | | | | | cg10094078, |
| | | | | | | | | | cg22560193, |
| | | | | | | | | | cg15709766 |
| chr19: 34012271- | 17 | promoter | 0.55 | 0.00119936 | 0.014012 | 0.8538192 | 0.00011 | 0.026750082 | cg15520477, |
| 34012936 | | of | | | | | | | cg02300764, |
| | | PEPD | | | | | | | cg06546607, |
| | | | | | | | | | cg18394714, |
| | | | | | | | | | cg01371108, |
| | | | | | | | | | cg19385386, |
| | | | | | | | | | cg17811310, |
| | | | | | | | | | cg18701660, |
| | | | | | | | | | cg22513356, |
| | | | | | | | | | cg25698525, |
| | | | | | | | | | cg13993643, |
| | | | | | | | | | cg23010452, |
| | | | | | | | | | cg08085561, |
| | | | | | | | | | cg07603357, |
| | | | | | | | | | cg17533158, |
| | | | | | | | | | cg23519308, |
| | | | | | | | | | cg10010386 |
| chr19: 46916587- | 11 | promoter | 1.15 | 0.00134375 | 0.01506 | 1.6254384 | 0.00014 | 0.029372897 | cg15984661, |
| 46916862 | | of | | | | | | | cg20265803, |
| | | CCDC8 | | | | | | | cg25987744, |
| | | | | | | | | | cg23085676, |
| | | | | | | | | | cg15023922, |
| | | | | | | | | | cg02512703, |
| | | | | | | | | | cg20071868, |
| | | | | | | | | | cg06747432, |
| | | | | | | | | | cg11125714, |
| | | | | | | | | | cg23039227, |
| | | | | | | | | | cg09411654 |
| chr19: 47922251- | 17 | promoter | 0.58 | 0.00093566 | 0.011923 | 0.7974177 | 2.32E−05 | 0.010789657 | cg21145624, |
| 47922777 | | and | | | | | | | cg17027233, |
| | | body of | | | | | | | cg24494876, |
| | | MEIS3 | | | | | | | cg26502429, |
| | | | | | | | | | cg08810007, |
| | | | | | | | | | cg07499197, |
| | | | | | | | | | cg07240206, |
| | | | | | | | | | cg07021268, |
| | | | | | | | | | cg13822158, |
| | | | | | | | | | cg04589660, |
| | | | | | | | | | cg13275680, |
| | | | | | | | | | cg16969934, |
| | | | | | | | | | cg10480476, |
| | | | | | | | | | cg21722128, |
| | | | | | | | | | cg02141602, |
| | | | | | | | | | cg22454370, |
| | | | | | | | | | cg06028671 |
| chr19: 496158- | 10 | promoter | 0.69 | 0.00205732 | 0.020137 | 1.1464837 | 5.68E−05 | 0.016621339 | cg08403345, |
| 496481 | | of | | | | | | | cg14985989, |
| | | MADCAM1 | | | | | | | cg13777292, |
| | | | | | | | | | cg21796096, |
| | | | | | | | | | cg26525091, |
| | | | | | | | | | cg06706875, |
| | | | | | | | | | cg14045283, |
| | | | | | | | | | cg04139060, |
| | | | | | | | | | cg26522278, |
| | | | | | | | | | cg17370697 |
| chr19: 50931270- | 9 | body | 2.11 | 0.00028658 | 0.005505 | 2.1457641 | 2.55E−05 | 0.011256305 | cg21152077, |
| 50931638 | | and | | | | | | | cg19387862, |
| | | 3′UTR of | | | | | | | cg13403724, |
| | | SPIB | | | | | | | cg15007959, |
| | | | | | | | | | cg22745102, |
| | | | | | | | | | cg24092179, |
| | | | | | | | | | cg04508467, |
| | | | | | | | | | cg15690347, |
| | | | | | | | | | cg16550154 |
| chr20: 37230523- | 12 | promoter | 1.09 | 4.31E−05 | 0.001493 | 1.4610661 | 1.43E−05 | 0.0084643 | cg13715798, |
| 37230742 | | and | | | | | | | cg14040722, |
| | | body of | | | | | | | cg08438366, |
| | | C20orf95, | | | | | | | cg09533655, |
| | | promoter | | | | | | | cg06301550, |
| | | of | | | | | | | cg13523649, |
| | | ARHGAP40 | | | | | | | cg03356734, |
| | | | | | | | | | cg10344023, |
| | | | | | | | | | cg02027735, |
| | | | | | | | | | cg01025836, |
| | | | | | | | | | cg07159871, |
| | | | | | | | | | cg26118446 |
| chr21: 34395128- | 34 | promoter | 0.44 | 2.17E−06 | 0.000153 | 0.5756563 | 0.00025 | 0.040838476 | cg18374181, |
| 34400245 | | and | | | | | | | cg10364942, |
| | | body of | | | | | | | cg14730102, |
| | | OLIG2 | | | | | | | cg11215918, |
| | | | | | | | | | cg16713743, |
| | | | | | | | | | cg02858594, |
| | | | | | | | | | cg21032292, |
| | | | | | | | | | cg06515159, |
| | | | | | | | | | cg03861097, |
| | | | | | | | | | cg22593533, |
| | | | | | | | | | cg11950383, |
| | | | | | | | | | cg17013986, |
| | | | | | | | | | cg05238769, |
| | | | | | | | | | cg25661973, |
| | | | | | | | | | cg08729810, |
| | | | | | | | | | cg02115911, |
| | | | | | | | | | cg08358474, |
| | | | | | | | | | cg07601542, |
| | | | | | | | | | cg02965237, |
| | | | | | | | | | cg16403860, |
| | | | | | | | | | cg14843922, |
| | | | | | | | | | cg15299832, |
| | | | | | | | | | cg14293300, |
| | | | | | | | | | cg03696345, |
| | | | | | | | | | cg05634149, |
| | | | | | | | | | cg02100602, |
| | | | | | | | | | cg27254482, |
| | | | | | | | | | cg05724110, |
| | | | | | | | | | cg08870743, |
| | | | | | | | | | cg10217445, |
| | | | | | | | | | Cg27357571, |
| | | | | | | | | | cg13524919, |
| | | | | | | | | | cg10829693, |
| | | | | | | | | | cg23253569 |
| chr21: 46785130- | 10 | | 1.26 | 0.00030982 | 0.005764 | 1.1522913 | 0.0003 | 0.045570045 | cg15140798, |
| 46785339 | | | | | | | | | cg26958236, |
| | | | | | | | | | cg06868026, |
| | | | | | | | | | cg09476092, |
| | | | | | | | | | cg12098784, |
| | | | | | | | | | cg14899046, |
| | | | | | | | | | cg18087395, |
| | | | | | | | | | cg24975688, |
| | | | | | | | | | cg20383624, |
| | | | | | | | | | cg20152484 |
| chr22: 32339933- | 29 | promoter | 1.12 | 1.29E−09 | 4.98E−07 | 0.9340402 | 4.74E−06 | 0.003857768 | cg25983317, |
| 32341192 | | and | | | | | | | cg12700033, |
| | | body of | | | | | | | cg03814826, |
| | | YWHAH, | | | | | | | cg07137170, |
| | | promoter | | | | | | | cg15233292, |
| | | and | | | | | | | cg05856065, |
| | | body of | | | | | | | cg18330041, |
| | | C22orf24, | | | | | | | cg01002120, |
| | | | | | | | | | cg18068862, |
| | | | | | | | | | cg20306180, |
| | | | | | | | | | cg16001977, |
| | | | | | | | | | cg12752956, |
| | | | | | | | | | cg00907604, |
| | | | | | | | | | cg05946971, |
| | | | | | | | | | cg02457501, |
| | | | | | | | | | cg06131547, |
| | | | | | | | | | cg26914705, |
| | | | | | | | | | cg05128038, |
| | | | | | | | | | cg12624087, |
| | | | | | | | | | cg06464744, |
| | | | | | | | | | cg12003043, |
| | | | | | | | | | cg15644389, |
| | | | | | | | | | cg01616215, |
| | | | | | | | | | cg06462684, |
| | | | | | | | | | cg19764325, |
| | | | | | | | | | cg24968946, |
| | | | | | | | | | cg10984950, |
| | | | | | | | | | cg22478328, |
| | | | | | | | | | cg00455418 |

### Example 4. Expression Changes Due to Ischemia-Induced Hypermethylation

Interestingly, pathway analysis on the 81 genes associated with these 66 CpG islands revealed that genes involved in the negative regulation of the Notch and Wnt pathway, which are strongly implicated in kidney fibrosis and allograft injuryl⁴, were enriched (FIG. 5A)¹³. Other genes also played a role in the negative regulation of apoptosis and cell death (FIG. 5B).

To evaluate hypermethylation of these 66 CpG islands also translates into gene expression changes within the allograft, we evaluated expression of the corresponding genes in the paired pre- versus post-ischemia biopsies of the longitudinal cohort. Of the 65 genes for which we could reliably assess expression changes, 55 (84.6%) were characterized by decreased expression in kidney transplants upon ischemia and reperfusion (29 at P<0.05, FIG. 5C). These 29 CpG islands were mainly located in gene promoters, consistent with hypermethylation suppressing gene expression. Three genes (MSX1, RRAD and DLL4) were characterized by increased expression (P<0.05), but the corresponding hypermethylated CpG islands overlapped either completely (MSX1) or partly (RRAD, DLL4) with gene bodies. Overall, these findings indicate that methylation occurring upon ischemia affects genes in biologically relevant pathways and mostly decreases expression of the associated gene.

### Example 5. Ischemia-Induced Hypermethylation and Chronic Allograft Injury

Next, we assessed whether these methylation changes become transient or stably imbedded in the kidney methylome after the ischemic insult. We measured DNA methylation in biopsies obtained several months after transplantation (longitudinal cohort) and assessed hypermethylation in the 66 CpG islands. Interestingly, we observed that CpGs located in these islands were still hypermethylated at 3 months and 1 year after transplantation (FIG. 6A).

We then investigated whether ischemia-induced hypermethylation observed at the time of transplantation correlates with chronic allograft injury (calculated by the Chronic Allograft Damage Index (CADI) score¹⁴) (Table 1). When correlating the methylation status of 1 634 CpGs in the 66 islands with injury, we found that 487 (30%) and 332 (20%) CpGs were positively correlated with CADI score at 3 months, respectively at P<0.05 and FDR<0.05, whereas 402 (25%) and 135 (8%) CpGs were associated with CADI at 1 year. This was significantly more than the 48 and 14 CpGs negatively correlating (P<0.05) with CADI at 3 months and 1 year, respectively. When adjusting for donor age and gender, similar effects were observed. The bias towards a direct correlation between hypermethylation and future injury was also not detected at baseline injury, as only 43 out of 75 (57%; P>0.05) CpGs correlated positively with CADI at baseline. Also when adjusting for cold and warm ischemia time, DNA methylation correlated better with future injury than with injury already evident at the time of transplantation.

### Example 6. DNA Hypermethylation Predicts Chronic Allograft Injury

Having shown that ischemia-induced hypermethylation of kidney transplants correlates with chronic allograft injury, we tested whether a methylation-based risk score at the time of transplantation could predict chronic injury 1 year after transplantation. The latter was defined by a CADI>2, representing a threshold that predicts graft survival at 1 year after transplantation¹⁴. First, we developed a risk score reflecting DNA methylation in the 66 CpG islands weighted for their correlation with chronic injury at one year after transplant in the pre-implantation cohort. Patients with a methylation risk score (MRS) in the highest tertile had an increased risk (odds ratio [OR], 45; 95% confidence interval [95% CI], 8 to 499; P<0.00001) to develop chronic injury relative to patients in the lowest tertile (FIG. 6, B and E). The score had an AUC value of 0.919 to predict chronic injury, thereby outperforming baseline clinical risk factors including donor age and donor criteria, donor last serum creatinine, cold ischemia time, anastomosis time and the number of HLA mismatches (combined AUC of 0.743, FIG. 6C). Since CADI combines 6 different histopathological lesions, we additionally evaluated MRS for each lesion individually. MRS was higher in recipients with interstitial fibrosis (P<0.00001), vascular intima thickening (P=0.003) and glomerulosclerosis (P=0.0001) on the 1-year protocol-specified biopsies. In contrast, MRS did not differ in recipients with or without inflammation (P=0.82), tubular atrophy (P=0.13) or mesangial matrix increase (P=0.77).

Second, we validated our MRS in an independent cross-sectional cohort of 46 post-reperfusion brain-dead donor kidney biopsies (Table 1). We deliberately selected biopsies taken at the post-reperfusion time point, which is a later time point than for the previous 2 cohorts, to ensure robustness and clinical validity of our observations. The highest versus lowest tertile of patients had an 9-fold increased risk to develop chronic injury (95% CI, 2 to 57; P=0.005, FIG. 6 B and F). Likewise, MRS yielded a better AUC than baseline clinical risk factors combined (AUC 0.775 versus 0.694, FIG. 6D). Interestingly, MRS also correlated with reduced allograft function at 1 year after transplantation (pre-implantation cohort: Pearson correlation or r=−0.29, P=0.03; post-reperfusion cohort: r=−0.37, P=0.009; FIG. 6, G and H), further strengthening the clinical significance of our findings.

### Example 7. Ranking of Methylated CpGs Based on a LASSO Model of 1000 Iterations to Predict Outcome for CAI

The methylation risk score (MRS) as used in the presented examples was developed and calculated based on the methylated CpGs listed for the 66 validated CpG islands, as shown above and in Table 2. To determine the number of CpGs that is minimally required to calculate an MRS with a better predictive power than the current clinical parameters, we used a LASSO model consisting of 1000 iterations to calculate the MRS based on as little CpGs as possible. Those minimal models were subsequently tested in the validation cohort to allow prediction of chronic allograft injury at one year after transplantation.

Instead of using 1634 methylated CpGs located within the 66 CpG islands (Table 2), only 413 different CpGs turned out to be relevant in the LASSO model (Table 3). The number of times that each of these 413 CpG was used in one of the 1000 LASSO models was used to rank the CpGs according to their importance in predicting the risk for chronic allograft injury via MRS (FIG. 7, Table 5). Of those 413 CpGs, only 29 CpGs were used in at least 10% (100 out of 1000) of the Lasso models (Table 4), and 169 CpGs were used for the MRS in 1% of the models. Finally, from these 1000 minimal models we can conclude that even 4 CpGs from the most highly-ranked CpGs (Table 4) were sufficient to acquire an MRS outperforming the clinical parameters of the validation cohort to predict chronic injury at one year after transplantation.

### Table 3. List of CpGs and Annotation for the Methylated CpGs Used in the 1000 Minimal LASSO Models.

| | No of | | | | | | | |
|---|---|---|---|---|---|---|---|---|
| CpG | times used | Percentage | chr | pos | strand | Islands_Name | Relation_to_Island | UCSC_RefGene_Name |
| cg01811187 | 767 | 76.70% | chr17 | 48637445 | + | chr17: 48636103-48639279 | Island | CACNA1G |
| cg17078427 | 703 | 70.30% | chr3 | 170137552 | − | chr3: 170136242-170137886 | Island | CLDN11 |
| cg16547027 | 462 | 46.20% | chr18 | 24127588 | − | chr18: 24126780-24131138 | Island | KCTD1 |
| cg19596468 | 458 | 45.80% | chr4 | 4864110 | + | chr4: 4864456-4864834 | N_Shore | MSX1 |
| cg14309111 | 430 | 43.00% | chr11 | 79150411 | + | chr11: 79148358-79152200 | Island | ODZ4 |
| cg17603502 | 415 | 41.50% | chr17 | 14204056 | − | chr17: 14204168-14207702 | N_Shore | HS3ST3B1 |
| cg08133931 | 384 | 38.40% | chr17 | 48636626 | + | chr17: 48636103-48639279 | Island | |
| cg18599069 | 342 | 34.20% | chr10 | 8096991 | + | chr10: 8091374-8098329 | Island | GATA3 |
| cg24840099 | 239 | 23.90% | chr4 | 4864430 | + | chr4: 4864456-4864834 | N_Shore | MSX1 |
| cg09529433 | 220 | 22.00% | chr17 | 48637255 | + | chr17: 48636103-48639279 | Island | CACNA1G |
| cg10096645 | 220 | 22.00% | chr18 | 24130851 | + | chr18: 24126780-24131138 | Island | KCTD1 |
| cg06108383 | 211 | 21.10% | chr6 | 32120899 | − | chr6: 32121829-32122529 | N_Shore | PPT2; PRRT1 |
| cg03884082 | 172 | 17.20% | chr1 | 19971709 | + | chr1: 19970255-19971923 | Island | NBL1 |
| cg01065003 | 171 | 17.10% | chr18 | 24130839 | − | chr18: 24126780-24131138 | Island | KCTD1 |
| cg22647713 | 168 | 16.80% | chr10 | 8095697 | − | chr10: 8091374-8098329 | Island | FLJ45983; GATA3 |
| cg20449692 | 162 | 16.20% | chr3 | 170136920 | − | chr3: 170136242-170137886 | Island | CLDN11 |
| cg07136023 | 150 | 15.00% | chr16 | 86537316 | − | chr16: 86539118-86539486 | N_Shore | |
| cg20811659 | 136 | 13.60% | chr17 | 48637730 | − | chr17: 48636103-48639279 | Island | CACNA1G |
| cg20048434 | 132 | 13.20% | chr10 | 116163160 | − | chr10: 116163391-116164599 | N_Shore | AFAP1L2 |
| cg06546607 | 127 | 12.70% | chr19 | 34013019 | + | chr19: 34012271-34012936 | S_Shore | PEPD |
| cg00403498 | 127 | 12.70% | chr6 | 32119923 | − | chr6: 32121829-32122529 | N_Shore | PRRT1; PPT2 |
| cg20891301 | 119 | 11.90% | chr4 | 4864711 | − | chr4: 4864456-4864834 | Island | MSX1 |
| cg17416730 | 116 | 11.60% | chr6 | 33245541 | − | chr6: 33244677-33245554 | Island | B3GALT4 |
| cg01724566 | 113 | 11.30% | chr17 | 26926132 | + | chr17: 26925742-26926512 | Island | SPAG5 |
| cg16501308 | 112 | 11.20% | chr18 | 30350221 | − | chr18: 30349690-30352302 | Island | KLHL14 |
| cg06230736 | 109 | 10.90% | chr10 | 8096650 | + | chr10: 8091374-8098329 | Island | FLJ45983; GATA3 |
| cg03199651 | 105 | 10.50% | chr4 | 4862770 | − | chr4: 4864456-4864834 | N_Shore | MSX1 |
| cg06329022 | 103 | 10.30% | chr17 | 26926511 | + | chr17: 26925742-26926512 | Island | SPAG5 |
| cg13879776 | 102 | 10.20% | chr3 | 170136263 | − | chr3: 170136242-170137886 | Island | CLDN11 |
| cg09024124 | 97 | 9.70% | chr3 | 128207255 | − | chr3: 128205495-128212274 | Island | GATA2 |
| cg01507046 | 96 | 9.60% | chr17 | 48637818 | − | chr17: 48636103-48639279 | Island | CACNA1G |
| cg17113856 | 96 | 9.60% | chr6 | 32120895 | − | chr6: 32121829-32122529 | N_Shore | PPT2; PRRT1 |
| cg07846167 | 94 | 9.40% | chr1 | 16084758 | − | chr1: 16085147-16085862 | N_Shore | FBLIM1 |
| cg18701660 | 85 | 8.50% | chr19 | 34012935 | − | chr19: 34012271-34012936 | Island | PEPD |
| cg07516470 | 82 | 8.20% | chr10 | 8095651 | − | chr10: 8091374-8098329 | Island | FLJ45983; GATA3 |
| cg21096399 | 82 | 8.20% | chr11 | 119188145 | + | chr11: 119186947-119187894 | S_Shore | MCAM |
| cg18187680 | 77 | 7.70% | chr10 | 8095825 | − | chr10: 8091374-8098329 | Island | FLJ45983; GATA3 |
| cg16519300 | 76 | 7.60% | chr1 | 16084830 | − | chr1: 16085147-16085862 | N_Shore | FBLIM1 |
| cg06375949 | 75 | 7.50% | chr4 | 4863356 | − | chr4: 4864456-4864834 | N_Shore | MSX1 |
| cg22590761 | 73 | 7.30% | chr15 | 74218921 | + | chr15: 74218696-74220373 | Island | LOXL1 |
| cg26292521 | 70 | 7.00% | chr10 | 8095831 | − | chr10: 8091374-8098329 | Island | FLJ45983; GATA3 |
| cg00110832 | 69 | 6.90% | chr6 | 32121130 | − | chr6: 32121829-32122529 | N_Shore | PPT2PRRT1 |
| cg04255616 | 67 | 6.70% | chr8 | 41167278 | + | chr8: 41165852-41167140 | S_Shore | SFRP1 |
| cg27426707 | 67 | 6.70% | chr17 | 48639585 | + | chr17: 48636103-48639279 | S_Shore | CACNA1G |
| cg24605046 | 66 | 6.60% | chr6 | 33245895 | − | chr6: 33244677-33245554 | S_Shore | B3GALT4 |
| cg12883279 | 62 | 6.20% | chr6 | 32120773 | + | chr6: 32121829-32122529 | N_Shore | PPT2; PRRT1 |
| cg18454685 | 62 | 6.20% | chr17 | 48639239 | + | chr17: 48636103-48639279 | Island | CACNA1G |
| cg25426302 | 62 | 6.20% | chr6 | 32120826 | − | chr6: 32121829-32122529 | N_Shore | PPT2; PRRT1 |
| cg16650717 | 61 | 6.10% | chr1 | 19970334 | − | chr1: 19970255-19971923 | Island | NBL1 |
| cg26270195 | 61 | 6.10% | chr6 | 33245553 | − | chr6: 33244677-33245554 | Island | B3GALT4 |
| cg00449941 | 60 | 6.00% | chr17 | 26926011 | + | chr17: 26925742-26926512 | Island | SPAG5 |
| cg23058185 | 60 | 6.00% | chr10 | 8095985 | − | chr10: 8091374-8098329 | Island | FLJ45983; GATA3 |
| cg03970849 | 59 | 5.90% | chr11 | 79148183 | − | chr11: 79148358-79152200 | N_Shore | ODZ4 |
| cg09998861 | 58 | 5.80% | chr16 | 86538106 | − | chr16: 86539118-86539486 | N_Shore | |
| cg19315863 | 56 | 5.60% | chr10 | 8096597 | + | chr10: 8091374-8098329 | Island | FLJ45983; GATA3 |
| cg17960080 | 55 | 5.50% | chr17 | 26926868 | − | chr17: 26925742-26926512 | S_Shore | SPAG5 |
| cg12163955 | 53 | 5.30% | chr15 | 41217556 | − | chr15: 41217789-41223180 | N_Shore | |
| cg06206801 | 52 | 5.20% | chr18 | 24131379 | − | chr18: 24126780-24131138 | S_Shore | KCTD1 |
| cg06803850 | 51 | 5.10% | chr17 | 26926738 | + | chr17: 26925742-26926512 | S_Shore | SPAG5 |
| cg10049535 | 51 | 5.10% | chr16 | 68299128 | − | chr16: 68298012-68298979 | S_Shore | SLC7A6 |
| cg14098681 | 50 | 5.00% | chr10 | 8096818 | − | chr10: 8091374-8098329 | Island | FLJ45983; GATA3; |
| | | | | | | | | GATA3 |
| cg20652404 | 49 | 4.90% | chr15 | 74218904 | + | chr15: 74218696-74220373 | Island | LOXL1 |
| cg08238215 | 47 | 4.70% | chr2 | 66673985 | − | chr2: 66672431-66673636 | S_Shore | MEIS1 |
| cg13934406 | 47 | 4.70% | chr6 | 32120878 | + | chr6: 32121829-32122529 | N_Shore | PPT2; PRRT1 |
| cg25144207 | 47 | 4.70% | chr4 | 4864302 | + | chr4: 4864456-4864834 | N_Shore | MSX1 |
| cg25755953 | 47 | 4.70% | chr17 | 26926457 | − | chr17: 26925742-26926512 | Island | SPAG5 |
| cg24329557 | 45 | 4.50% | chr6 | 10882326 | − | chr6: 10882926-10883149 | N_Shore | GCM2 |
| cg00319655 | 43 | 4.30% | chr4 | 79473327 | − | chr4: 79472806-79473177 | S_Shore | ANXA3 |
| cg03189210 | 41 | 4.10% | chr6 | 33245474 | − | chr6: 33244677-33245554 | Island | B3GALT4 |
| cg04963480 | 40 | 4.00% | chr15 | 71408776 | + | chr15: 71407656-71408498 | S_Shore | CT62 |
| cg04262471 | 38 | 3.80% | chr6 | 33245585 | + | chr6: 33244677-33245554 | S_Shore | B3GALT4 |
| cg17182507 | 38 | 3.80% | chr17 | 1957231 | − | chr17: 1952919-1962328 | Island | HIC1 |
| cg02048416 | 37 | 3.70% | chr2 | 74782684 | + | chr2: 74781494-74782685 | Island | DOK1 |
| cg07346931 | 37 | 3.70% | chr12 | 49743523 | − | chr12: 49738680-49740841 | S_Shelf | DNAJC22 |
| cg20328456 | 37 | 3.70% | chr6 | 32121113 | − | chr6: 32121829-32122529 | N_Shore | PPT2; PRRT1 |
| cg06023994 | 36 | 3.60% | chr3 | 170137871 | + | chr3: 170136242-170137886 | Island | CLDN11 |
| cg07434518 | 36 | 3.60% | chr3 | 170136327 | + | chr3: 170136242-170137886 | Island | CLDN11 |
| cg11590420 | 36 | 3.60% | chr5 | 150051566 | − | chr5: 150051116-150052107 | Island | MYOZ3 |
| cg14176930 | 36 | 3.60% | chr6 | 10884891 | + | chr6: 10882926-10883149 | S_Shore | |
| cg15520477 | 36 | 3.60% | chr19 | 34012957 | − | chr19: 34012271-34012936 | S_Shore | PEPD |
| cg04749507 | 33 | 3.30% | chr6 | 32120203 | + | chr6: 32121829-32122529 | N_Shore | PPT2; PRRT1 |
| cg08062469 | 33 | 3.30% | chr17 | 26926627 | + | chr17: 26925742-26926512 | S_Shore | SPAG5 |
| cg12741994 | 33 | 3.30% | chr3 | 170137321 | + | chr3: 170136242-170137886 | Island | CLDN11 |
| cg19679989 | 33 | 3.30% | chr10 | 8096602 | + | chr10: 8091374-8098329 | Island | FLJ45983; GATA3 |
| cg20663200 | 33 | 3.30% | chr10 | 116163392 | − | chr10: 116163391-116164599 | Island | AFAP1L2 |
| cg23943136 | 32 | 3.20% | chr10 | 8095755 | − | chr10: 8091374-8098329 | Island | FLJ45983; GATA3 |
| cg13398291 | 31 | 3.10% | chr8 | 41166169 | − | chr8: 41165852-41167140 | Island | SFRP1 |
| cg14315444 | 31 | 3.10% | chr17 | 48636344 | − | chr17: 48636103-48639279 | Island | |
| cg23520930 | 31 | 3.10% | chr3 | 128206967 | + | chr3: 128205495-128212274 | Island | GATA2 |
| cg03682712 | 30 | 3.00% | chr15 | 74219307 | − | chr15: 74218696-74220373 | Island | LOXL1 |
| cg22880620 | 30 | 3.00% | chr6 | 56820808 | + | chr6: 56818873-56820308 | S_Shore | BEND6; DST |
| cg25987744 | 30 | 3.00% | chr19 | 46916588 | − | chr19: 46916587-46916862 | Island | CCDC8; CCDC8 |
| cg26381352 | 30 | 3.00% | chr6 | 33244799 | − | chr6: 33244677-33245554 | Island | B3GALT4 |
| cg02551743 | 29 | 2.90% | chr2 | 66673428 | − | chr2: 66672431-66673636 | Island | MEIS1 |
| cg11522683 | 29 | 2.90% | chr6 | 37501428 | + | chr6: 37503538-37504291 | N_Shelf | |
| cg02989257 | 28 | 2.80% | chr1 | 32169274 | − | chr1: 32169537-32169869 | N_Shore | COL16A1 |
| cg08707112 | 28 | 2.80% | chr10 | 8095764 | + | chr10: 8091374-8098329 | Island | FLJ45983; GATA3 |
| cg14327531 | 28 | 2.80% | chr10 | 8097331 | − | chr10: 8091374-8098329 | Island | GATA3 |
| cg23359665 | 28 | 2.80% | chr6 | 32120907 | − | chr6: 32121829-32122529 | N_Shore | PPT2; PRRT1 |
| cg00868875 | 27 | 2.70% | chr18 | 24127237 | − | chr18: 24126780-24131138 | Island | KCTD1 |
| cg21785145 | 27 | 2.70% | chr17 | 48635853 | + | chr17: 48636103-48639279 | N_Shore | |
| cg11129609 | 26 | 2.60% | chr6 | 33247250 | − | chr6: 33244677-33245554 | S_Shore | WDR46 |
| cg17566118 | 26 | 2.60% | chr10 | 8095797 | + | chr10: 8091374-8098329 | Island | FLJ45983; GATA3 |
| cg02241055 | 24 | 2.40% | chr3 | 170136766 | + | chr3: 170136242-170137886 | Island | CLDN11 |
| cg05942574 | 24 | 2.40% | chr17 | 48637104 | − | chr17: 48636103-48639279 | Island | CACNA1G |
| cg10074727 | 24 | 2.40% | chr6 | 10883105 | − | chr6: 10882926-10883149 | Island | GCM2 |
| cg01803928 | 22 | 2.20% | chr13 | 50701619 | + | chr13: 50697984-50702286 | Island | |
| cg05671070 | 22 | 2.20% | chr10 | 8095960 | − | chr10: 8091374-8098329 | Island | FLJ45983; GATA3 |
| cg12064947 | 22 | 2.20% | chr15 | 41220983 | − | chr15: 41217789-41223180 | Island | DLL4 |
| cg12730771 | 22 | 2.20% | chr10 | 8095996 | − | chr10: 8091374-8098329 | Island | FLJ45983; GATA3 |
| cg24509300 | 22 | 2.20% | chr6 | 32123034 | − | chr6: 32121829-32122529 | S_Shore | PPT2 |
| cg00086577 | 21 | 2.10% | chr6 | 32122894 | + | chr6: 32121829-32122529 | S_Shore | PPT2 |
| cg11386011 | 21 | 2.10% | chr6 | 32121156 | + | chr6: 32121829-32122529 | N_Shore | PPT2; PRRT1 |
| cg01111041 | 20 | 2.00% | chr6 | 32121055 | + | chr6: 32121829-32122529 | N_Shore | PPT2; PRRT1 |
| cg04164190 | 20 | 2.00% | chr17 | 14205456 | − | chr17: 14204168-14207702 | Island | HS3ST3B1 |
| cg07841173 | 20 | 2.00% | chr3 | 128210150 | − | chr3: 128205495-128212274 | Island | GATA2 |
| cg19657198 | 20 | 2.00% | chr10 | 8095121 | − | chr10: 8091374-8098329 | Island | FLJ45983 |
| cg20155566 | 20 | 2.00% | chr17 | 26926074 | − | chr17: 26925742-26926512 | Island | SPAG5 |
| cg23104954 | 20 | 2.00% | chr13 | 50701501 | + | chr13: 50697984-50702286 | Island | |
| cg02344539 | 19 | 1.90% | chr17 | 48637743 | + | chr17: 48636103-48639279 | Island | CACNA1G |
| cg11731114 | 19 | 1.90% | chr10 | 8096064 | + | chr10: 8091374-8098329 | Island | FLJ45983; GATA3; |
| cg03696345 | 18 | 1.80% | chr21 | 34398114 | + | chr21: 34395128-34400245 | Island | OLIG2 |
| cg04186868 | 18 | 1.80% | chr12 | 57611144 | − | chr12: 57609976-57611168 | Island | NXPH4 |
| cg07060913 | 18 | 1.80% | chr16 | 86537142 | + | chr16: 86539118-86539486 | N_Shore | |
| cg09573795 | 18 | 1.80% | chr4 | 4863874 | + | chr4: 4864456-4864834 | N_Shore | MSX1 |
| cg19882268 | 18 | 1.80% | chr6 | 33245779 | − | chr6: 33244677-33245554 | S_Shore | B3GALT4 |
| cg20654074 | 18 | 1.80% | chr15 | 41223179 | + | chr15: 41217789-41223180 | Island | DLL4 |
| cg02503117 | 17 | 1.70% | chr16 | 86538424 | − | chr16: 86539118-86539486 | N_Shore | |
| cg08076158 | 17 | 1.70% | chr16 | 86539022 | − | chr16: 86539118-86539486 | N_Shore | |
| cg12626589 | 17 | 1.70% | chr6 | 32120783 | + | chr6: 32121829-32122529 | N_Shore | PPT2; PRRT1; PPT2 |
| cg13484546 | 15 | 1.50% | chr1 | 16084939 | − | chr1: 16085147-16085862 | N_Shore | FBLIM1 |
| cg14261472 | 15 | 1.50% | chr17 | 48637449 | + | chr17: 48636103-48639279 | Island | CACNA1G |
| cg14294793 | 15 | 1.50% | chr11 | 79150593 | + | chr11: 79148358-79152200 | Island | ODZ4 |
| cg15330117 | 15 | 1.50% | chr10 | 8096669 | − | chr10: 8091374-8098329 | Island | FLJ45983; GATA3 |
| cg17991695 | 15 | 1.50% | chr6 | 10882974 | + | chr6: 10882926-10883149 | Island | GCM2 |
| cg02694099 | 14 | 1.40% | chr15 | 71408914 | − | chr15: 71407656-71408498 | S_Shore | CT62 |
| cg11071401 | 14 | 1.40% | chr17 | 48637194 | + | chr17: 48636103-48639279 | Island | CACNA1G |
| cg15472071 | 14 | 1.40% | chr1 | 16085984 | + | chr1: 16085147-16085862 | S_Shore | FBLIM1 |
| cg08306084 | 13 | 1.30% | chr6 | 33248546 | − | chr6: 33244677-33245554 | S_Shelf | WDR46 |
| cg13882090 | 13 | 1.30% | chr6 | 33246094 | + | chr6: 33244677-33245554 | S_Shore | B3GALT4 |
| cg16662821 | 13 | 1.30% | chr8 | 41164679 | − | chr8: 41165852-41167140 | N_Shore | SFRP1 |
| cg19814946 | 13 | 1.30% | chr17 | 14205248 | − | chr17: 14204168-14207702 | Island | HS3ST3B1 |
| cg01546243 | 12 | 1.20% | chr14 | 61748019 | + | chr14: 61746804-61748141 | Island | TMEM30B |
| cg01626459 | 12 | 1.20% | chr6 | 56820778 | − | chr6: 56818873-56820308 | S_Shore | BEND6; DST |
| cg04216597 | 12 | 1.20% | chr17 | 48639836 | + | chr17: 48636103-48639279 | S_Shore | CACNA1G |
| cg07147364 | 12 | 1.20% | chr1 | 19970256 | − | chr1: 19970255-19971923 | Island | NBL1 |
| cg11303127 | 12 | 1.20% | chr12 | 49740807 | + | chr12: 49738680-49740841 | Island | DNAJC22 |
| cg11950383 | 12 | 1.20% | chr21 | 34400072 | − | chr21: 34395128-34400245 | Island | OLIG2 |
| cg16481280 | 12 | 1.20% | chr6 | 32120955 | + | chr6: 32121829-32122529 | N_Shore | PPT2; PRRT1 |
| cg19333963 | 12 | 1.20% | chr19 | 1467979 | + | chr19: 1465206-1471241 | Island | APC2 |
| cg21333861 | 12 | 1.20% | chr6 | 33244976 | − | chr6: 33244677-33245554 | Island | B3GALT4 |
| cg04641787 | 11 | 1.10% | chr10 | 8096154 | − | chr10: 8091374-8098329 | Island | FLJ45983; GATA3 |
| cg05620923 | 11 | 1.10% | chr19 | 1466647 | − | chr19: 1465206-1471241 | Island | APC2 |
| cg06018514 | 11 | 1.10% | chr15 | 41219741 | − | chr15: 41217789-41223180 | Island | |
| cg06133205 | 11 | 1.10% | chr13 | 50701960 | − | chr13: 50697984-50702286 | Island | |
| cg09255732 | 11 | 1.10% | chr1 | 32171050 | − | chr1: 32169537-32169869 | S_Shore | COL16A1 |
| cg09337254 | 11 | 1.10% | chr2 | 85640762 | + | chr2: 85640969-85641259 | N_Shore | |
| cg14040722 | 11 | 1.10% | chr20 | 37229509 | − | chr20: 37230523-37230742 | N_Shore | C20orf95 |
| cg15187550 | 11 | 1.10% | chr10 | 8096370 | − | chr10: 8091374-8098329 | Island | FLJ45983; GATA3 |
| cg16553500 | 11 | 1.10% | chr1 | 32169868 | + | chr1: 32169537-32169869 | Island | COL16A1 |
| cg18923740 | 11 | 1.10% | chr1 | 19971790 | − | chr1: 19970255-19971923 | Island | NBL1 |
| cg20682981 | 11 | 1.10% | chr17 | 1962627 | + | chr17: 1952919-1962328 | S_Shore | HIC1 |
| cg21249595 | 11 | 1.10% | chr6 | 30848811 | + | chr6: 30852102-30852676 | N_Shelf | |
| cg27390596 | 11 | 1.10% | chr17 | 48637858 | − | chr17: 48636103-48639279 | Island | CACNA1G |
| cg02962630 | 10 | 1.00% | chr15 | 41222776 | − | chr15: 41217789-41223180 | Island | DLL4 |
| cg10169241 | 10 | 1.00% | chr19 | 1467032 | − | chr19: 1465206-1471241 | Island | APC2 |
| cg12103626 | 10 | 1.00% | chr17 | 14204310 | − | chr17: 14204168-14207702 | Island | HS3ST3B1 |
| cg18932158 | 10 | 1.00% | chr6 | 33248279 | − | chr6: 33244677-33245554 | S_Shelf | WDR46 |
| cg19450714 | 10 | 1.00% | chr17 | 48637584 | + | chr17: 48636103-48639279 | Island | CACNA1G |
| cg01070078 | 9 | 0.90% | chr17 | 1958883 | − | chr17: 1952919-1962328 | Island | HIC1 |
| cg06774283 | 9 | 0.90% | chr17 | 26926076 | − | chr17: 26925742-26926512 | Island | SPAG5 |
| cg06814287 | 9 | 0.90% | chr6 | 32120584 | + | chr6: 32121829-32122529 | N_Shore | PPT2; PRRT1 |
| cg11145160 | 9 | 0.90% | chr3 | 170136278 | − | chr3: 170136242-170137886 | Island | CLDN11 |
| cg14130039 | 9 | 0.90% | chr6 | 32121225 | − | chr6: 32121829-32122529 | N_Shore | PPT2 |
| cg19036075 | 9 | 0.90% | chr15 | 74220295 | + | chr15: 74218696-74220373 | Island | LOXL1 |
| cg21538208 | 9 | 0.90% | chr4 | 4864488 | + | chr4: 4864456-4864834 | Island | MSX1 |
| cg22314314 | 9 | 0.90% | chr3 | 44802754 | − | chr3: 44802852-44803618 | N_Shore | KIF15; KIAA1143 |
| cg22322679 | 9 | 0.90% | chr6 | 33244178 | − | chr6: 33244677-33245554 | N_Shore | B3GALT4; RPS18 |
| cg23010452 | 9 | 0.90% | chr19 | 34013117 | + | chr19: 34012271-34012936 | S_Shore | PEPD |
| cg23047693 | 9 | 0.90% | chr12 | 57608606 | + | chr12: 57609976-57611168 | N_Shore | |
| cg00316759 | 8 | 0.80% | chr15 | 71407484 | − | chr15: 71407656-71408498 | N_Shore | CT62 |
| cg04209727 | 8 | 0.80% | chr18 | 30350441 | − | chr18: 30349690-30352302 | Island | KLHL14 |
| cg04856022 | 8 | 0.80% | chr6 | 32122955 | − | chr6: 32121829-32122529 | S_Shore | PPT2 |
| cg04877280 | 8 | 0.80% | chr6 | 32122738 | − | chr6: 32121829-32122529 | S_Shore | PPT2 |
| cg05945782 | 8 | 0.80% | chr17 | 1954986 | − | chr17: 1952919-1962328 | Island | MIR212 |
| cg26579986 | 8 | 0.80% | chr6 | 37504610 | − | chr6: 37503538-37504291 | S_Shore | |
| cg26704078 | 8 | 0.80% | chr18 | 24131115 | + | chr18: 24126780-24131138 | Island | KCTD1 |
| cg27147350 | 8 | 0.80% | chr6 | 33245881 | − | chr6: 33244677-33245554 | S_Shore | B3GALT4 |
| cg03740978 | 7 | 0.70% | chr18 | 24127875 | − | chr18: 24126780-24131138 | Island | KCTD1 |
| cg03839949 | 7 | 0.70% | chr3 | 128210541 | − | chr3: 128205495-128212274 | Island | GATA2 |
| cg04982951 | 7 | 0.70% | chr10 | 8096635 | + | chr10: 8091374-8098329 | Island | FLJ45983; GATA3 |
| cg05133205 | 7 | 0.70% | chr6 | 32121249 | − | chr6: 32121829-32122529 | N_Shore | PPT2 |
| cg08347183 | 7 | 0.70% | chr10 | 8096633 | + | chr10: 8091374-8098329 | Island | FLJ45983; GATA3 |
| cg10551329 | 7 | 0.70% | chr6 | 32120933 | + | chr6: 32121829-32122529 | N_Shore | PPT2; PRRT1 |
| cg16226644 | 7 | 0.70% | chr6 | 33246091 | − | chr6: 33244677-33245554 | S_Shore | B3GALT4 |
| cg20281962 | 7 | 0.70% | chr10 | 8089733 | − | chr10: 8091374-8098329 | N_Shore | |
| cg20914572 | 7 | 0.70% | chr6 | 32119874 | + | chr6: 32121829-32122529 | N_Shore | PRRT1; PPT2 |
| cg26366048 | 7 | 0.70% | chr6 | 56820386 | − | chr6: 56818873-56820308 | S_Shore | BEND6; DST |
| cg01312445 | 6 | 0.60% | chr16 | 86536684 | − | chr16: 86539118-86539486 | N_Shelf | |
| cg01993576 | 6 | 0.60% | chr6 | 44187674 | + | chr6: 44187186-44187400 | S_Shore | SLC29A1 |
| cg03995156 | 6 | 0.60% | chr6 | 32122864 | + | chr6: 32121829-32122529 | S_Shore | PPT2 |
| cg07555797 | 6 | 0.60% | chr14 | 61788314 | − | chr14: 61787880-61789467 | Island | PRKCH |
| cg09942293 | 6 | 0.60% | chr16 | 66957496 | − | chr16: 66958733-66959655 | N_Shore | RRAD |
| cg10372921 | 6 | 0.60% | chr15 | 74218733 | − | chr15: 74218696-74220373 | Island | LOXL1 |
| cg11941520 | 6 | 0.60% | chr6 | 32121522 | + | chr6: 32121829-32122529 | N_Shore | PPT2 |
| cg16396284 | 6 | 0.60% | chr6 | 33245537 | − | chr6: 33244677-33245554 | Island | B3GALT4 |
| cg16710894 | 6 | 0.60% | chr10 | 8092264 | − | chr10: 8091374-8098329 | Island | |
| cg20161179 | 6 | 0.60% | chr4 | 4863282 | + | chr4: 4864456-4864834 | N_Shore | MSX1 |
| cg24092179 | 6 | 0.60% | chr19 | 50931222 | − | chr19: 50931270-50931638 | N_Shore | SPIB |
| cg00552704 | 5 | 0.50% | chr6 | 32121420 | − | chr6: 32121829-32122529 | N_Shore | PPT2; PPT2 |
| cg05176991 | 5 | 0.50% | chr18 | 24128116 | + | chr18: 24126780-24131138 | Island | KCTD1 |
| cg06902929 | 5 | 0.50% | chr6 | 32123258 | + | chr6: 32121829-32122529 | S_Shore | PPT2; PPT2 |
| cg07273125 | 5 | 0.50% | chr16 | 68295692 | + | chr16: 68298012-68298979 | N_Shelf | |
| cg08483834 | 5 | 0.50% | chr6 | 33248239 | + | chr6: 33244677-33245554 | S_Shelf | WDR46 |
| cg08510658 | 5 | 0.50% | chr6 | 10882927 | − | chr6: 10882926-10883149 | Island | GCM2 |
| cg08890824 | 5 | 0.50% | chr16 | 66958786 | + | chr16: 66958733-66959655 | Island | RRAD |
| cg10094078 | 5 | 0.50% | chr19 | 1467925 | + | chr19: 1465206-1471241 | Island | APC2 |
| cg11215918 | 5 | 0.50% | chr21 | 34395699 | − | chr21: 34395128-34400245 | Island | |
| cg14167596 | 5 | 0.50% | chr4 | 4862910 | − | chr4: 4864456-4864834 | N_Shore | MSX1 |
| cg15852223 | 5 | 0.50% | chr10 | 8096372 | − | chr10: 8091374-8098329 | Island | FLJ45983; GATA3 |
| cg17639046 | 5 | 0.50% | chr17 | 14204027 | − | chr17: 14204168-14207702 | N_Shore | HS3ST3B1 |
| cg19951298 | 5 | 0.50% | chr6 | 10883054 | − | chr6: 10882926-10883149 | Island | GCM2 |
| cg20196291 | 5 | 0.50% | chr10 | 116164849 | − | chr10: 116163391-116164599 | S_Shore | AFAP1L2 |
| cg21973370 | 5 | 0.50% | chr17 | 1957919 | − | chr17: 1952919-1962328 | Island | HIC1 |
| cg22648949 | 5 | 0.50% | chr18 | 30351983 | + | chr18: 30349690-30352302 | Island | KLHL14 |
| cg26784201 | 5 | 0.50% | chr5 | 150050950 | − | chr5: 150051116-150052107 | N_Shore | MYOZ3 |
| cg00360474 | 4 | 0.40% | chr6 | 37504404 | + | chr6: 37503538-37504291 | S_Shore | |
| cg00930833 | 4 | 0.40% | chr8 | 41168264 | − | chr8: 41165852-41167140 | S_Shore | SFRP1 |
| cg01149449 | 4 | 0.40% | chr11 | 79150906 | + | chr11: 79148358-79152200 | Island | ODZ4 |
| cg02388150 | 4 | 0.40% | chr8 | 41165699 | − | chr8: 41165852-41167140 | N_Shore | SFRP1 |
| cg03718845 | 4 | 0.40% | chr2 | 85640001 | + | chr2: 85640969-85641259 | N_Shore | |
| cg03832440 | 4 | 0.40% | chr17 | 14207241 | + | chr17: 14204168-14207702 | Island | HS3ST3B1; MGC12916 |
| cg04414274 | 4 | 0.40% | chr17 | 1957866 | + | chr17: 1952919-1962328 | Island | HIC1 |
| cg06870728 | 4 | 0.40% | chr10 | 8095363 | − | chr10: 8091374-8098329 | Island | FLJ45983; GATA3 |
| cg07132710 | 4 | 0.40% | chr3 | 128202797 | − | chr3: 128205495-128212274 | N_Shelf | GATA2 |
| cg07306737 | 4 | 0.40% | chr6 | 33247141 | − | chr6: 33244677-33245554 | S_Shore | WDR46 |
| cg09857513 | 4 | 0.40% | chr7 | 120969044 | + | chr7: 120969587-120970743 | N_Shore | WNT16 |
| cg11014463 | 4 | 0.40% | chr6 | 56818479 | − | chr6: 56818873-56820308 | N_Shore | BEND6; DST |
| cg11626629 | 4 | 0.40% | chr6 | 33245460 | − | chr6: 33244677-33245554 | Island | B3GALT4 |
| cg12599673 | 4 | 0.40% | chr15 | 71408847 | − | chr15: 71407656-71408498 | S_Shore | CT62 |
| cg14293300 | 4 | 0.40% | chr21 | 34399361 | + | chr21: 34395128-34400245 | Island | OLIG2 |
| cg14904908 | 4 | 0.40% | chr8 | 41167660 | − | chr8: 41165852-41167140 | S_Shore | SFRP1 |
| cg15140798 | 4 | 0.40% | chr21 | 46782485 | − | chr21: 46785130-46785339 | N_Shelf | |
| cg15839448 | 4 | 0.40% | chr8 | 41166530 | − | chr8: 41165852-41167140 | Island | SFRP1 |
| cg17124583 | 4 | 0.40% | chr10 | 8097641 | − | chr10: 8091374-8098329 | Island | GATA3 |
| cg17764989 | 4 | 0.40% | chr16 | 86539121 | + | chr16: 86539118-86539486 | Island | |
| cg19156220 | 4 | 0.40% | chr6 | 33244752 | − | chr6: 33244677-33245554 | Island | B3GALT4 |
| cg22216643 | 4 | 0.40% | chr17 | 74704158 | − | chr17: 74706465-74707067 | N_Shelf | MXRA7 |
| cg23599559 | 4 | 0.40% | chr17 | 48637438 | − | chr17: 48636103-48639279 | Island | CACNA1G |
| cg24858591 | 4 | 0.40% | chr3 | 44803638 | − | chr3: 44802852-44803618 | S_Shore | KIAA1143; KIF15 |
| cg01160692 | 3 | 0.30% | chr17 | 1959620 | + | chr17: 1952919-1962328 | Island | HIC1 |
| cg01271812 | 3 | 0.30% | chr2 | 66671478 | − | chr2: 66672431-66673636 | N_Shore | MEIS1 |
| cg01626899 | 3 | 0.30% | chr17 | 26925852 | + | chr17: 26925742-26926512 | Island | SPAG5 |
| cg01684248 | 3 | 0.30% | chr16 | 86536239 | − | chr16: 86539118-86539486 | N_Shelf | |
| cg02980693 | 3 | 0.30% | chr3 | 128208970 | + | chr3: 128205495-128212274 | Island | GATA2 |
| cg03306486 | 3 | 0.30% | chr19 | 1467952 | + | chr19: 1465206-1471241 | Island | APC2 |
| cg06022942 | 3 | 0.30% | chr10 | 8095484 | + | chr10: 8091374-8098329 | Island | FLJ45983; GATA3 |
| cg06747432 | 3 | 0.30% | chr19 | 46916741 | + | chr19: 46916587-46916862 | Island | CCDC8 |
| cg06844968 | 3 | 0.30% | chr18 | 24131604 | − | chr18: 24126780-24131138 | S_Shore | KCTD1 |
| cg08438366 | 3 | 0.30% | chr20 | 37230612 | + | chr20: 37230523-37230742 | Island | C20orf95 |
| cg09042577 | 3 | 0.30% | chr11 | 119185584 | − | chr11: 119186947-119187894 | N_Shore | MCAM |
| cg09748975 | 3 | 0.30% | chr4 | 4864532 | + | chr4: 4864456-4864834 | Island | MSX1 |
| cg10464312 | 3 | 0.30% | chr2 | 66672688 | − | chr2: 66672431-66673636 | Island | MEIS1 |
| cg10633838 | 3 | 0.30% | chr6 | 33245359 | + | chr6: 33244677-33245554 | Island | B3GALT4 |
| cg13438549 | 3 | 0.30% | chr17 | 48633206 | + | chr17: 48636103-48639279 | N_Shelf | SPATA20 |
| cg15355859 | 3 | 0.30% | chr11 | 79149352 | − | chr11: 79148358-79152200 | Island | ODZ4 |
| cg15709766 | 3 | 0.30% | chr19 | 1466497 | − | chr19: 1465206-1471241 | Island | APC2 |
| cg17029019 | 3 | 0.30% | chr17 | 1959124 | − | chr17: 1952919-1962328 | Island | HIC1 |
| cg17891011 | 3 | 0.30% | chr10 | 8096152 | − | chr10: 8091374-8098329 | Island | FLJ45983; GATA3 |
| cg18774642 | 3 | 0.30% | chr18 | 30353699 | − | chr18: 30349690-30352302 | S_Shore | KLHL14 |
| cg19241689 | 3 | 0.30% | chr6 | 33245516 | − | chr6: 33244677-33245554 | Island | B3GALT4 |
| cg20706438 | 3 | 0.30% | chr2 | 74783005 | + | chr2: 74781494-74782685 | S_Shore | DOK1 |
| cg21068480 | 3 | 0.30% | chr2 | 85980500 | − | chr2: 85980499-85982198 | Island | ATOH8 |
| cg25520679 | 3 | 0.30% | chr17 | 1959121 | − | chr17: 1952919-1962328 | Island | HIC1 |
| cg26055446 | 3 | 0.30% | chr6 | 33245990 | + | chr6: 33244677-33245554 | S_Shore | B3GALT4 |
| cg00040007 | 2 | 0.20% | chr15 | 41222276 | − | chr15: 41217789-41223180 | Island | DLL4 |
| cg00927777 | 2 | 0.20% | chr17 | 1960199 | − | chr17: 1952919-1962328 | Island | HIC1 |
| cg01616215 | 2 | 0.20% | chr22 | 32340373 | − | chr22: 32339933-32341192 | Island | YWHAH; C22orf24 |
| cg01725608 | 2 | 0.20% | chr7 | 120969666 | − | chr7: 120969587-120970743 | Island | WNT16 |
| cg01785568 | 2 | 0.20% | chr4 | 4864833 | + | chr4: 4864456-4864834 | Island | MSX1 |
| cg01796075 | 2 | 0.20% | chr1 | 156878573 | − | chr1: 156877769-156878649 | Island | PEAR1 |
| cg02956248 | 2 | 0.20% | chr6 | 32120901 | − | chr6: 32121829-32122529 | N_Shore | PPT2; PRRT1; PPT2 |
| cg03814826 | 2 | 0.20% | chr22 | 32341378 | − | chr22: 32339933-32341192 | S_Shore | C22orf24; YWHAH |
| cg04203646 | 2 | 0.20% | chr19 | 1467008 | − | chr19: 1465206-1471241 | Island | APC2 |
| cg04751149 | 2 | 0.20% | chr2 | 66673449 | − | chr2: 66672431-66673636 | Island | MEIS1 |
| cg05003322 | 2 | 0.20% | chr1 | 32169706 | − | chr1: 32169537-32169869 | Island | COL16A1 |
| cg05871997 | 2 | 0.20% | chr6 | 56819623 | − | chr6: 56818873-56820308 | Island | BEND6; DST |
| cg06025456 | 2 | 0.20% | chr6 | 32120863 | + | chr6: 32121829-32122529 | N_Shore | PPT2; PRRT1; PPT2 |
| cg06283368 | 2 | 0.20% | chr15 | 74219669 | + | chr15: 74218696-74220373 | Island | LOXL1 |
| cg12881557 | 2 | 0.20% | chr18 | 24130633 | + | chr18: 24126780-24131138 | Island | KCTD1 |
| cg14250833 | 2 | 0.20% | chr6 | 10882240 | − | chr6: 10882926-10883149 | N_Shore | GCM2 |
| cg14914519 | 2 | 0.20% | chr17 | 14205882 | + | chr17: 14204168-14207702 | Island | HS3ST3B1; MGC12916 |
| cg16838838 | 2 | 0.20% | chr2 | 85641023 | + | chr2: 85640969-85641259 | Island | |
| cg16868298 | 2 | 0.20% | chr7 | 120969033 | + | chr7: 120969587-120970743 | N_Shore | WNT16 |
| cg17276021 | 2 | 0.20% | chr1 | 16084445 | + | chr1: 16085147-16085862 | N_Shore | FBLIM1 |
| cg17372269 | 2 | 0.20% | chr3 | 44802863 | − | chr3: 44802852-44803618 | Island | KIF15; KIAA1143 |
| cg18374181 | 2 | 0.20% | chr21 | 34401798 | − | chr21: 34395128-34400245 | S_Shore | |
| cg18729787 | 2 | 0.20% | chr6 | 33246307 | + | chr6: 33244677-33245554 | S_Shore | B3GALT4 |
| cg19884965 | 2 | 0.20% | chr11 | 79150305 | − | chr11: 79148358-79152200 | Island | ODZ4 |
| cg20138264 | 2 | 0.20% | chr17 | 48585640 | + | chr17: 48585385-48586167 | Island | MYCBPAP |
| cg20152539 | 2 | 0.20% | chr17 | 14206871 | + | chr17: 14204168-14207702 | Island | HS3ST3B1; MGC12916 |
| cg20180247 | 2 | 0.20% | chr6 | 10884140 | + | chr6: 10882926-10883149 | S_Shore | |
| cg20283670 | 2 | 0.20% | chr10 | 116162728 | − | chr10: 116163391-116164599 | N_Shore | AFAP1L2 |
| cg21435190 | 2 | 0.20% | chr3 | 128208037 | + | chr3: 128205495-128212274 | Island | GATA2 |
| cg23253569 | 2 | 0.20% | chr21 | 34398222 | + | chr21: 34395128-34400245 | Island | OLIG2 |
| cg24399924 | 2 | 0.20% | chr2 | 85980533 | − | chr2: 85980499-85982198 | Island | ATOH8 |
| cg24888989 | 2 | 0.20% | chr3 | 44803291 | − | chr3: 44802852-44803618 | Island | KIF15; KIF15; KIAA1143 |
| cg25075776 | 2 | 0.20% | chr6 | 30848828 | + | chr6: 30852102-30852676 | N_Shelf | |
| cg26418770 | 2 | 0.20% | chr17 | 14206886 | + | chr17: 14204168-14207702 | Island | HS3ST3B1; MGC12916 |
| cg26657382 | 2 | 0.20% | chr16 | 86538510 | − | chr16: 86539118-86539486 | N_Shore | |
| cg26977644 | 2 | 0.20% | chr11 | 79149294 | − | chr11: 79148358-79152200 | Island | ODZ4 |
| cg00183916 | 1 | 0.10% | chr17 | 14204936 | + | chr17: 14204168-14207702 | Island | HS3ST3B1 |
| cg00313401 | 1 | 0.10% | chr15 | 74219948 | + | chr15: 74218696-74220373 | Island | LOXL1 |
| cg00592510 | 1 | 0.10% | chr17 | 1957625 | + | chr17: 1952919-1962328 | Island | HIC1 |
| cg00702638 | 1 | 0.10% | chr3 | 44803293 | − | chr3: 44802852-44803618 | Island | KIF15; KIAA1143 |
| cg00739593 | 1 | 0.10% | chr10 | 116164714 | − | chr10: 116163391-116164599 | S_Shore | AFAP1L2 |
| cg00913604 | 1 | 0.10% | chr16 | 66958650 | − | chr16: 66958733-66959655 | N_Shore | RRAD |
| cg01404873 | 1 | 0.10% | chr13 | 50701050 | + | chr13: 50697984-50702286 | Island | DLEU2 |
| cg01807770 | 1 | 0.10% | chr4 | 79471305 | + | chr4: 79472806-79473177 | N_Shore | ANXA3 |
| cg02151609 | 1 | 0.10% | chr17 | 1957529 | − | chr17: 1952919-1962328 | Island | HIC1 |
| cg02242344 | 1 | 0.10% | chr2 | 85640943 | + | chr2: 85640969-85641259 | N_Shore | |
| cg02339682 | 1 | 0.10% | chr6 | 56819432 | − | chr6: 56818873-56820308 | Island | DST; BEND6 |
| cg02429905 | 1 | 0.10% | chr6 | 32119944 | − | chr6: 32121829-32122529 | N_Shore | PRRT1; PPT2 |
| cg02836487 | 1 | 0.10% | chr3 | 128206457 | − | chr3: 128205495-128212274 | Island | GATA2 |
| cg03133371 | 1 | 0.10% | chr8 | 41167673 | + | chr8: 41165852-41167140 | S_Shore | SFRP1 |
| cg03270204 | 1 | 0.10% | chr6 | 30851638 | − | chr6: 30852102-30852676 | N_Shore | DDR1 |
| cg03356734 | 1 | 0.10% | chr20 | 37230413 | + | chr20: 37230523-37230742 | N_Shore | C20orf95 |
| cg03365354 | 1 | 0.10% | chr11 | 119187391 | − | chr11: 119186947-119187894 | Island | MCAM |
| cg03434432 | 1 | 0.10% | chr6 | 32122393 | − | chr6: 32121829-32122529 | Island | PPT2 |
| cg03570994 | 1 | 0.10% | chr6 | 32121143 | + | chr6: 32121829-32122529 | N_Shore | PPT2; PRRT1 |
| cg03575666 | 1 | 0.10% | chr8 | 41168186 | + | chr8: 41165852-41167140 | S_Shore | SFRP1 |
| cg04105091 | 1 | 0.10% | chr6 | 32121355 | + | chr6: 32121829-32122529 | N_Shore | PPT2 |
| cg04436755 | 1 | 0.10% | chr15 | 74218767 | + | chr15: 74218696-74220373 | Island | LOXL1 |
| cg04852949 | 1 | 0.10% | chr1 | 32170929 | − | chr1: 32169537-32169869 | S_Shore | COL16A1 |
| cg04983516 | 1 | 0.10% | chr11 | 79151719 | + | chr11: 79148358-79152200 | Island | ODZ4 |
| cg05457563 | 1 | 0.10% | chr19 | 1467029 | − | chr19: 1465206-1471241 | Island | APC2 |
| cg05470554 | 1 | 0.10% | chr7 | 120969079 | − | chr7: 120969587-120970743 | N_Shore | WNT16 |
| cg05713782 | 1 | 0.10% | chr11 | 94706830 | − | chr11: 94706291-94707060 | Island | KDM4D; CWC15 |
| cg05946971 | 1 | 0.10% | chr22 | 32341328 | − | chr22: 32339933-32341192 | S_Shore | C22orf24; YWHAH |
| cg06065141 | 1 | 0.10% | chr17 | 1957161 | − | chr17: 1952919-1962328 | Island | HIC1 |
| cg06485671 | 1 | 0.10% | chr18 | 30350935 | − | chr18: 30349690-30352302 | Island | KLHL14 |
| cg06515159 | 1 | 0.10% | chr21 | 34400659 | + | chr21: 34395128-34400245 | S_Shore | OLIG2 |
| cg06642647 | 1 | 0.10% | chr6 | 30848807 | + | chr6: 30852102-30852676 | N_Shelf | |
| cg06892009 | 1 | 0.10% | chr11 | 79151611 | − | chr11: 79148358-79152200 | Island | ODZ4 |
| cg07137845 | 1 | 0.10% | chr3 | 170136485 | − | chr3: 170136242-170137886 | Island | CLDN11 |
| cg07265873 | 1 | 0.10% | chr6 | 30851940 | − | chr6: 30852102-30852676 | N_Shore | DDR1 |
| cg07348922 | 1 | 0.10% | chr6 | 33244990 | + | chr6: 33244677-33245554 | Island | B3GALT4 |
| cg07578663 | 1 | 0.10% | chr10 | 8096600 | + | chr10: 8091374-8098329 | Island | FLJ45983; GATA3; |
| cg08110052 | 1 | 0.10% | chr6 | 32125424 | + | chr6: 32121829-32122529 | S_Shelf | PPT2 |
| cg08509237 | 1 | 0.10% | chr6 | 32122065 | − | chr6: 32121829-32122529 | Island | PPT2 |
| cg08711175 | 1 | 0.10% | chr12 | 57614182 | − | chr12: 57609976-57611168 | S_Shelf | NXPH4 |
| cg09074260 | 1 | 0.10% | chr11 | 94707049 | + | chr11: 94706291-94707060 | Island | KDM4D; CWC15 |
| cg09172659 | 1 | 0.10% | chr17 | 14203711 | + | chr17: 14204168-14207702 | N_Shore | HS3ST3B1 |
| cg09410389 | 1 | 0.10% | chr8 | 41168205 | − | chr8: 41165852-41167140 | S_Shore | SFRP1 |
| cg09535924 | 1 | 0.10% | chr2 | 66671659 | + | chr2: 66672431-66673636 | N_Shore | MEIS1 |
| cg09570958 | 1 | 0.10% | chr17 | 14206774 | − | chr17: 14204168-14207702 | Island | HS3ST3B1; MGC12916 |
| cg09673208 | 1 | 0.10% | chr11 | 79151811 | + | chr11: 79148358-79152200 | Island | ODZ4 |
| cg09829319 | 1 | 0.10% | chr6 | 10882238 | − | chr6: 10882926-10883149 | N_Shore | GCM2 |
| cg10405604 | 1 | 0.10% | chr15 | 101390259 | + | chr15: 101389732-101390260 | Island | |
| cg10541674 | 1 | 0.10% | chr12 | 57610491 | − | chr12: 57609976-57611168 | Island | NXPH4 |
| cg10935762 | 1 | 0.10% | chr3 | 128202176 | + | chr3: 128205495-128212274 | N_Shelf | GATA2 |
| cg10948797 | 1 | 0.10% | chr17 | 1957607 | + | chr17: 1952919-1962328 | Island | HIC1 |
| cg11018337 | 1 | 0.10% | chr10 | 8095495 | + | chr10: 8091374-8098329 | Island | FLJ45983; GATA3 |
| cg11452354 | 1 | 0.10% | chr6 | 44187052 | + | chr6: 44187186-44187400 | N_Shore | SLC29A1 |
| cg11453400 | 1 | 0.10% | chr10 | 116165190 | − | chr10: 116163391-116164599 | S_Shore | AFAP1L2 |
| cg11471939 | 1 | 0.10% | chr15 | 72522966 | + | chr15: 72522131-72524238 | Island | PKM2 |
| cg12280317 | 1 | 0.10% | chr1 | 152008083 | + | chr1: 152008838-152009112 | N_Shore | S100A11 |
| cg12308216 | 1 | 0.10% | chr6 | 30853255 | + | chr6: 30852102-30852676 | S_Shore | DDR1 |
| cg13102294 | 1 | 0.10% | chr6 | 32121393 | − | chr6: 32121829-32122529 | N_Shore | PPT2 |
| cg13161961 | 1 | 0.10% | chr7 | 120970240 | + | chr7: 120969587-120970743 | Island | WNT16 |
| cg13333304 | 1 | 0.10% | chr3 | 170136200 | − | chr3: 170136242-170137886 | N_Shore | CLDN11 |
| cg13365340 | 1 | 0.10% | chr6 | 33245342 | + | chr6: 33244677-33245554 | Island | B3GALT4 |
| cg13431023 | 1 | 0.10% | chr10 | 8096220 | − | chr10: 8091374-8098329 | Island | FLJ45983; GATA3 |
| cg13524919 | 1 | 0.10% | chr21 | 34396506 | + | chr21: 34395128-34400245 | Island | |
| cg13543854 | 1 | 0.10% | chr10 | 8095477 | − | chr10: 8091374-8098329 | Island | FLJ45983; GATA3 |
| cg13793145 | 1 | 0.10% | chr6 | 44187109 | − | chr6: 44187186-44187400 | N_Shore | SLC29A1 |
| cg13915354 | 1 | 0.10% | chr17 | 1957671 | − | chr17: 1952919-1962328 | Island | HIC1 |
| cg13951527 | 1 | 0.10% | chr17 | 1957216 | − | chr17: 1952919-1962328 | Island | HIC1 |
| cg14435807 | 1 | 0.10% | chr15 | 74218780 | + | chr15: 74218696-74220373 | Island | LOXL1 |
| cg14448169 | 1 | 0.10% | chr7 | 120968904 | − | chr7: 120969587-120970743 | N_Shore | WNT16 |
| cg14775296 | 1 | 0.10% | chr2 | 66672841 | − | chr2: 66672431-66673636 | Island | MEIS1 |
| cg14843922 | 1 | 0.10% | chr21 | 34398849 | + | chr21: 34395128-34400245 | Island | OLIG2 |
| cg14950855 | 1 | 0.10% | chr12 | 49740781 | + | chr12: 49738680-49740841 | Island | DNAJC22 |
| cg15543281 | 1 | 0.10% | chr6 | 33245181 | + | chr6: 33244677-33245554 | Island | B3GALT4 |
| cg15657704 | 1 | 0.10% | chr10 | 116164955 | − | chr10: 116163391-116164599 | S_Shore | AFAP1L2 |
| cg15848031 | 1 | 0.10% | chr4 | 4864293 | + | chr4: 4864456-4864834 | N_Shore | MSX1 |
| cg15989091 | 1 | 0.10% | chr2 | 74780172 | + | chr2: 74781494-74782685 | N_Shore | LOXL3 |
| cg16004427 | 1 | 0.10% | chr1 | 16083101 | − | chr1: 16085147-16085862 | N_Shelf | |
| cg16079541 | 1 | 0.10% | chr6 | 30848846 | + | chr6: 30852102-30852676 | N_Shelf | |
| cg16437908 | 1 | 0.10% | chr2 | 85640810 | − | chr2: 85640969-85641259 | N_Shore | |
| cg16477774 | 1 | 0.10% | chr11 | 65325249 | − | chr11: 65325081-65326209 | Island | LTBP3 |
| cg16713743 | 1 | 0.10% | chr21 | 34397135 | + | chr21: 34395128-34400245 | Island | OLIG2 |
| cg18729886 | 1 | 0.10% | chr14 | 61788339 | − | chr14: 61787880-61789467 | Island | PRKCH |
| cg19873719 | 1 | 0.10% | chr6 | 33247107 | + | chr6: 33244677-33245554 | S_Shore | WDR46 |
| cg20457147 | 1 | 0.10% | chr14 | 61787823 | − | chr14: 61787880-61789467 | N_Shore | PRKCH |
| cg20459712 | 1 | 0.10% | chr6 | 56815929 | + | chr6: 56818873-56820308 | N_Shelf | DST |
| cg20731875 | 1 | 0.10% | chr17 | 14207701 | + | chr17: 14204168-14207702 | Island | HS3ST3B1; MGC12916 |
| cg21415424 | 1 | 0.10% | chr6 | 37503074 | + | chr6: 37503538-37504291 | N_Shore | |
| cg22609784 | 1 | 0.10% | chr4 | 4863678 | + | chr4: 4864456-4864834 | N_Shore | MSX1 |
| cg22745102 | 1 | 0.10% | chr19 | 50931616 | + | chr19: 50931270-50931638 | Island | SPIB |
| cg22913903 | 1 | 0.10% | chr12 | 49740968 | − | chr12: 49738680-49740841 | S_Shore | DNAJC22 |
| cg22931738 | 1 | 0.10% | chr3 | 128206823 | + | chr3: 128205495-128212274 | Island | GATA2 |
| cg23305408 | 1 | 0.10% | chr1 | 32169701 | − | chr1: 32169537-32169869 | Island | COL16A1 |
| cg23519308 | 1 | 0.10% | chr19 | 34012901 | − | chr19: 34012271-34012936 | Island | PEPD |
| cg23621097 | 1 | 0.10% | chr17 | 1962236 | + | chr17: 1952919-1962328 | Island | HIC1; HIC1 |
| cg23950233 | 1 | 0.10% | chr6 | 33245739 | − | chr6: 33244677-33245554 | S_Shore | B3GALT4 |
| cg24506025 | 1 | 0.10% | chr11 | 94706874 | + | chr11: 94706291-94707060 | Island | KDM4D; CWC15 |
| cg25161092 | 1 | 0.10% | chr2 | 85638535 | + | chr2: 85640969-85641259 | N_Shelf | CAPG |
| cg25484790 | 1 | 0.10% | chr11 | 119185671 | − | chr11: 119186947-119187894 | N_Shore | MCAM |
| cg26709950 | 1 | 0.10% | chr16 | 66959235 | + | chr16: 66958733-66959655 | Island | RRAD |
| cg27038439 | 1 | 0.10% | chr4 | 4864320 | − | chr4: 4864456-4864834 | N_Shore | MSX1 |
| cg27070869 | 1 | 0.10% | chr6 | 32122779 | − | chr6: 32121829-32122529 | S_Shore | PPT2 |
| cg27357571 | 1 | 0.10% | chr21 | 34398226 | + | chr21: 34395128-34400245 | Island | OLIG2 |

TABLE 4
List of CpGs and annotation for the methylated CpGs reoccurring in at least 10% of the minimal LASSO models.

| | No of | | | | | | | |
|---|---|---|---|---|---|---|---|---|
| CpG | times used | Percentage | chr | pos | strand | Islands_Name | Relation_to_Island | UCSC_RefGene_Name |
| cg01811187 | 767 | 76.70% | chr17 | 48637445 | + | chr17: 48636103-48639279 | Island | CACNA1G |
| cg17078427 | 703 | 70.30% | chr3 | 170137552 | − | chr3: 170136242-170137886 | Island | CLDN11 |
| cg16547027 | 462 | 46.20% | chr18 | 24127588 | − | chr18: 24126780-24131138 | Island | KCTD1 |
| cg19596468 | 458 | 45.80% | chr4 | 4864110 | + | chr4: 4864456-4864834 | N_Shore | MSX1 |
| cg14309111 | 430 | 43.00% | chr11 | 79150411 | + | chr11: 79148358-79152200 | Island | ODZ4 |
| cg17603502 | 415 | 41.50% | chr17 | 14204056 | − | chr17: 14204168-14207702 | N_Shore | HS3ST3B1 |
| cg08133931 | 384 | 38.40% | chr17 | 48636626 | + | chr17: 48636103-48639279 | Island | |
| cg18599069 | 342 | 34.20% | chr10 | 8096991 | + | chr10: 8091374-8098329 | Island | GATA3 |
| cg24840099 | 239 | 23.90% | chr4 | 4864430 | + | chr4: 4864456-4864834 | N_Shore | MSX1 |
| cg09529433 | 220 | 22.00% | chr17 | 48637255 | + | chr17: 48636103-48639279 | Island | CACNA1G |
| cg10096645 | 220 | 22.00% | chr18 | 24130851 | + | chr18: 24126780-24131138 | Island | KCTD1 |
| cg06108383 | 211 | 21.10% | chr6 | 32120899 | − | chr6: 32121829-32122529 | N_Shore | PPT2; PRRT1 |
| cg03884082 | 172 | 17.20% | chr1 | 19971709 | + | chr1: 19970255-19971923 | Island | NBL1 |
| cg01065003 | 171 | 17.10% | chr18 | 24130839 | − | chr18: 24126780-24131138 | Island | KCTD1 |
| cg22647713 | 168 | 16.80% | chr10 | 8095697 | − | chr10: 8091374-8098329 | Island | FLJ45983; GATA3 |
| cg20449692 | 162 | 16.20% | chr3 | 170136920 | − | chr3: 170136242-170137886 | Island | CLDN11 |
| cg07136023 | 150 | 15.00% | chr16 | 86537316 | − | chr16: 86539118-86539486 | N_Shore | |
| cg20811659 | 136 | 13.60% | chr17 | 48637730 | − | chr17: 48636103-48639279 | Island | CACNA1G |
| cg20048434 | 132 | 13.20% | chr10 | 116163160 | − | chr10: 116163391-116164599 | N_Shore | AFAP1L2 |
| cg06546607 | 127 | 12.70% | chr19 | 34013019 | + | chr19: 34012271-34012936 | S_Shore | PEPD |
| cg00403498 | 127 | 12.70% | chr6 | 32119923 | − | chr6: 32121829-32122529 | N_Shore | PRRT1; PPT2 |
| cg20891301 | 119 | 11.90% | chr4 | 4864711 | − | chr4: 4864456-4864834 | Island | MSX1 |
| cg17416730 | 116 | 11.60% | chr6 | 33245541 | − | chr6: 33244677-33245554 | Island | B3GALT4 |
| cg01724566 | 113 | 11.30% | chr17 | 26926132 | + | chr17: 26925742-26926512 | Island | SPAG5 |
| cg16501308 | 112 | 11.20% | chr18 | 30350221 | − | chr18: 30349690-30352302 | Island | KLHL14 |
| cg06230736 | 109 | 10.90% | chr10 | 8096650 | + | chr10: 8091374-8098329 | Island | FLJ45983; GATA3 |
| cg03199651 | 105 | 10.50% | chr4 | 4862770 | − | chr4: 4864456-4864834 | N_Shore | MSX1 |
| cg06329022 | 103 | 10.30% | chr17 | 26926511 | + | chr17: 26925742-26926512 | Island | SPAG5 |
| cg13879776 | 102 | 10.20% | chr3 | 170136263 | − | chr3: 170136242-170137886 | Island | CLDN11 |

TABLE 5
the number of CpGs reoccurring or used in the minimal models

| Used equal or more than | Nr CpGs |
|---|---|
| 1% | 169 |
| 2% | 119 |
| 3% | 93 |
| 4% | 70 |
| 5% | 61 |
| 6% | 52 |
| 7% | 41 |
| 8% | 36 |
| 9% | 33 |
| 10% | 29 |
| 20% | 12 |
| 30% | 8 |
| 40% | 6 |
| 50% | 2 |
| 60% | 2 |
| 70% | 2 |
| 80% | 0 |
| 90% | 0 |
| 100% | 0 |

### DISCUSSION

In the multi-cohort epigenome-wide study, it was demonstrated that cold ischemia occurring during kidney transplantation induced DNA hypermethylation of allografts through reduced TET DNA-demethylation activity. The observed hypermethylation changes remained stable for months after transplantation, downregulated expression of associated genes and preferentially affected genes involved in suppression of kidney fibrosis and injury. Importantly, the resultant methylation signature could predict future chronic allograft injury, and this with a predictive power that is superior compared to a combination of clinical variables routinely monitored in clinical practice. In some CpGs, the observed DNA hypermethylation was quite substantial, with changes mounting up to 2.6% for each additional hour of cold ischemia time. With cold ischemia for some transplants lasting over 24 hours, the cumulative effect on the DNA methylome thus could become quite impactful. DNA hypermethylation was moreover observed in different cohorts involving biopsies obtained at different time points (e.g., pre-implantation versus post-reperfusion), thereby underscoring the robustness of the findings. Several of the observations also suggest that DNA hypermethylation causally contributes to chronic allograft injury. For instance, ischemia-induced hypermethylation was observed predominantly near genes involved in the ‘negative’ regulation of fibrosis and cell death. Hypermethylation silenced expression of affected genes and thereby thus triggers allograft injury. The ischemia-induced hypermethylation was also evident up to one year after transplantation, which is a prerequisite for DNA methylation to induce long-term histological changes in kidney transplants.

Notably, the concept of DNA hypermethylation being causal for chronic allograft injury also induced a shift in the pathophysiology underlying ischemia-induced chronic allograft injury. Hitherto, chronic allograft injury has mainly been considered to be driven indirectly by a host immune response to acute injury⁴. These data support a more direct and lasting effect of ischemia on graft fibrosis, and suggest that inhibitors of DNA methylation or inducers of TET expression represent therapeutic agents to prevent chronic allograft injury. Indeed, DNA methylation changes are generally considered to be reversible, and DNA methylation inhibitors are already approved for the treatment of hematological malignancies¹⁵.

These findings also reveal important biomarker potential. Indeed, the presented method allow to reliably predict CAI 1 year after transplantation by assessing methylation at the time of transplantation in those CpG islands becoming consistently hypermethylated upon ischemia. In an independent replication cohort, the tertile of patients with the highest methylation risk score exhibited a 9-fold increased risk of developing allograft injury, relative to patients with the lowest risk, in the lowest tertile. Currently, the risk of developing chronic allograft injury is estimated based on clinical risk factors, such as donor age and ischemia time, but in a head-to-head comparison our methylation risk score outperformed the combined predictive effect of these baseline clinical variables. Notably, the methylation risk score presented here, which is a direct consequence of kidney ischemia, predicted chronic allograft injury independently of the duration of ischemia, as measured during transplantation. This suggests that methylation captures the different susceptibility of kidneys to ischemia.

Mechanistically, these findings build on the observations in solid tumors, in which reduced TET DNA-demethylation activity led to DNA hypermethylation of gene promoters and enhancers⁸. TET enzymes are Fe²⁺- and α-ketoglutarate dependent dioxygenases that oxidize 5mC to 5hmC¹⁷, which is then further oxidized to other demethylation intermediates and subsequently replaced by an unmodified cytosine, leading to DNA demethylation¹⁸. In line with these findings, DNA hypermethylation was also enriched in kidney allografts subjected to cold ischemia in regions known to be TET binding sites, i.e., gene promoter and enhancer regions⁷. Furthermore, each hypermethylation event was mirrored by an inverse change in 5hmC, indicating that DNA hypermethylation occurs through reduced TET activity. Although the underlying mechanisms in transplanted kidneys thus seems to be akin to those operating in tumors, the observations are quite surprising. Indeed, in transplanted kidneys oxygen levels are lower than in tumours (0.1% versus 0.3-0.5%), but ischemia time is much shorter (on average 24 hours during transplantation versus months to even years in tumors). Furthermore, cancer cells are highly proliferative and can select for epigenetic changes conferring a survival benefit. In contrast, kidneys are characterized by low levels of cell proliferation, which reduces the potential for stabilisation of epigenetic changes through cellular selection. Interestingly, the functional implications of these findings could be translated to other fields of medicine. Indeed, besides obvious implications in other transplant settings, they may be of relevance for other ischemic diseases, for which it would be less straightforward to demonstrate similar mechanisms. Performing paired biopsies in patients is indeed nearly impossible in other ischemic diseases, such as stroke or myocardial infarction, and also the correlation of epigenetic changes with ischemia time would be challenging, as the exact onset of ischemia is almost impossible to determine in these pathologies.

In conclusion, a novel, epigenetic mechanism is described here that links ischemia at the time of kidney transplantation with progressive chronic allograft injury after transplantation, disclosing the essential event of DNA hypermethylation on a number of specific CpGs located in several CpG islands. Since DNA methylation is generally considered to be reversible, these results have therapeutic applications for the prevention of chronic allograft injury, a disease that is currently lacking therapeutic options.

### Methods

### Study Design and Patients

We subjected 3 different cohorts of kidney transplants to genome-wide DNA methylation profiling: a longitudinal cohort of 13×2 paired procurement (pre-ischemia) and post-reperfusion (post-ischemia) kidney transplant biopsies, with an additional biopsy 3 or 12 months after transplantation in a subgroup (n=2×5); a second pre-implantation cohort of biopsies obtained immediately prior to implantation (n=82); a third cohort of post-reperfusion biopsies (n=46; post-reperfusion cohort). We additionally collected 10 post-reperfusion biopsies, 5 from living donor kidney transplantations versus 5 from deceased donor transplantations with long cold ischemia times to validate DNA hydroxymethylation changes through LC-MS. Machine-perfused kidneys were excluded from all cohorts. All transplant recipients gave written informed consent and the study was approved by the Ethical Review Board of the University Hospitals Leuven (S53364).

### Epigenome-Wide Methylation Profiling

Genomic DNA was extracted from all biopsies using Allprep DNA/RNA/miRNA Universal kit (Qiagen, Hilden, Germany). For genome-wide methylation analysis, DNA was bisulphite converted using EZ DNA Methylation kit (Zymo Research, Irvine, Calif., USA) and subsequently probed for DNA methylation levels using the Illumina EPIC array (for the longitudinal and pre-implantation cohort) or the 450K array²⁴ (for the post-reperfusion cohort). TET-assisted bisulphite conversion was used for hydroxymethylation analysis, as described.⁸ Quality control consisted of: removal of probes for which any sample did not pass a 0.01 detection P value threshold, bead cut-off of 0.05, and removal of probes on sex chromosomes. Probe annotation was performed using Minfi¹⁹.

### Gene Expression Profiling

RT-PCR was performed using OpenArray technology, a real-time PCR-based solution for high-throughput gene expression analysis (Quantstudio 12K Flex Real-Time PCR system, Thermofisher Scientific, Ghent, Belgium) for 70 transcripts that corresponded to the protein-coding genes associated with the 66 CpG islands that were hypermethylated upon ischemia at FDR<0.05 in both cohorts, and for the DNA methylation modifiers TET1, TET2, TET3, DNMT1, DNMT3A, DNMT3B, DNMT3L. Five housekeeping genes (B2M, 18S, TBP, RPL13A, YWHAZ) were selected according to the literature, of which 18S, TBP and YWHAZ were considered adequate based on the gene expression changes pre- versus post-ischemia. Five of 70 transcripts failed.

### Statistical Analyses

Statistical analyses were performed using RStudio (version 0.99). Raw methylation data were normalised using BMIQ and batch corrected using Combat, with the ChAMP pipeline²⁰. Methylation levels (beta-values) were logarithmically transformed to M-values for all statistical tests, unless stated otherwise. Results are presented as P values and FDR values using the Benjamini and Hochberg method. LC-MS to determine unmethylated C, 5mC and 5hmC concentrations in the transplant genome was performed as described.⁸ In the longitudinal cohort, we compared DNA methylation and hydroxymethylation levels pre- versus post-ischemia overall using Wilcoxon signed-rank and paired t-tests respectively, and subsequently at CpG-site level. In the pre-implantation cohort, we examined the effect of cold ischemia time expressed as a continuous variable (in hours) on DNA methylation for all CpGs using linear regression adjusted for donor age and gender, since age and gender are major determinants of the DNA methylome. In addition, individual CpGs were grouped according to their associated CpG island (including shores and shelves) and similar analyses were performed for CpG islands: in the longitudinal cohort by paired t-tests per island and in the pre-implantation cohort using a linear mixed model, adjusted for donor age and gender, and with transplant identifier as a random effect. To evaluate locus-specifically whether changes in 5mC are mirrored by inverse changes in 5hmC in the longitudinal cohort, 5mC levels for this particular analysis were estimated by subtracting 5hmC from 5mC, as described previously⁸, since 5mC and 5hmC are both measured as 5mC after bisulphite conversion.

Hyper- versus hypomethylation events were compared using binomial tests. Overlap between cohorts was investigated by χ² analysis. We annotated ischemia-hypermethylated probes in both cohorts to their chromatin state using chromHMM data annotated for human fetal kidney²¹. Pathway analysis was performed using DAVID, gene ontology enrichment using topGO in R.

Gene expression in each post-ischemia sample was calculated relative to the expression of the reference pre-ischemia sample, using the ΔΔCt method with log 2 transformation.

Ischemia-induced hypermethylation was correlated with the CADI score in protocol-specified allograft biopsies obtained at 3 months and 1 year after transplantation. Analyses were done unadjusted and adjusted for donor age (the major determinant of chronic injury)²² and donor gender (which influences DNA methylation), and in a separate analysis also for cold and warm ischemia time.

Methylation values are usually expressed as “beta values”. Beta values (β) are the estimate of methylation level using the ratio of intensities between methylated and unmethylated alleles. β values range between 0 and 1, with β=0 being unmethylated and β=1 being fully methylated.

A methylation risk score (MRS) was developed to predict chronic injury (CADI-score>2) at 1 year after transplantation. For this, we first selected all 66 CpG islands that were hypermethylated due to transplantation-induced ischemia in two cohorts (i.e., the paired biopsy cohort and the pre-implantation biopsy cohort). These 66 CpG islands contained 1,634 CpGs. From these, we selected all 1,238 CpGs that are also measured using 450K arrays (to allow our 850K array-based methylation data to be replicated in the post-implantation biopsy cohort, which was profiled using 450K Illumina arrays only). Then, we correlated methylation (beta) values from each of the 1,238 CpGs located in these 66 CpG islands with chronic injury (CADI>2) in the pre-implantation cohort. For this, a logistic regression model containing each of the 1238 CpGs was fit using ridge regression to penalize the coefficient estimates. Ridge regression was chosen because it is better suited for logistic models with many input variables and also because it can handle input variables that are dependent from each other (which is necessary here because CpGs that belong to a CpG island are often co-regulated at the methylation level). This resulted in a logistic model, in which a coefficient was assigned to each individual CpG. Next, the methylation risk score was defined as the sum of methylation (beta) values at each CpG in 66 ischemia-hypermethylated CpG islands, weighted by marker-specific effect sizes (i.e., multiplied by the coefficient obtained for this CpG in the logistic regression model). The DNA methylation risk score was correlated to allograft function at 1 year after transplantation using the estimated glomerular filtration rate (eGFR) calculated by the MDRD formula²³.

The formula for calculating the methylation risk score (MRS) as outlined above is: MRS=intercept+c₁β₁+c₂β₂+c₃β₃+ . . . c₁₂₃₈β₁₂₃₈. The methylation risk score, consisting of the same coefficients that were determined in the pre-implantation discovery cohort (c₁, c₂, c₃, c₄, . . . , c₀₂₃₈) was subsequently validated in the post-reperfusion cohort.

The MRS can be calculated for n methylation markers wherein n is the actual number of methylation markers. For instance, n=4, 5, 6, 7, 8, 9, 10, 11, 12, 13, 14, 15, 16, 17, 18, 19, 20, 21, 22, 23, 24, 25, 26, 27, 28, 29 or more. In the context of the invention, the maximum value of n is 1238.

The values of the marker-specific coefficients and intercept obtained with the above described regression method are listed in Table 6. As these values were determined based on the pre-implantation discovery cohort and were validated independently in the post-reperfusion cohort, these can be considered to be relatively stable. Obviously, however, when running the same regression method on smaller or larger cohorts, this may result in variation of these marker-specific coefficients and intercept values.

TABLE 6
CpG-specific coefficients and the intercept value determined based on the
pre-implantation cohort, as validated in the post-reperfusion cohort.
[the values labeled with a “#” represent coefficients from the 29 CpGs listed in Table 4].

| | CpG | coefficient | | CpG | coefficient | | CpG | coefficient |
|---|---|---|---|---|---|---|---|---|
| | cg02980693 | 0.006500418 | | cg06796779 | 0.000463153 | | cg01751470 | 0.002485924 |
| | cg11968091 | 0.004094009 | | cg10739556 | −0.000322577 | | cg07265873 | 0.008782323 |
| | cg07159871 | −0.006325367 | | cg16650717 | 0.004934864 | | cg08076158 | 0.00458554 |
| | cg07603357 | −0.015485068 | | cg25449542 | 0.003112226 | | cg21446725 | −0.000534192 |
| | cg00266715 | −0.003922459 | | cg16674492 | 0.003307716 | | cg25179358 | 0.011634914 |
| | cg13934606 | −0.000531151 | | cg27038439 | 0.004502838 | | cg24399924 | 0.002794718 |
| | cg03921149 | −0.009256188 | | cg15949805 | −0.004539222 | | cg15225532 | −0.002481039 |
| | cg12700033 | −8.65E−05 | | cg24398479 | 0.001643149 | | cg15852223 | 0.010496735 |
| | cg22208012 | 0.006750482 | | cg12549595 | 0.000548524 | | cg24462596 | 0.000108676 |
| | cg23253569 | 0.005876635 | | cg02756676 | −0.004470999 | | cg05218311 | 0.005901937 |
| | cg06206902 | 0.000570349 | | cg27070869 | 0.002136454 | | cg14775296 | 0.00592916 |
| | cg22903655 | −0.005869433 | | cg01616215 | −0.001442367 | | cg13814485 | 0.003265676 |
| | cg11941520 | 0.005566437 | | cg11287851 | −0.001120405 | | cg14294793 | 0.004147921 |
| | cg27134827 | 0.000876568 | | cg16384862 | −0.000745074 | | cg06892009 | 0.005835542 |
| | cg05713782 | 0.008669041 | | cg17622922 | 0.00445377 | | cg03513246 | 0.007696404 |
| | cg27373972 | 0.001038863 | | cg23002761 | 0.002292629 | | cg17969084 | 0.000431397 |
| | cg27254482 | 0.008953678 | | cg12255698 | −0.00822135 | | cg01626899 | 0.005110751 |
| | cg06490988 | 0.006867259 | | cg13793145 | 0.002000856 | | cg19333963 | 0.005793853 |
| | cg13161961 | 0.00765146 | | cg16580935 | 0.019711056 | | cg10169241 | 0.002855451 |
| | cg21517947 | 0.004098578 | | cg26690075 | −0.005933343 | | cg00296182 | 0.004827306 |
| | cg05634149 | −0.002244196 | | cg06803850 | 0.004809573 | | cg27098900 | 0.001145787 |
| | cg24173182 | 0.003577569 | | cg26864130 | 0.005606665 | | cg12447069 | 0.001882452 |
| | cg15187550 | 0.006381623 | | cg09662694 | 0.00336717 | | cg04946603 | 0.003856932 |
| | cg18465199 | −0.001898096 | | cg05356738 | 0.010392332 | | cg04880618 | 0.001689956 |
| | cg09181339 | 0.008344582 | | cg19058189 | 0.005732522 | | cg06833110 | 0.006241838 |
| | cg21333861 | 0.001623111 | | cg11950383 | 0.015828024 | | cg06747432 | 0.002613791 |
| | cg11018337 | 0.002149647 | | cg24168641 | 0.006561193 | | cg07578663 | 0.002761419 |
| | cg01603146 | −0.000202749 | | cg23085676 | −0.009405551 | | cg25440818 | −0.003566128 |
| | cg02858594 | −3.00E−05 | | cg22214565 | −0.001125975 | | cg18932158 | 0.018027897 |
| | cg16838838 | 0.00299326 | | cg13181251 | 0.001185188 | | cg21387418 | 0.000773631 |
| | cg03434432 | 0.003254039 | | cg19764325 | −0.004477126 | | cg25738958 | −0.000464941 |
| | cg23737112 | 0.002705541 | | cg20467136 | 0.001338666 | | cg09599399 | 0.004887701 |
| | cg22931738 | 0.010068541 | | cg22878489 | 0.008040152 | | cg23768829 | −0.009975384 |
| | cg15839448 | 0.001985557 | | cg00969047 | −0.002537574 | | cg11071401 | 0.006674923 |
| | cg27357571 | 0.010145275 | | cg08057899 | 0.000915738 | | cg09748975 | 0.003177171 |
| | cg00012992 | 0.004378612 | | cg27542609 | 0.002627206 | | cg16477774 | 0.000490824 |
| | cg03724763 | −0.000291894 | | cg14950855 | 0.00180435 | | cg20792208 | −0.002883434 |
| | cg02342533 | −0.0071646 | | cg22571038 | 0.000932341 | | cg12883279 | 0.004705064 |
| | cg11452354 | 0.002486222 | | cg15657704 | 0.000965035 | | cg12930553 | 0.000481914 |
| | cg24280645 | −0.002935237 | | cg04528217 | −0.000283699 | | cg04767934 | 0.012671532 |
| | cg03648711 | 0.001597292 | | cg15233292 | 0.002840888 | | cg04890495 | 0.008212577 |
| | cg22513356 | 0.004468195 | | cg10383028 | 0.000767768 | | cg20596273 | −0.000593797 |
| | cg12573516 | 0.003049592 | | cg19816667 | −0.012601649 | | cg07442105 | −0.000674803 |
| | cg03306486 | 0.005708827 | | cg08469255 | 0.000869046 | | cg21654383 | 0.001122221 |
| | cg22242148 | 0.004689332 | | cg22122410 | −0.001548547 | | cg20152539 | 0.006537006 |
| | cg23160336 | −0.005066827 | | cg02573468 | 0.000576064 | | cg13579562 | 0.010376445 |
| | cg06273010 | 0.002055011 | | cg17029019 | 0.002628919 | | cg03421485 | −0.003000522 |
| | cg16011800 | −0.003159271 | | cg20954975 | −0.007535492 | | cg27382861 | −6.54E−05 |
| | cg14824386 | 0.008119209 | | cg26556926 | −0.000856215 | | cg19759251 | −0.000446087 |
| | cg21854952 | 0.002440653 | | cg25893992 | 0.003014049 | | cg01495122 | −0.000668356 |
| | cg13934406 | 0.00612937 | | cg04631281 | 0.00049553 | | cg10372921 | 0.00423855 |
| | cg17566118 | 0.007551958 | | cg14910368 | −0.001347642 | | cg16048942 | 0.004112181 |
| | cg18337803 | −0.000322082 | | cg01070985 | 0.007097817 | # | cg20449692 | 0.01039621 |
| | cg15299832 | −0.00061107 | | cg11303127 | 0.006761406 | | cg18318818 | 0.003713611 |
| | cg07348922 | 0.001459112 | | cg00815093 | 0.003567996 | | cg01224891 | 0.006516871 |
| | cg08045906 | 0.001686856 | | cg24607783 | −0.004983599 | | cg20281962 | 0.003466691 |
| | cg18454685 | 0.006450347 | | cg05620923 | 0.006627089 | | cg07240554 | −0.003943083 |
| | cg12246510 | −0.002779231 | | cg06462684 | 0.006091822 | # | cg07136023 | 0.01287055 |
| | cg20382774 | 0.0018393 | | cg15170634 | 0.00183145 | | cg15310583 | −0.002454585 |
| | cg06753439 | 0.009261373 | | cg07939626 | −0.001558861 | | cg13065834 | −0.001636049 |
| | cg24319902 | 0.000203523 | | cg18051461 | 0.003364814 | | cg20664636 | 0.003550119 |
| | cg24092179 | 0.00803102 | | cg11144056 | 0.001110683 | | cg22934970 | 0.00414965 |
| | cg02409108 | 0.001152966 | | cg12154045 | 0.009385742 | | cg02300764 | −0.003026841 |
| | cg14965968 | 0.006903027 | | cg23614229 | 0.001721088 | | cg07159490 | 0.006342173 |
| | cg07187855 | 0.000234235 | | cg13365340 | 0.002000315 | | cg23359665 | 0.004633137 |
| | cg09633973 | −0.000605051 | | cg25365746 | 0.002260995 | | cg27403810 | 0.000489267 |
| | cg05445638 | −0.002357865 | | cg18065337 | −0.003241732 | | cg10010386 | 0.002287058 |
| | cg03970849 | 0.011656144 | | cg03740978 | 0.00277028 | | cg17276021 | 0.005645876 |
| | cg14250833 | 0.006525128 | | cg00847029 | 0.005718902 | | cg05133205 | 0.002796899 |
| | cg18049167 | 0.001884743 | | cg26128977 | −0.012546923 | | cg14531560 | 0.002196359 |
| | cg14610962 | −0.00398979 | | cg19664267 | 0.001158411 | | cg16766889 | −0.005611265 |
| | cg10555159 | 0.004677015 | | cg13389502 | 0.000664699 | | cg21556389 | 0.005490237 |
| | cg17171962 | −0.001888264 | | cg26169408 | 0.003898202 | | cg15330117 | 0.008570295 |
| | cg16396284 | 0.009130069 | | cg06025456 | 0.00318856 | | cg15007959 | −0.000971312 |
| | cg05470554 | −0.001406791 | | cg15267232 | 0.004882155 | | cg12048339 | −0.004496756 |
| | cg22151941 | 0.005769607 | | cg19385386 | 0.007015264 | | cg14985989 | 0.004867004 |
| | cg09389280 | 0.009664574 | | cg14771810 | 0.003164545 | | cg24888989 | 0.00324759 |
| | cg05871997 | 0.01124777 | | cg24883899 | −0.003069577 | | cg06814287 | 0.015932008 |
| | cg00911794 | 0.004601918 | | cg26151597 | 6.54E−05 | | cg05800683 | 0.002444724 |
| | cg03189210 | 0.010347494 | | cg25954627 | −0.002669877 | | cg09135695 | −0.002342218 |
| | cg12962355 | −0.000948809 | | cg04850366 | 0.006363527 | | cg26567592 | −0.000476455 |
| | cg14098681 | 0.004795588 | | cg07516470 | 0.007483227 | | cg09476092 | −0.010555461 |
| | cg19956166 | −0.001562198 | | cg08870743 | 0.011050152 | | cg01070078 | 0.005698293 |
| | cg03128635 | 5.38E−05 | # | cg22647713 | 0.020418454 | | cg18758230 | 0.001052439 |
| | cg05945782 | 0.004137411 | | cg15848031 | 0.004646013 | | cg05500125 | −0.001781246 |
| | cg10753764 | 0.001032054 | | cg16829453 | 0.004383308 | | cg13726504 | −0.01226509 |
| | cg01696193 | 0.001646526 | | cg05775675 | 0.004575359 | | cg17764989 | 0.009218488 |
| | cg17811310 | 0.005650614 | | cg18757695 | 0.008442959 | | cg04897742 | 0.000816033 |
| | cg26036626 | 0.001192759 | | cg06065141 | 0.004129004 | | cg23621097 | 0.003482878 |
| | cg00940313 | 0.006037753 | | cg24113409 | 0.007258795 | | cg04729913 | 0.010516939 |
| | cg04589660 | −0.008444959 | | cg15543281 | 0.001287287 | | cg19759549 | 0.002203064 |
| | cg04988206 | 0.00437912 | | cg20293942 | 0.002111672 | | cg18729886 | 0.001296942 |
| | cg22253838 | 0.006715895 | | cg24311272 | −0.000480991 | | cg25580342 | −0.006501681 |
| | cg18787914 | 0.002089513 | | cg26579986 | 0.012256518 | | cg13822158 | −0.004930104 |
| | cg22783180 | 0.009815768 | | cg11190071 | −0.00516592 | | cg23950233 | 0.011115291 |
| | cg02151609 | 0.006109278 | | cg23039227 | 0.002878957 | | cg23001000 | 0.004910825 |
| | cg15803869 | −0.001350646 | | cg14016875 | 0.005069238 | | cg19962565 | 0.001649262 |
| | cg26784201 | 0.007532044 | | cg07925823 | 0.001060515 | | cg13443605 | 0.004375898 |
| | cg24045369 | 0.002862209 | | cg25755953 | 0.004618932 | | cg19087463 | 0.001320829 |
| | cg19842216 | 0.001141117 | | cg06283368 | 0.003237773 | | cg11772919 | 0.00219493 |
| | cg09535924 | 0.01793183 | | cg10426422 | −0.003868005 | | cg05415308 | 0.001111855 |
| | cg12881557 | 0.001514369 | | cg26709950 | 0.001778895 | | cg19315863 | 0.01325679 |
| | cg13523649 | −0.000685474 | | cg20383624 | 0.002368472 | | cg04263436 | 0.007654523 |
| | cg19305488 | 0.003143098 | | cg17604312 | 0.002352699 | | cg15891218 | 0.004122954 |
| | cg14448169 | −4.05E−05 | | cg09785344 | −0.001648241 | | cg11441553 | 0.004641259 |
| | cg12472603 | −0.004593404 | | cg22560193 | 0.002095656 | | cg02339682 | 0.002088272 |
| | cg12841273 | 0.001414894 | # | cg01724566 | 0.006341603 | | cg08347183 | 0.002328892 |
| | cg06022942 | 0.003440963 | | cg01364137 | −0.011095985 | | cg26977644 | 0.002833119 |
| | cg14294250 | 0.001571045 | | cg13425637 | −0.000964798 | | cg18088653 | 0.003134038 |
| | cg13951527 | 0.001423674 | | cg23943136 | 0.011625265 | | cg14809226 | 0.006563274 |
| | cg01102073 | 0.004168908 | | cg17739038 | 0.001455862 | | cg09376537 | 0.004635002 |
| | cg15140191 | 0.002043608 | | cg05238769 | 0.002277768 | | cg04983516 | 0.00072139 |
| | cg02242344 | 0.003952092 | | cg08711175 | 0.002145074 | | cg22000330 | 0.00888841 |
| | cg07150314 | 0.00043489 | | cg09860653 | 0.000251873 | | cg13484546 | 0.005890146 |
| | cg06659614 | 0.002227856 | | cg17346177 | 0.003507611 | | cg17124583 | 0.015979651 |
| | cg02992881 | 0.001218273 | | cg00040007 | 0.004707271 | | cg25432975 | 0.001132613 |
| | cg24646556 | −0.003936079 | | cg21538208 | 0.004257089 | | cg01504836 | −0.000905563 |
| | cg03010186 | −0.000360435 | | cg03106313 | −0.000177047 | | cg21859603 | 0.011986806 |
| | cg26381352 | 0.001985722 | | cg11468462 | −0.002373353 | | cg02788401 | −0.002122591 |
| | cg16537676 | −0.004758214 | | cg17182507 | 0.009545197 | | cg18086594 | 0.006730021 |
| | cg14891195 | −0.001829673 | | cg15890882 | 0.004592809 | | cg04414274 | 0.005332496 |
| | cg02956248 | 0.00292666 | | cg00052772 | 0.004917845 | # | cg17416730 | 0.009889708 |
| | cg11122493 | −0.000837881 | | cg08509237 | 0.008411432 | | cg20914572 | 0.012306872 |
| | cg19623360 | −0.001196649 | | cg24045832 | 0.000750932 | | cg12568595 | −0.002251153 |
| | cg06994420 | 0.005069164 | | cg17960080 | 0.007326597 | | cg21037008 | 0.007796413 |
| | cg14749448 | 0.000134171 | | cg04778194 | 0.007297137 | | cg16437908 | 0.00160617 |
| | cg17329164 | 0.000244521 | # | cg17603502 | 0.010380725 | | cg20162206 | −0.005310834 |
| | cg19241689 | 0.008494714 | | cg02027735 | −0.002809245 | | cg12847793 | 0.001826159 |
| | cg24039697 | 0.002515546 | | cg00908927 | −0.000155708 | | cg14556146 | 0.002488379 |
| | cg19450714 | 0.003573502 | | cg07028869 | 0.002635514 | | cg02989257 | 0.008471781 |
| | cg10074727 | 0.005998468 | # | cg00403498 | 0.007898345 | | cg26958236 | 0.004684085 |
| | cg11145160 | 0.008355254 | | cg23047693 | 0.004853684 | | cg23074048 | −0.000203767 |
| | cg16993220 | 0.001383258 | | cg17161421 | 0.00800532 | | cg12225685 | −0.000598027 |
| | cg01168201 | 0.001926246 | | cg09965419 | −0.002045705 | | cg19215110 | 0.002002695 |
| | cg17229678 | 0.001473362 | | cg20924286 | −0.000975117 | | cg01461067 | −0.006050793 |
| | cg00702638 | 0.002782649 | | cg13855261 | 0.004898806 | | cg26476820 | 0.004706885 |
| | cg17416280 | −0.001151163 | | cg08483834 | 0.01086478 | | cg13690241 | −0.001044071 |
| | cg10551329 | 0.004477573 | | cg00932104 | −0.004000027 | | cg01312445 | 0.007037249 |
| | cg02901177 | 0.00150142 | | cg08755743 | 0.000649338 | | cg09829319 | 0.002705505 |
| | cg01176516 | 0.000174602 | | cg02919960 | −0.00931745 | | cg04765277 | 0.004785091 |
| | cg11800635 | −0.001056298 | | cg01803928 | 0.005241546 | | cg12064947 | 0.020016096 |
| | cg15690347 | −8.22E−05 | | cg05099909 | −0.001514778 | | cg03244036 | 0.000230501 |
| | cg26270195 | 0.005760773 | | cg01684248 | 0.006500967 | | cg05457563 | 0.004276337 |
| | cg26292521 | 0.008685378 | | cg01389917 | 0.005128332 | | cg12052258 | −0.003680491 |
| | cg09410389 | 0.011307218 | | cg20120165 | 0.001779632 | | cg19882268 | 0.010858446 |
| | cg24303888 | 0.00558661 | | cg11977634 | −0.003829395 | | cg12776287 | 0.003479574 |
| | cg07555797 | 0.008345644 | | cg16004427 | 0.001260938 | | cg03682712 | 0.003297812 |
| | cg19679989 | 0.001006991 | | cg23777946 | 0.003317806 | | cg00881300 | 0.003368168 |
| | cg09042577 | 0.001720342 | | cg20096208 | −0.005310912 | | cg16771406 | 0.00062114 |
| | cg23953820 | −0.003062409 | | cg14914519 | 0.011429763 | | cg08699270 | −0.00266739 |
| | cg10541674 | −0.005498588 | | cg11386011 | 0.004230456 | | cg21518937 | 0.002120422 |
| | cg16553500 | 0.006000178 | | cg25878441 | −0.001099666 | | cg26011438 | −0.001049973 |
| | cg14435807 | 0.001841432 | | cg23359714 | 0.006820596 | | cg07484485 | 0.006814521 |
| | cg02121330 | −0.003283271 | | cg21145624 | −0.006791503 | | cg10094078 | 0.01033447 |
| # | cg16501308 | 0.011701615 | | cg06642647 | 0.008910065 | | cg11444332 | 0.004166015 |
| | cg18124917 | −0.00592583 | | cg22538396 | −0.006447743 | | cg13353999 | 0.008078182 |
| | cg04579211 | 0.007341857 | | cg13882090 | 0.011792496 | | cg17863312 | 0.002622773 |
| | cg23484268 | −0.008299475 | | cg12073479 | −0.00442617 | | cg10982590 | −0.001543983 |
| | cg17991695 | 0.009547913 | | cg00831247 | 0.001020302 | | cg24104433 | 0.000960755 |
| | cg01760756 | −0.002632062 | | cg10948797 | 0.004968048 | | cg22238923 | −0.002377416 |
| | cg06012011 | 0.001608231 | | cg00592510 | 0.003321744 | | cg20981412 | 0.001243186 |
| | cg23117796 | −0.00455367 | | cg06777844 | −0.000770924 | | cg06897686 | 0.01068885 |
| | cg01807770 | 0.006472716 | | cg26912426 | 0.003631565 | | cg06023994 | 0.01813362 |
| | cg20733077 | 0.002727254 | | cg09852607 | 0.006090472 | | cg02115911 | −0.001210189 |
| | cg11453400 | 0.001855053 | | cg01025836 | 0.002211515 | | cg01606023 | 0.005270264 |
| | cg17509807 | 0.007986195 | | cg25608490 | −0.001826877 | | cg19965948 | 0.004548712 |
| | cg21057046 | −0.001523856 | | cg00316759 | 0.008831038 | | cg05784157 | −0.001641587 |
| | cg14040722 | 0.007786271 | | cg22322679 | 0.008905093 | | cg19100596 | 0.000313337 |
| | cg09573795 | 0.006926781 | | cg02551743 | 0.010479475 | | cg11014463 | 0.007986323 |
| | cg10935762 | 0.00496252 | | cg06964816 | −0.003148842 | | cg06685968 | 0.001959438 |
| | cg13329862 | −0.001148365 | | cg11530564 | 0.009405121 | | cg24995976 | 0.003124736 |
| | cg04536704 | 0.010151234 | | cg16836355 | 0.001775388 | | cg22609784 | 0.004397343 |
| | cg26572811 | 0.008052044 | | cg20863107 | 0.00247101 | | cg17967261 | −0.001002268 |
| | cg16403860 | 0.000439126 | | cg12359077 | 0.003732118 | | cg02317742 | −0.004849873 |
| | cg00862597 | −0.001280873 | | cg00927777 | 0.007252409 | | cg27361964 | −6.63E−05 |
| | cg18951187 | −0.001287561 | | cg20196291 | 0.000935989 | | cg02460426 | 0.004773069 |
| | cg13785883 | 0.001002451 | | cg22801992 | 0.001736825 | # | cg06230736 | 0.006620009 |
| | cg13993643 | 0.016204133 | | cg20328456 | 0.00507898 | | cg11001769 | −0.000274747 |
| | cg14023774 | 0.012440082 | | cg13438549 | 0.006424976 | | cg25562834 | 0.003306898 |
| | cg02836487 | 0.005421954 | | cg07841173 | 0.013274197 | | cg12165758 | 0.003083291 |

1. A method for reducing chronic allograft injury in a subject, the method comprising:
determining the DNA methylation level of a CpG panel, comprising at least 4 CpGs from the list shown in Table 4, in a sample from the allograft,
calculating a methylation risk score (MRS) via the sum of methylation values of each CpG in the CpG panel,
comparing the MRS of the sample of the allograft with a reference population of allografts,
determining that the MRS is at least two-fold higher than the lower tertile of the reference population; and
treating the subject with an inhibitor of DNA methylation or hypermethylation, a stimulator of ten-eleven translocation enzyme activity, and/or an inhibitor of Branched-chain aminotransferase 1.

2. The method according to claim 1, wherein the CpG panel, further comprises 29 CpGs as listed in Table 4, or 413 CpGs as listed in Table 3, or 1238 CpGs as listed in Table 6, or 1634 CpGs as listed in Table 2.

3. The method according to claim 1, wherein the allograft is a kidney.

4. The method according to claim 1, wherein the sample from the allograft is taken at the time of implantation in the subject, or is taken post-implantation.

5.-11. (canceled)

12. A kit for determining the DNA methylation level of a CpG panel, the kit comprising probes or primers to measure the CpG methylation level of at least 4 CpGs from the list shown in Table 4.

13-16. (canceled)

17. The method of according to claim 1, wherein the sample from the allograft is a biopsy sample from the allograft.

18. The method of according to claim 1, wherein the sample from the allograft is a liquid biopsy sample from the allograft.

19. The method according to claim 1, wherein the inhibitor of hypermethylation is 5-azacytidine or decitabine.

20. The method according to claim 1, wherein the inhibitor of Branched-chain aminotransferase 1 is ERG240

21. The method according to claim 1, wherein the stimulator of ten-eleven translocation enzyme activity is oxygenation.
